(19) United States
(12) Patent Application Publication
(10) Pub. No.: US 2026/0258066 A1
(43) Pub. Date: Sep. 3, 2026

(54) NOVEL RUTHENIUM COMPLEXES, METHOD OF THEIR SYNTHESIS, INTERMEDIATE COMPOUNDS USED IN THIS METHOD, METHOD OF THEIR SYNTHESIS AND THE USE OF NOVEL RUTHENIUM COMPLEXES IN OLEFIN METATHESIS REACTIONS

(71) Applicant: UNIWERSYTET WARSZAWSKI, Warszawa (PL)

(72) Inventors: Karol GRELA, Warszawa (PL); Anna KAJETANOWICZ, Warszawa (PL); Adrian SYTNICZUK, Minsk Mazowiecki (PL); Mariusz MILEWSKI, Pruszkow (PL); Vishal PUROHIT, Gujarat (IN); Filip STRUZIK, Warszawa (PL)

(21) Appl. No.: 18/876,875

(22) PCT Filed: Jun. 23, 2023
(86) PCT No.: PCT/IB2023/056530

§ 371 Date: Dec. 19, 2024

(30) Foreign Application Priority Data
Jun. 23, 2022 (PL) PL441535

(51) Int. Cl. C07F 15/00; B01J 31/22; C07C 67/333; C07D 207/20

(52) U.S. Cl. CPC C07F 15/0046; B01J 31/2278; C07C 67/333; C07D 207/20; B01J 2231/54; B01J 2531/821; C07C 2601/10

(57) ABSTRACT

The subject of the invention is a novel ruthenium complex with the general formula Ru-l, in which all the variables have the meaning defined in the disclosure. The subject of the invention is also a synthesis method of the ruthenium complex, an intermediate which is a precursor of the ligand used in the synthesis reactions of the ruthenium complex CAAC-l and application of this ruthenium complex as a (pre)catalyst in olefin metathesis reactions.

The subject of the invention are novel ruthenium complexes with CAAC type ligands (Cyclic Alkyl Amino Carbene) which have found a widespread use as catalysts and/or (pre)catalysts of olefin metathesis reactions and their use in olefin metathesis reactions. The subject of the invention also includes intermediate compounds used to synthesise novel ruthenium complexes with CAAC ligands, as well as the method for synthesis of novel ruthenium complexes with CAAC ligands. This invention is used as desired tool in the widely understood organic synthesis, in the selective synthesis of olefins with a C═C bond, in particular in cross metathesis reaction with ethylene-ethenolysis.

Significant progress has been made in recent years in the applications of olefin metathesis in organic synthesis [R. H. Grubbs (Editor), A. G. Wenzel (Editor), D. J. O'Leary (Editor), E Khosravi (Editor), Handbook of Olefin Metathesis, 2. Edition, 3 volumes, 2015, Wiley-VCH Verlag GmbH & Co. KGaA, 1608 pages]. Many ruthenium-based homogeneous olefin metathesis catalysts are known in the state-of-the-art which show high activity in various types of metathesis reactions, as well as high tolerance to functional groups present in the substrate/product. Thanks to the combination of these features, metathesis catalysts are of key importance in modern organic synthesis and in the industry. The most widely present ruthenium complexes in the literature and the ones finding the most widespread use in olefin metathesis reactions include Grubbs-type ruthenium complexes (Gru-I, Gru-II and Gru-III), Hoveyda-Grubbs complexes (Hov-I and Hov-II), as well as indenylidene complexes (Ind-I, Ind-II and Ind-III), 1^{st}, 2^{nd} and 3^{rd} generation [Grubbs et al. Chem. Rev. 2010, 110, 1746-1787; Nolan et al. Chem. Commun. 2014, 50, 10355-10375]. In other cases, most structures of olefin metathesis catalysts are derived from the aforementioned ruthenium complexes. Recently, Bertrand-type catalysts with cyclic (alkyl)(amino)carbenes (CAAC) found an important place in modern organic synthesis thanks to their applications i.e. in cross metathesis reactions and in ring-closing metathesis [Grubbs et at. Chem. Rev., 2010, 110, 1746-1787; WO2017055945A1].

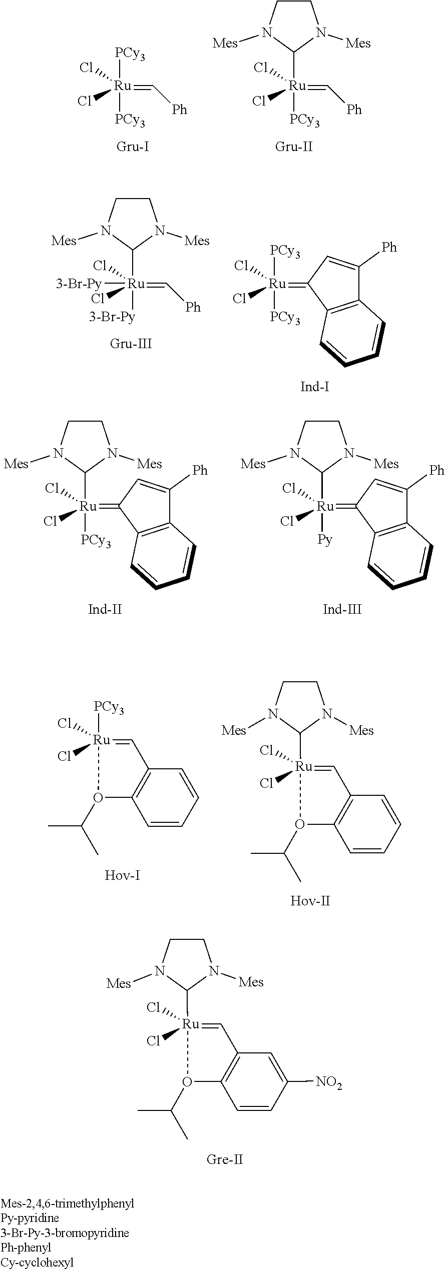

In modern organic synthesis, in the age of ever more shrinking resources, i.e. fossil fuels, and thus of the risk of the lack of the raw materials for synthesis, e.g. polymers based on crude oil derivatives, it is extremely important to develop new technologies and reactions enabling the synthesis of target compounds. Such processes include the ethenolysis reaction, in particular ethenolysis of methyl/ethyl esters of long chain fatty acids. Ruthenium complexes with CAAC ligands in the coordination sphere of ruthenium are particularly useful for the purpose. The first literature report on olefin metathesis ruthenium catalysts with CAAC ligands dates back to 2005 [Bertrandt i in. Angew. Chem. Int. Ed., 2005, 44, 5705-5709]. In this research publication, Bertrand described for the first time CAAC ligands and their use in organic synthesis. In a subsequent publication from 2007 [Bertrandt et al. Angew. Chem. Int. Ed., 2007, 46, 7262-7265] Bertrandt described for the first time a synthesis of ruthenium complexes with CAAC ligands. In both cases, the ligands contained 2,6-diisopropylbenzene at the nitrogen atom, while two methyl (Ru5) substituents and a cyclohexyl substituent (Ru10) at the carbon atom C2. Next, the team of Prof. Bertrandt, in cooperation with the team of Prof. Grubbs, obtained and tested 17 structures in terms of their activity in the ethenolysis reaction of methyl oleate, in which the CAAC ligand contained symmetric substituents at the nitrogen atom: mesityl, 2,6-diisopropylbenzene, 2,6-diethylbenzene, as well as asymmetric ones 2-ethyl-6-methylbenzene, 2-isopropyl-6-methylbenzene, 2-methyl-6-tertbutylbenzene, while at the C2 carbon atom, two of the following substituents: methyl, ethyl, n-propyl, cyclohexyl, adamantyl or phenyl [Bertrandt et al. Angew. Chem. Int. Ed., 2015, 54, 1919-1923]. These catalysts showed high activity towards double C═C bonds in methyl oleate in the presence of positive pressure of ethylene, leading to the formation of valuable industrial products in the olefin metathesis reaction: 1-decene and methyl ester of 9-decenoic acid.

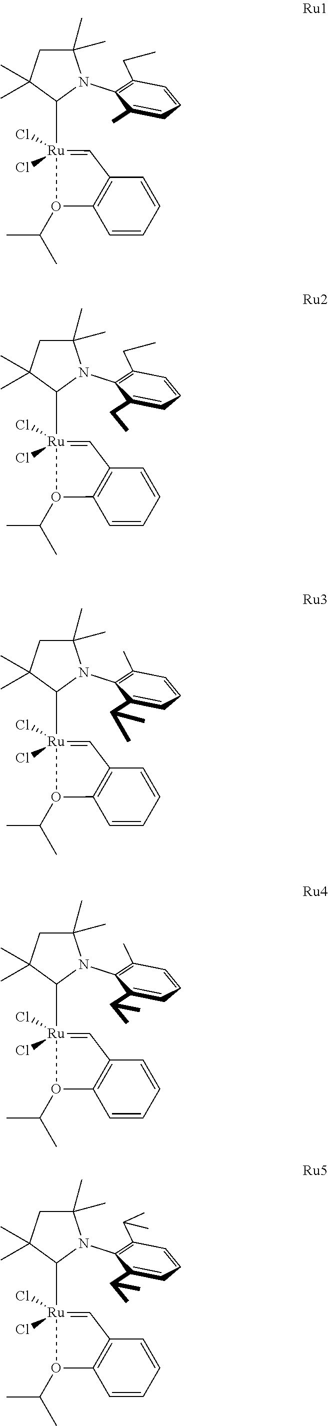

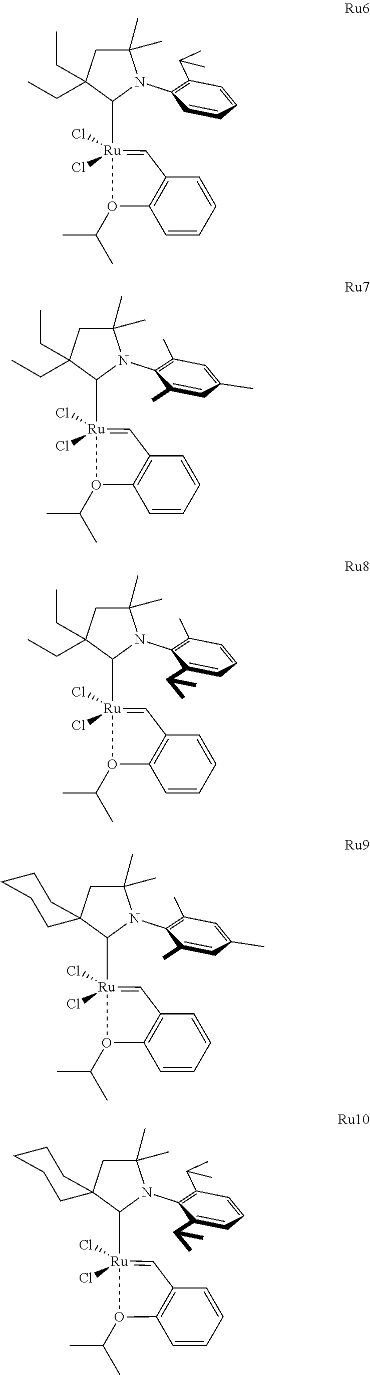

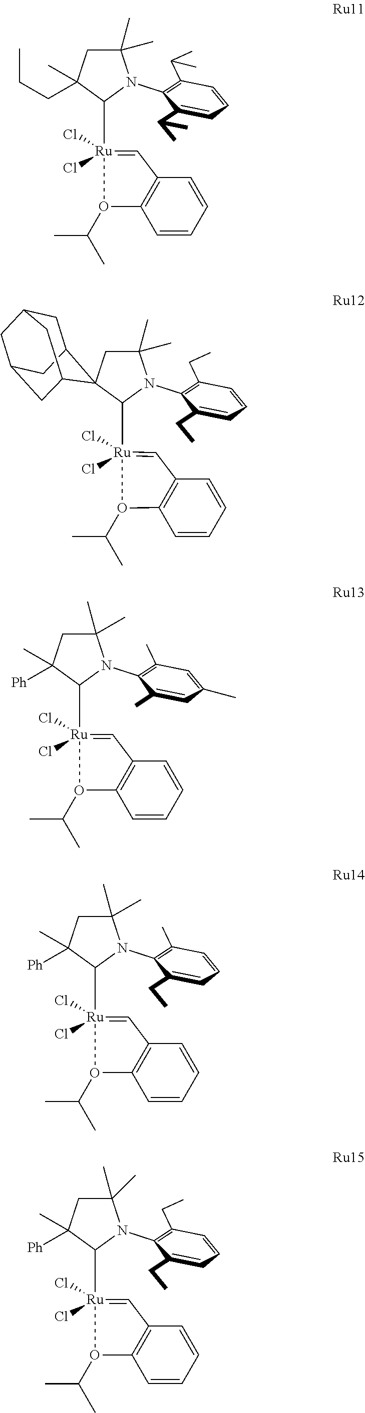

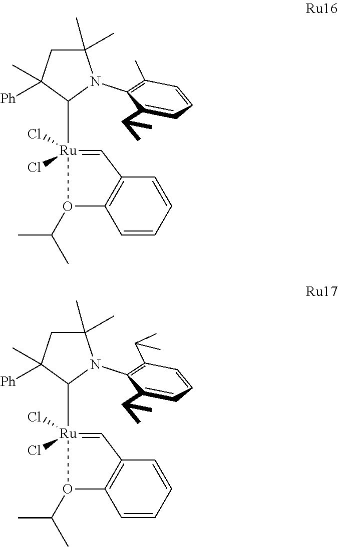

In 2017, Gawin et al. published for the first time a synthesis of indenylidene type complexes with two CAAC ligands [Gawin et al., Angew. Chem. Int. Ed. 2017, 56, 981-986 and EP3356379B1]. This publication presented a synthesis of indenylidene complexes, as well as their activity in selected metathesis reactions, including macrocyclisation, ethenolysis, or cross metathesis reaction of α-olefins. This document also discloses a novel approach to the synthesis of Hoveyda-Grubbs catalysts with CAAC ligands, involving thermal dissociation of one CAAC ligand in the indenylidene complex, followed by reaction of the intermediate with the relevant styrene.

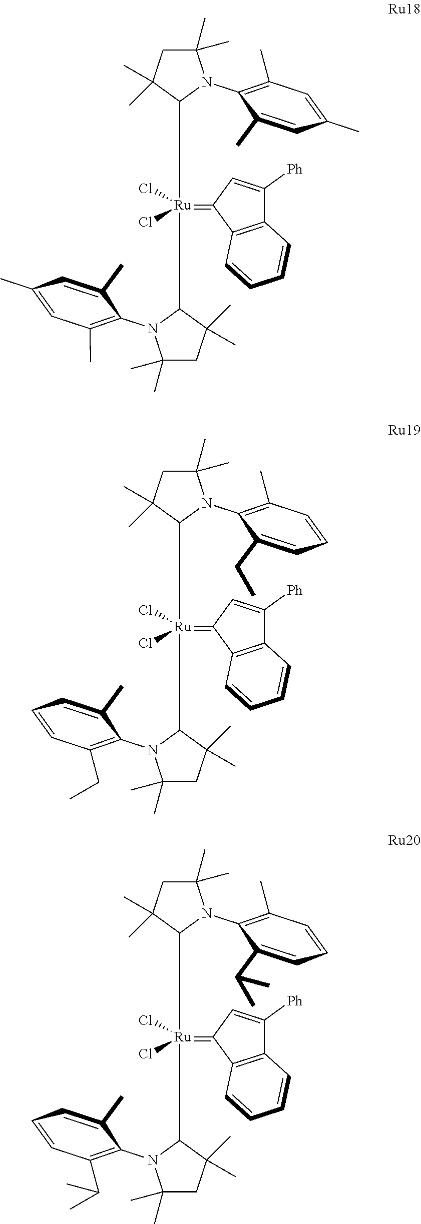

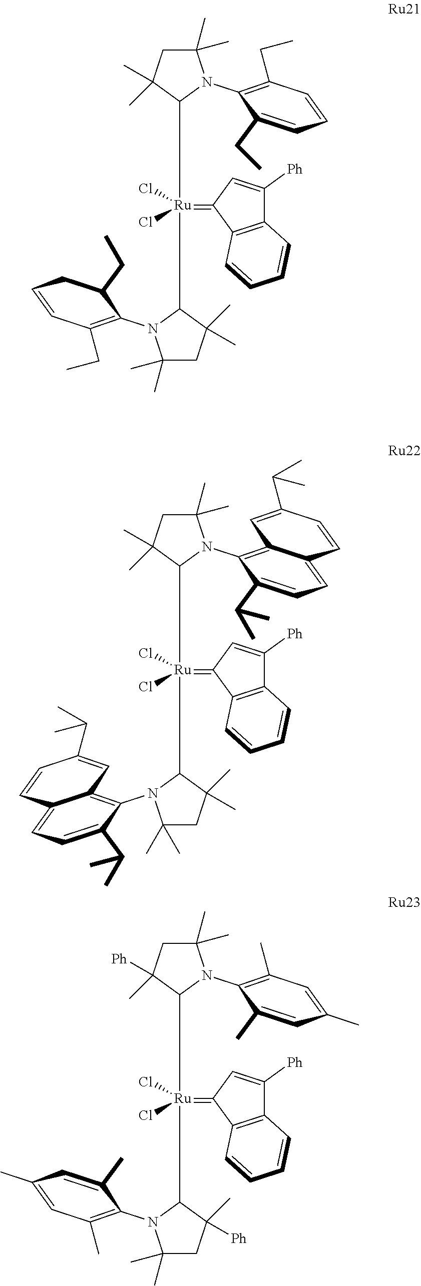

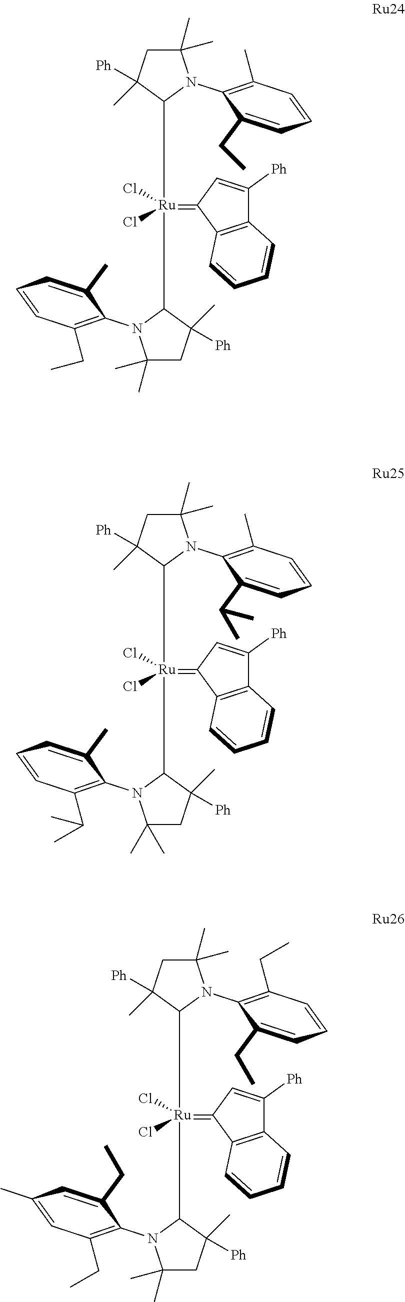

Also in 2017, Gawin et al. published a synthesis method for Hoveyda-Grubbs type complex analogues with a nitro group in the para position as substrates using BisCAAC complexes [Gawin et al. ACS Catal. 2017, 7, 5443-5449]. These complexes proved to be effective in macrocyclisations and cross metathesis reactions with acrylonitrile.

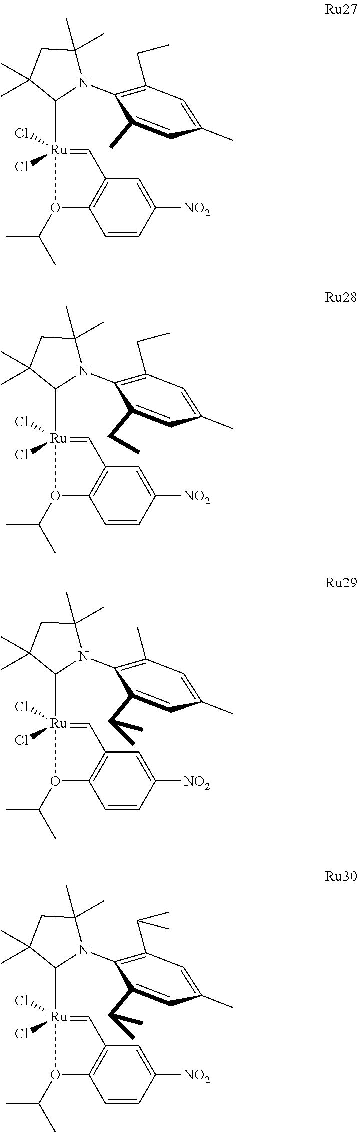

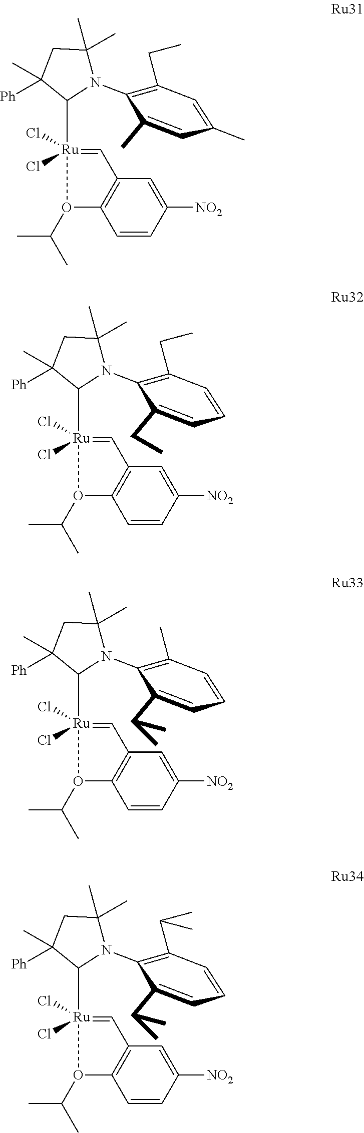

The European patent [EP3356379B1] discloses the structures Ru35-Ru37 with a modified benzylidene fragment.

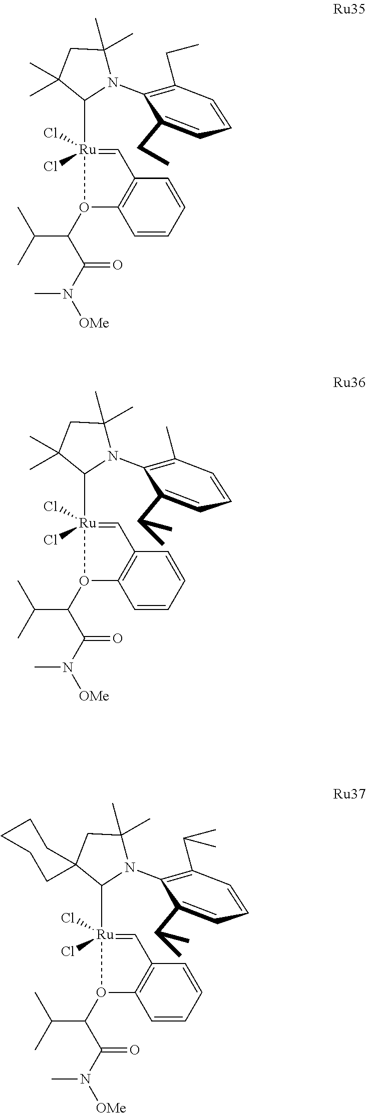

Subsequent modifications of ruthenium catalysts were related to the Hoveyda-Grubbs type complexes, wherein the hydrogen atom in the styrene part was replaced with an EWG or an EDG group. By modulating the nature of the styrenyl ether ligand, Mignagni et al. studied the reactivity of Ru38 and Ru39 catalysts [FR2947189A1; FR2934178A1]. It was shown that the presence of amine group donating electrons has a negative impact on the catalytic activity. On the other hand, the modification of Ru40 with an electron acceptor SO₂NMe₂ group (Zhan type catalyst) enabled the synthesis of an extremely active catalyst in the ethenolysis reaction of fatty acids [EP1905777B1; US2011/0306815A1].

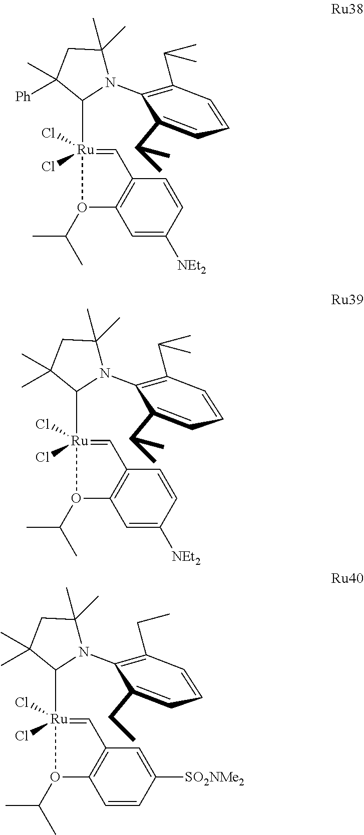

Verpoort et al. studied the impact of labile chelating groups: benzyl ether, benzyl thioether, and benzylamine [WO2017/185324A1]. All catalysis transformed methyl oleate with high selectivity and high TON values [Turnover number—the number of catalytic cycles, the number of moles of substrate undergoing reactions, calculated per one mole of the catalyst](between 180,000 and 210,000). A reaction performed with ethylene (99.995%), in the presence of Ru43 chelated with benzylamine and an activator (HSiC₃), yielded the highest recorded thus far TON value (390,000).

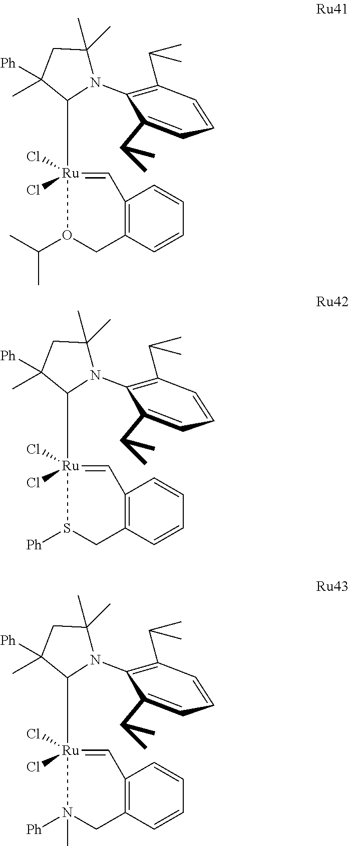

Lemcoff et al. showed that analogues of Hoveyda-Grubbs type catalysts chelated with sulphur in the benzylidene part are present in the cis/trans pairs Ru44-Ru47. The activity of complexes was studied i.e. in polymerisation reactions of norbornene derivatives [Rozenberg, I. et al. ACS Catal. 2018, 8, 8182-8191.].

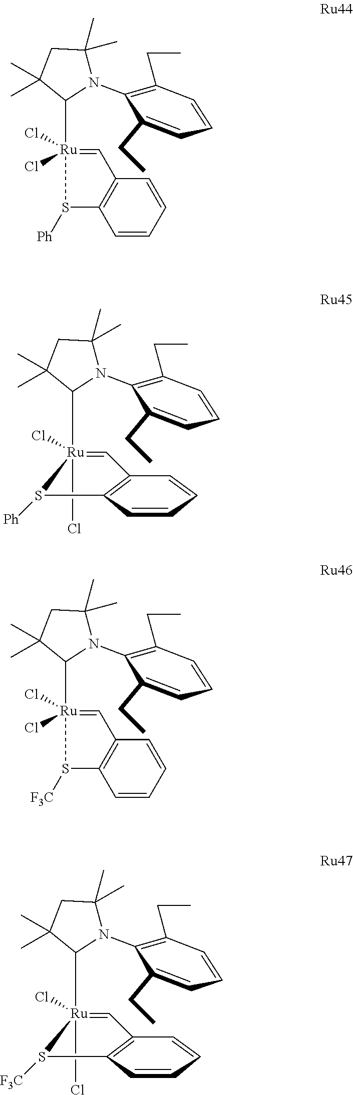

Tuba et al. developed a tandem ethenolysis/isomerisation reaction using catalysts based on bicyclic (alkyl)(amino)carbenes (BICAAC) (Ru48-Ru52) and the (RuHCl(CO)—(PPh₃)₃ catalyst. Thanks to the used methodology and using methyl oleate as the substrate, they were able to obtain methyl acrylate and propylene as the main products, achieving TON value of 1400 (Nagyhazi et al. Angew Chem. Int. Ed. 2022, 61, e202204413).

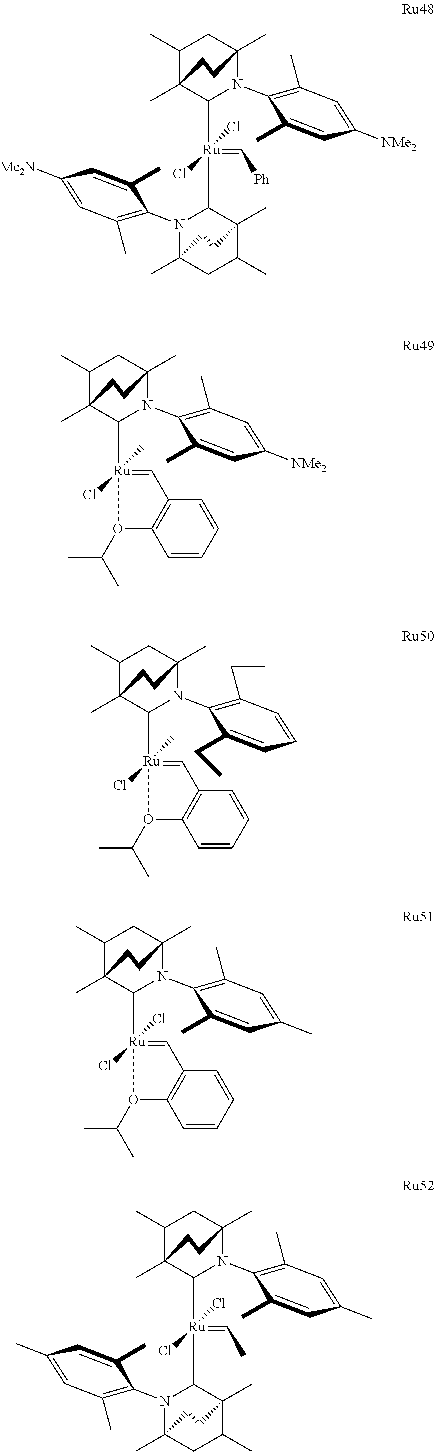

The limited availability of a wide range of anilines used as substrates in the synthesis of CAAC ligands is a significant problem known in the art. Their use is largely limited to simple and symmetric anilines, i.e. 2,4,6-trimethylaniline, 2,6-diethylaniline or 2,6-diisopropylaniline. The high prices and low availability of asymmetric aniline derivatives, in particular of anilines containing branched alkyl substituents, e.g. containing branched alkyl substituents, e.g. 2-isopropyl-6-methylaniline or 2-isopropyl-6-ethylaniline are a problem in the design and synthesis of novel CAAC ligands. Another problem includes the long and complicated synthesis paths for these and other aniline derivatives, in which the expected products are obtained with low yields, significantly limiting the design options related to novel catalysts. These characteristics pose a significant limitation to the further development of metalloorganic catalysis based on ruthenium complexes with novel CAAC ligands. Obtaining novel catalysts for olefin metathesis with ligands based, in particular, on asymmetric anilines with planned properties becomes difficult to implement in chemical synthesis and economically unjustified in industrial scaling.

In the search for novel ruthenium complexes with high catalytic activity and improved durability and selectivity enabling high TON values to be obtained, it is important that convenient synthetic paths based on easily available and inexpensive substrates lead to these compounds. From the point of view of industrial scaling, it is also important for the planned synthesis to be efficient at every stage and that the reaction products can be purified in a simple way, using techniques such as crystallisation or distillation. It is also important to expand the library of ligands, the application of which will comprise an alternative and/or improved source of structures of ruthenium complexes used as catalysts in i.e. ethenolysis reactions of ester derivatives of fatty acids.

The low solubility of known ruthenium complexes in non-polar environment of the reaction (e.g. paraffins, n-hexane, cyclohexane, n-heptane, n-decane, etc. or in many olein metathesis substrates) is an equally important problem known in the art. Homogeneous metathesis reaction are usually conducted in a polar media, in chlorinated solvents (dichloromethane, 1,2-dichloroethane) or aromatic solvents (benzene, toluene), but these solvents are harmful for the environment an hazardous to the users [Green Chem., 2014, 16, 1125-1130]. Thus, alternative catalytical systems are looked for, which may be used in solvents, the use of which conforms to the rules of “green chemistry” and the rules of sustainable growth of the modern, large scale chemical industry [R. A. Sheldon, Green Chem. 2017, 19, 18-43.]. It should also be noted that the solvents routinely used in olefin metathesis processes (dichloromethane, toluene and benzene) are not accepted by the pharmaceutical industry [Eur. J. Org. Chem. 2019, 640-646], where elimination of harmful solvents (classified into ICH classes 1 and 2) is required at every stage of the synthesis of active pharmaceutical ingredients (API).

Additionally, the solubility of known catalysts in some non-polar monomers, such as dicyclopentadiene (DCPD) and tricyclopentadiene (TCPD) or in n-olefins (including those obtained in pyrolysis of polyethylene and polypropylene waste) is usually low, and it is important that ruthenium complexes form homogeneous systems with them.

In the art, there is a clear technical issue related to the poor solubility of ruthenium complexes in non-polar solvents, which leads to the limited ability to use the olefin metathesis process in non-polar environment (i.e. paraffins, plant oils, and others).

It was surprisingly discovered that aniline derivatives with alkyl, branched substituents can be easily obtained in a simple and efficient, three-stage synthesis, the key stage of which is the synthesis of branched aniline derivatives in aza-Claisen reaction. The aniline derivatives obtained via this synthesis and other, similar organic compounds can act as substrates for novel CAAC ligands used in the synthesis of ruthenium catalysts and/or (pre)catalysts of olefin metathesis.

Thus, the subject of this invention is a precursor of cyclic, alkylamine carbenes (CAAC) with the formula CAAC-1

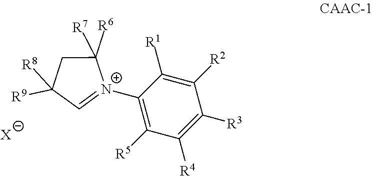

in which
X denotes an anion selected from a group including a halogen anion, BF₄⁻, PF₆⁻, ClO₄⁻, CF₃SO₂O⁻;
R¹, R², R³, R⁴ and R⁵ denote independently a hydrogen atom, a C₁-C₁₂ alkyl group, a C₃-C₁₂ cycloalkyl group, a C₅-C₂₀ aryl group or a C₅-C₂₀ heteroaryl group, a C₅-C₂₅ aralkyl group, which may be independently substituted by one and/or more substituents selected from a group including a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group, a sulphide group (—SR″), an amine group (—NR″₂), in which the R″ group independently denotes a hydrogen atom, C₁-C₅ alkyl, C₆-C₂₄ aryl, C₇-C₂₄ aralkyl, alternatively R¹, R², R³, R⁴ and R⁵ are connected and form a C₅-C₂₅ ring wherein at least one substituent R¹, R² and R⁵ has an alkyl substituent with at least one secondary, tertiary or quaternary carbon atom, preferably at least one substituent R¹, R² and R⁵ has an alkyl substituent with at least two secondary, tertiary or quaternary carbon atoms, more preferably at least one substituent R¹, R² and R⁵ has an alkyl substituent with at least three secondary, tertiary or quaternary carbon atoms, most preferably at least one substituent R¹, R² and R⁵ has an alkyl substituent with at least four secondary, tertiary or quaternary carbon atoms,
each substituent R⁶, R⁷, R⁸ and R⁹ denotes a hydrogen atom, a halogen atom, an alkyl C₁-C₁₂ group or a C₅-C₂₀ aryl group, which may be substituted independently by one and/or more substituents selected from a group including hydrogen, a C₁-C₁₂ alkyl group, a C₁C2 perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently means a hydrogen atom, a C₁-C₅ alkyl, a C₆-C₂₄ aryl, a C₇-C₂₄ aralkyl, alternatively, R¹ and R⁷ and/or R⁸ and R⁹ are connected, forming a C₅-C₂₅ ring group.

Preferably, the precursor of cyclic alkylamine carbenes (CAAC) is represented by the formula CAAC-2 or CAAC-3 or CAAC-4

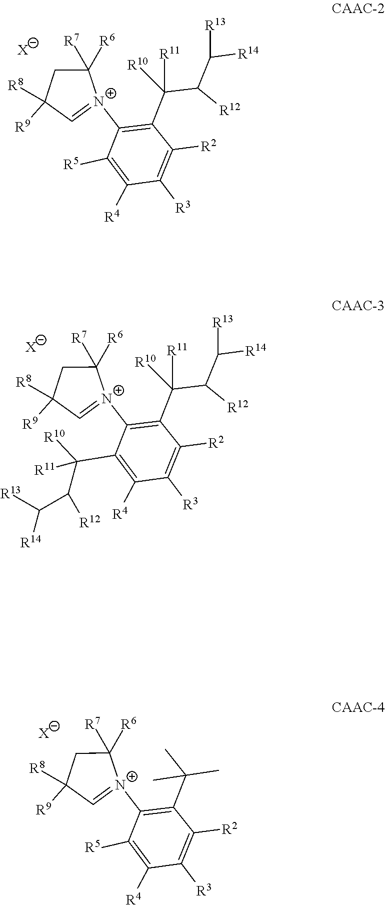

in which the substituents R², R³, R⁴, R⁵, R⁶, R⁷, R⁸ and R⁹ have the meanings defined above,
the R¹⁰, R¹¹, R¹², R¹³ and R¹⁴ substituents denote independently a hydrogen atom, a C₁-C₁₂ alkyl group, a C₃-C₁₂ cycloalkyl group, a C₅-C₂₀ aryl group or a C₅-C₂₀ heteroaryl group, a C₅-C₂₅ aralkyl group, which may be independently substituted by one and/or more substituents selected from a group including a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group, a sulphide group (—SR″), an amine group (—NR″₂), in which the R″ group independently denotes a hydrogen atom, C₁-C₅ alkyl, C₆-C₂₄ aryl, C₇-C₂₄ aralkyl, alternatively R¹, R², R³, R¹³ and R⁵ are connected and form a C₅-C₂₅ ring.

Preferably, the precursor of cyclic alkylamine carbenes (CAAC) is represented by the formula CAAC-5 or CAAC-6 or CAAC-7

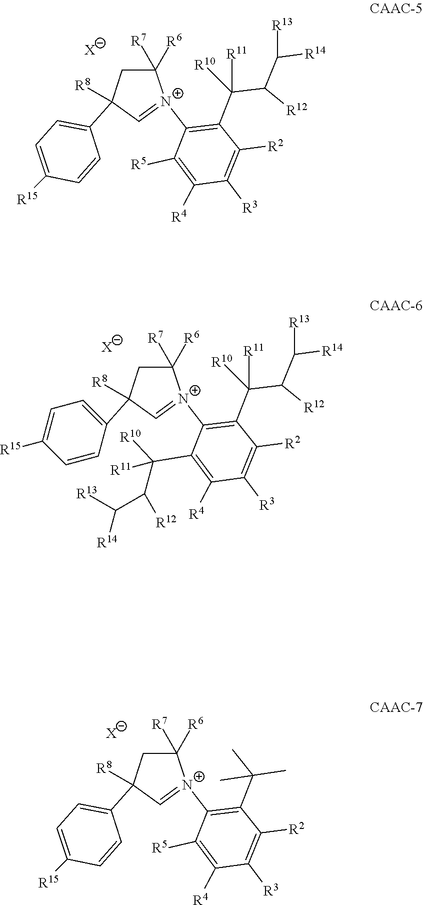

where X denote an anion selected from a group including a halogen atom, BF₄⁻, PF₆⁻, ClO₄⁻, CF₃SO₂O⁻;
and the R², R³, R⁴, R⁵, R⁶, R⁷, R⁸, R⁹, R¹⁰, R¹¹, R¹², R¹³ and R¹⁴ substituents have the meanings defined above,
R¹⁵ independently denotes a hydrogen atom, a C₁-C₁₂ alkyl group, a C₃-C₁₂ cycloalkyl group, a C₅-C₂₀ aryl or a C₅-C₂₀ heteroaryl group, a C₅-C₂₅ aralkyl group, which may be independently substituted by one and/or more substituent selected from a group including a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group, an alkoxy group (—OR″), a sulphide group (—SR″), an amine group (—NR″₂), in which the R″ group independently denotes a hydrogen atom, a C₁-C₅ alkyl, a C₆-C₂₄ aryl, a C₇-C₂₄ aralkyl.

Preferably, the precursor of cyclic alkylamine carbenes (CAAC) is represented by the formula CAAC-a, CAAC-b, CAAC-c, CAAC-d, CAAC-e, CAAC-f, CAAC-g, CAAC-h, CAAC-i, CAAC-j, CAAC-k or CAAC-l:

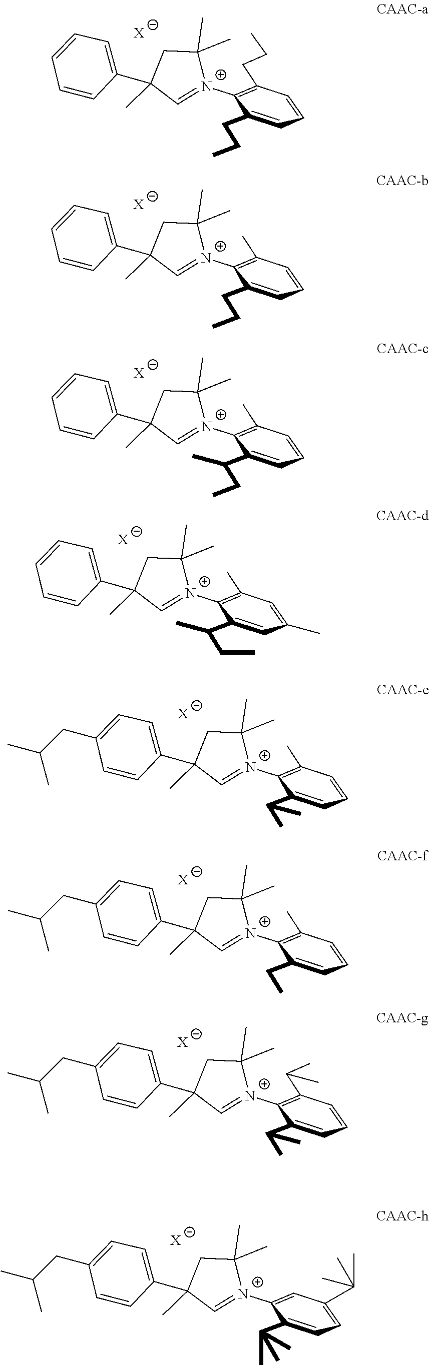

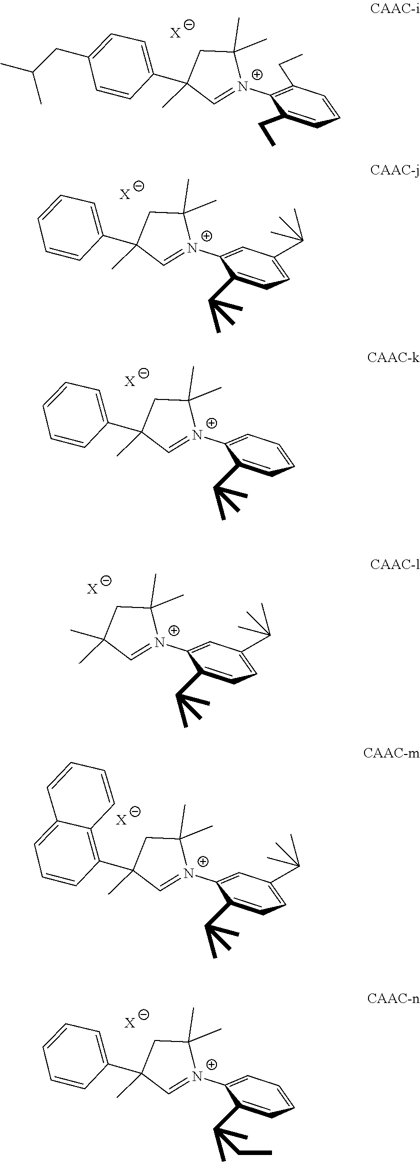

The subject of the invention also includes a synthesis method of a cyclic precursor of alkylamine carbenes (CAAC) with the formula CAAC-2 or CAAC-3 defined above

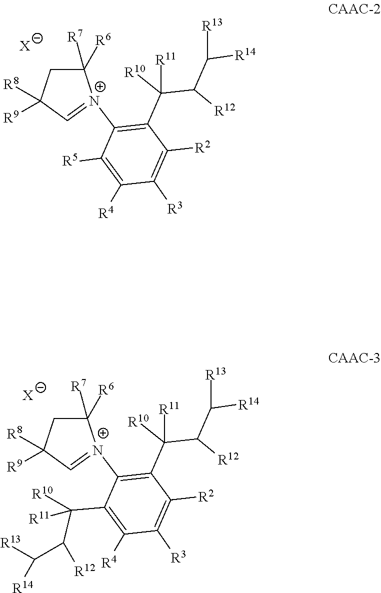

in which
during the first stage, a compound with the formula 2a or 3a

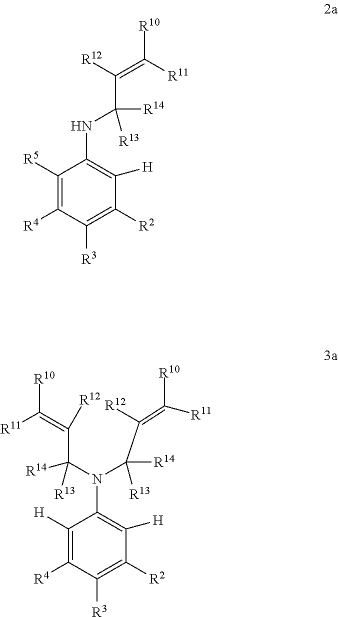

in which the substituents R², R³, R⁴, R⁵, R¹⁰, R¹¹, R¹², R¹³ and R¹⁴ have the meanings defined above are subjected to a thermal aza-Claisen rearrangement in the presence of a Lewis acid, obtaining a compound with the formula 2b or 3b,

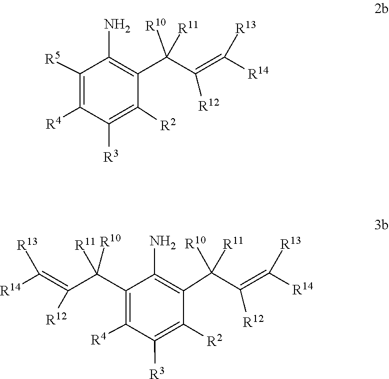

in which the substituents R², R³, R⁴, R¹⁰, R¹¹, R¹², R¹³ and R¹⁴ have the meanings defined above,
which is subsequently subjected in the second reaction stage-reduction of the double bond, using a transition metal catalyst deposited on activated charcoal in the presence of gaseous hydrogen, with the formation of a compound with the formula 2c or 3c

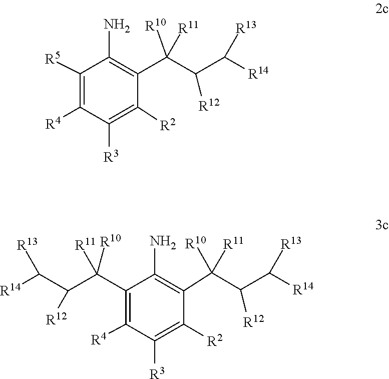

in which the substituents R², R³, R⁴, R¹⁰, R¹¹, R¹², R¹³ and R¹⁴ have the meanings defined above,
which is subjected to a reaction with a compound with formula 4 in the third stage

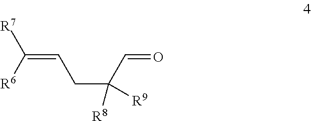

in which R⁶, R⁷, R⁸ and R⁹ have the meanings defined above,
in the presence of a Brönsted acid, with the formation of a compound with the formula 2d or 3d,

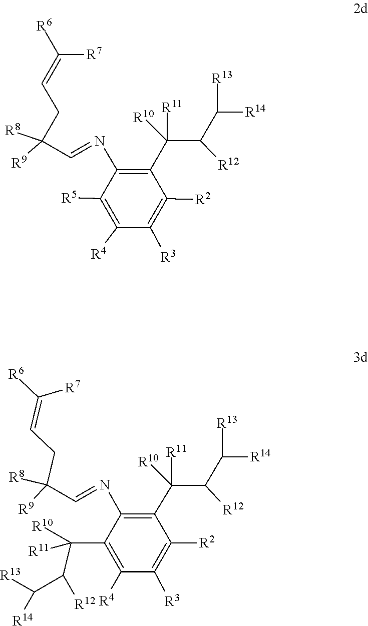

where the R², R³, R⁴, R⁵, R⁶, R⁷, R⁸, R⁹, R¹⁰, R¹¹, R¹², R¹³ and R¹⁴ substituents have the meanings defined above,
which is subsequently subjected to a reaction with hydrochloric acid at an elevated temperature, followed by an anion exchange reaction with an anion selected from a group including a halogen anion, BF₄⁻, PF₆⁻, ClO₄⁻, CF₃SO₂O⁻, with the formation of the relevant precursors of cyclic alkylamine carbenes (CAAC) with the formula CAAC-2 or CAAC-3.

Preferably, a Lewis acid selected from BF₃, B(OR)₃, AlCl₃, MgCl₂, TiCl₄, Ti(OR)₄ is used in the first step; in the second step, the reduction reaction is carried out in the presence of a catalyst selected from Pd, Pt, Rh, Ru, Ag, Au; and in the third step, the reaction is carried out in the presence of a Bronsted acid selected from the para-toluenesulphonic acid, sulphuric acid, hydrochloric acid, trifluoroacetic acid, trifluoromethanesulphonic acid, fluorosulphuric acid.

The subject of the invention also includes a ruthenium complex with the formula 1-Ru

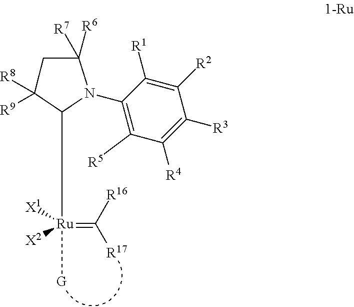

in which:
X¹ and X² independently from each other denote an anionic ligand selected from a group including a halogen anion, a —CN, —SCN, —OR^{a}, —SR^{a}, —O(C=O)R^{a}, —O(SO₂)R^{a} and —OSi(R^{a})₃ group, in which R^{a} denotes a C₁-C₁₂ alkyl, a C₃-C₁₂ cycloalkyl, a C2-C₁₂ alkenyl or a C₅-C₂₀ aryl, which is optionally substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom;
R¹, R², R³, R⁴ and R⁵ denote independently a hydrogen atom, a C₁-C₁₂ alkyl group, a C₃-C₂ cycloalkyl group, a C₅-C₂₀ aryl group or a C₅-C₂₀ heteroaryl group, a C₅-C₂₅ aralkyl group, which may be independently substituted by one and/or more substituents selected from a group including a hydrogen atom, a halogen atom, a C₁-C₁₂ alkyl group, a C₁-C₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group, a sulphide group (—SR″), an amine group (—NR″₂), in which the R″ group independently denotes a hydrogen atom, C₁-C₅ alkyl, C₆-C₂₄ aryl, C₇-C₂₄ aralkyl, alternatively R¹, R², R³, R⁴ and R⁵ are connected and form a C₅-C₂₅ ring
wherein at least one substituent R¹, R² and R⁵ has an alkyl substituent with at least one secondary, tertiary or quaternary carbon atom, preferably at least one substituent R¹, R² and R⁵ has an alkyl substituent with at least two secondary, tertiary or quaternary carbon atoms, more preferably at least one substituent R¹, R² and R⁵ has an alkyl substituent with at least three secondary, tertiary or quaternary carbon atoms, most preferably at least one substituent R¹, R² and R⁵ has an alkyl substituent with at least four secondary, tertiary or quaternary carbon atoms,
each substituent R⁶, R⁷, R⁸ and R⁹ denotes a hydrogen atom, a halogen atom, an alkyl C₁-C₁₂ group or a C₅-C₂₀ aryl group, which may be substituted independently by one and/or more substituents selected from a group including hydrogen, a C₁-C₁₂ alkyl group, a C₁-C₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently means a hydrogen atom, a C₁-C₅ alkyl, a C₆-C₂₄ aryl, a C₇-C₂₄ aralkyl, alternatively, R⁶ and R⁷ and/or R⁸ and R⁹ are connected, forming a C₅-C₂₅ ring group.
R¹⁶ and R¹⁷ independently denote a hydrogen atom, a halogen atom, a C₁-C₂₅ alkyl, possibly substituted, a C₃-C₂₅ cycloalkyl, possibly substituted, a C₁-C₁₂ perfluoroalkyl, possibly substituted, a C₂-C₂₅ alkene, possibly substituted, a C₂-C₂₅ alkenyl, possibly substituted, a C₃-C₂₅ cycloalkenyl, possibly substituted, a C₂-C₂₅ alkynyl, possibly substituted, a C₃-C₂₅ cycloalkynyl, possibly substituted, a C₁-C₂₅ alkoxyl, possibly substituted, a C₅-C₂₅ aryl, possibly substituted, a C₅-C₂₅ aryloxyl, possibly substituted, a C₆-C₂₅ arylalkyl, possibly substituted, a C₅-C₂₅ heteroaryl, possibly substituted, a C₅-C₂₅ heteroaryloxyl, possibly substituted, a C₅-C₂₅ perfluoroaryl, possibly substituted, a 3-12-membered heterocycle containing a sulphur, oxygen, nitrogen, selenium or phosphorus atom, possibly substituted;
wherein the R¹⁶ and R¹⁷ substituents may be connected, forming a ring selected from a group including a C₃-C₂₅ cycloalkyl, a C₃-C₂₅ cycloalkenyl, a C₃-C₂₅ cycloalkynyl, a C₅-C₂₅ aryl, a C₅-C₂₅ heteroaryl, a C₅-C₂₅ perfluoroaryl, a 3-12-membered heterocycle containing a sulphur, oxygen, nitrogen, selenium or phosphorus atom, which may be independently substituted with one and/or more substituents selected from a group including a hydrogen atom, a halogen atom, a C₁-C₂₅ alkyl, a C₃-C₂₅ cycloalkyl, a C₁-C₁₂ perfluoroalkyl, a C₂-C₂₅ alkene, a C₂-C₂₅ alkenyl, a C₃-C₂₅ cycloalkenyl, a C₂-C₂₅ alkynyl, a C₃-C₂₅ cycloalkynyl, a C₁-C₂₅ alkoxyl, a C₅-C₂₅ aryl, a C₅-C₂₅ aryloxyl, a C₆-C₂₅ arylalkyl, a C₅-C₂₅ heteroaryl, a C₅-C₂₅ heteoaryloxyl, a C₅-C₂₅ perfluoroaryl, a 3-12-membered heterocycle;
where the substituents R¹⁶ and R¹⁷ independently and preferably denote a hydrogen atom and/or a C₅-C₂₅ aryl independently substituted with a hydrogen atom, a halogen atom, a C₁-C₂₅ alkyl group, a C₂-C₂₅ alkenyl group, an alkoxy group (—OR″), a sulphide group (—SR″), a sulphoxide group (—S(O)R″), a sulphonium group (—S⁺R″₂), a sulphone group (—SO₂R″), a sulphonamide group (—SO₂NR″₂), an amine group (—NR″₂), an ammonium group (—N⁺R″₃), a nitro group (—NO₂), a cyanide group (—CN), a phosphonate group (—P(O)(OR″)₂), a phoshphinate group (—P(O)R″(OR″)), a phosphonine group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group independently denotes a hydrogen atom, a C₁-C₅ alkyl, a C₁-C₅ perfluoroalkyl, a C₆-C₂₄ aryl, a C₇-C₂₄ aralkyl, a C₅-C₂₄ perfluoroaryl, two R″ groups may be connected, forming a C₃-C₁₂ cycloalkyl or a C₃-C₂₅ heterocycloalkyl ring containing a nitrogen, an oxygen or a sulphur atom, possibly additionally substituted with a C₁-C₁₂ alkyl group, alternatively R″ denote a ketone group (—COR^{c}), in which R^{c} denotes a C₁-C₁₂ perfluoroalkyl or an alkoxy group (—OR^{d}), in which R^{d} denote a C₁-C₁₂ alkyl or a C₃-C₁₂ heterocycloalkyl containing a nitrogen atom, an oxygen atom or a sulphur atom, possibly additionally substituted with a C₁-C₁₂ alkyl group;
G is selected from entities such as
   ligand with the formula CAAC-5 or CAAC-6 or CAAC-7

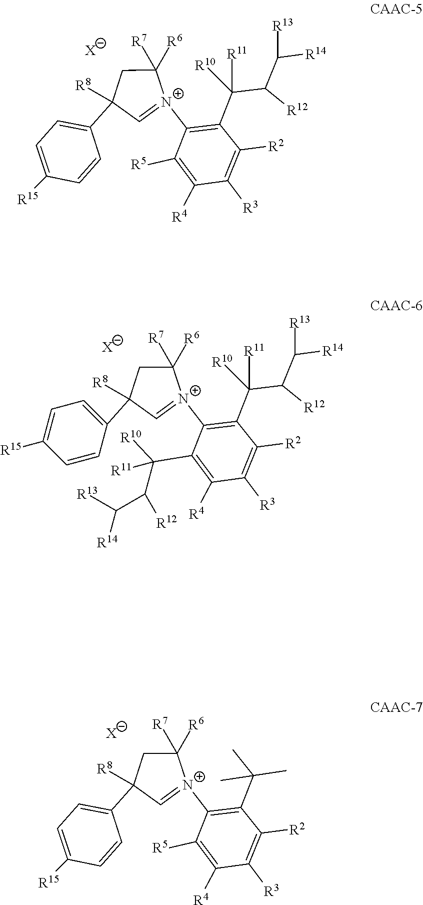

in which X and the substituents R¹ to R¹⁵ have the meanings defined above
or
   heteroatom 1
selected from a group including an oxygen, sulphur, selenium atom, substituted with a group selected from such as a hydrogen atom, a halogen atom, an oxygen atom, a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇—C₂₀ aralkyl, a C₅-C₂₄ aryloxyl, a C₂-C₁₂ alkenyl, a C₆-C₂₀ heteroaryl or a C₅-C₂₄ heteroaryloxyl, a 3-12-membered heterocycle, optionally substituted with an acyl (—COR′), a cyano (—CN), a carboxy (—COOH), an ester (—COOR′), an ester (—CH₂COOR′), an ester (—CHR′COOR′), an ester (—C(R′)₂COOR′), an amide (—CONR′₂), a Weinreb type amide (—CON(R′)(OR′)), a sulphone (—SO₂R′), a formyl (—COH), a sulphonamide (—SO₂NR′₂), a ketone (—COR′), a thioamide (—CSNR′₂), a thioketone (—CSR′), a thionoester (—CSOR′), a thioester (—COSR′), a dithioester (—CS₂R′) group, in which the R′ group denotes independently a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl, a C₅-C₂₄ heteroaryloxyl and then the dashed line denotes a direct bond between the heteroatom and the R¹⁷ substituent or a bond between the R¹⁷ substituent and the heteroatom via methylene bridge —CH₂—, —CHR′— or —CR′₂—, wherein the R¹⁴ substituent is a C₅-C₁₅ aryl, possibly substituted with 1-4 substituents independently selected from a group including a hydrogen atom, a halogen atom, a C₁-C₂₅ alkyl, a C₃-C₂₅ cycloalkyl, a C₂-C₂₅ alkenyl, a C₃-C₂₅ cycloalkenyl, a C₂-C₂₅ alkynyl, a C₃-C₂₅ cycloalkynyl, a C₁-C₂₅ perfluoroalkyl, a C₅-C₂₀ alkoxyl, a C₅-C₂₀ aryl, a C₅-C₂₀ perfluoroaryl, a C7-C₂₀ aralkyl, a C₅-C₂₄ aryloxyl, a C₆-C₂₀ heteroaryl or C₅-C₂₄ heteroaryloxyl, 3-12-membered heterocycle, an alkoxy group (—OR″), a sulphide group (—SR″), a sulphoxide group (—S(O)R″), a sulphonium group (—S⁺R″₂), a sulphone group (—SO₂R″), a sulphonamide group (—SO₂NR″₂), an amine group (—NR″₂), an ammonium group (—N⁺R″3), a nitro group (—NO₂), a cyanide group (—CN), a phosphonate group (—P(O)(OR″)₂), a phosphinate group (—P(O)R″(OR″)), a phosphonine group (—P(OR″)₂), a phosphine group (—PR″₂), a phosphine oxide group (—P(O)R″₂), a phosphonium group (—P⁺R″3), a carboxy group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes a C₁-C₅ alkyl, a C₁-C₅ perfluoroalkyl, a C₆-C₂₄ aryl, a C₇-C₂₄ aralkyl, a C₅-C₂₄ perfluoroaryl; or
   heteroatom 2
selected from a group including a nitrogen atom or a phosphorus atom, substituted with a group selected from groups such as hydrogen atom, methylidene, possibly substituted with a R′ substituent, a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ alkenyl or a C₅-C₂₄ heteroaryloxyl, 3-12-membered heterocycle, an acyl group (—COR′), an ester group (—COOR′), tert-butylcarboxycarbon group (t-Boc) or a 9-fluorenylmethoxycarbonyl group (Fmoc), a carbamine group (—CONR′₂), a sulphone group (—SO₂R′), a formyl group (—COH), in which the R′ group denotes a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl or a C₅-C₂₄ heteroaryloxyl, optionally substituted with an acyl group (—COR′), a cyano group (—CN), a carboxy group (—COOH), an ester group (—COOR′), an ester group (—CH₂COOR′), an ester group (—CHR′COOR′), an ester group (—C(R′)₂COOR′), an amide group (—CONR′₂), a sulphone group (—SO₂R′), a formyl group (—COH), a sulphonamide group (—SO₂NR′₂), a ketone group (—COR′), a thioamide group (—CSNR′₂), a thioketone group (—CSR′), a thionoester group (—CSOR′), a thioester group (—COSR′), a dithioester group (—CS₂R′), in which the R′ group denotes a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl or a C₅-C₂₄ heteroaryloxyl, and then the dashed line denotes a direct bond between a heteroatom and the R¹⁴ substituent or denotes a bond between the R¹⁷ substituent and the heteroatom via a methylene bridge (CH₂)—, —(CHR′)— or (CR′₂)—; wherein the R¹⁷ is an C₅-C₁₂ aryl, possibly substituted with 1-4 substituents independently selected from a group including a hydrogen atom, a halogen atom, a C₁-C₂₅ alkyl, a C₃-C₂₅ cycloalkyl, a C₂-C₂₅ alkenyl, a C₃-C₂₅ cycloalkenyl, a C₂-C₂₅ alkynyl, a C₃-C₂₅ cycloalkynyl, a C₁-C₂₅ perfluoroalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₆-C₂₀ heteroaryl or a C₅-C₂₄ heteroaryloxyl, 3-12-membered heterocycle, an alkoxy group (—OR″), a sulphide group (—SR″), a sulphoxide group (—S(O)R″), a sulphonium group (—S⁺R″₂), a sulphone group (—SO₂R″), a sulphonamide group (—SO₂NR″₂), an amine group (—NR″₂), an ammonium group (—N⁺R″3), a nitro group (—NO₂), a cyano group (—CN), a phosphinous group (—P(O)(OR″)₂), a phosphinic group (—P(O)R″(OR″)), a phosphonine group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes a C₁-C₅ alkyl, a C₁-C₅ perfluoroalkyl, a C₆-C₂₄ aryl, a C₇-C₂₄ aralkyl, a C₅-C₂₄ perfluoroaryl, or
   heteroatom 3
selected from a group including a halogen atom and then the dashed line denotes a direct bond between the heteroatom and the R¹⁷ substituent, wherein the R¹⁷ substituent is a C₅-C₁₅ aryl, or a C₅-C₂₅ polyaryl, possibly substituted with 1-4 substituents selected independently from a group including a hydrogen atom, a halogen atom, a C₁-C₂₅ alkyl, a C₃-C₂₅ cycloalkyl, a C₂-C₂₅ alkenyl, a C₃-C₂₅ cycloalkenyl, a C₂-C₂₅ alkynyl, a C₃-C₂₅ cycloalkynyl, a C₁-C₂₅ perfluoroalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₆-C₂₀ heteroaryl or a C₅-C₂₄ heteroaryloxyl, 3-12-membered heterocycle, an alkoxy group (—OR″), a sulphide group (—SR″), a sulphoxide group (—S(O)R″), a sulphonium group (—S⁺R″₂), a sulphone group (—SO₂R″), a sulphonamide group (—SO₂NR″₂), an amine group (—NR″₂), an ammonium group (—N⁺R″₃), a nitro group (—NO₂), a cyano group (—CN), a phosphonous group (—P(O)(OR″)₂), a phosphinous group (—P(O)R″(OR″)), a phosphonin group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes a C₁-C₅ alkyl, a C₁-C₅ perfluoroalkyl, a C₆-C₂₄ aryl, a C₇-C₂₄ aralkyl, a C₅-C₂₄ perfluoroaryl.

The ruthenium complex is preferably represented by the formula 1a-Ru

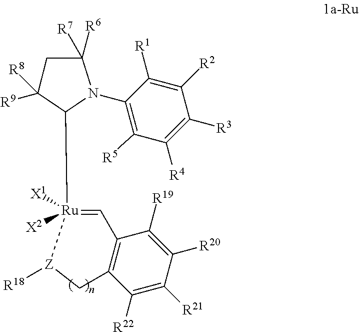

in which X¹ and X² and the R¹, R², R³, R⁴, R⁵, R⁶, R⁷, R⁸, R⁹ substituents have the meanings defined above
‘n’ means 1 or 0
Z is selected from a group including halogen atoms, 0 atom, S atom, Se atom, or a NR′″ group, in which R′″ denotes methylidene, a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl or a C₅-C₂₄ heteroaryloxyl, 3-12-membered heterocycle, an acyl group (—COR′), an ester group (—COOR′), a tert-butylcarboxycarbon group (t-Boc) or a 9-fluorenylmethoxycarbonyl group (Fmoc), a carbamine group (—CONR′₂), a sulphone group (—SO₂R′), a formyl group (—COH), in which the R′ group denotes a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl or a C₅-C₂₄ heteroaryloxyl,
or a halogen atom, wherein if Z denotes a halogen atom, R¹⁸ is absent;
R¹ means independently a halogen atom, a C₁-C₂₅ alkyl, a C₁-C₂₅ cycloalkyl, a C₅-C₂₀ alkoxyl, a C₅-C₂₀ aryl, a C₅-C₂₄ aryloxyl, a —COOR′″ group, a —CH₂COOR′″ group, a —CONR′″₂ group, a —CH₂CONR′″₂ group, a —COR′″ group, a —CH₂COR′″ group, a —CON(OR′″)(R′″) group, a —CH₂CON(OR′″)(R′″) group or a halogen atom, wherein R′″ denotes a C₁-C₁₂ alkyl, a C₂-C₁₂ cycloalkyl, a C₂-C₁₂ alkenyl, a C₆-C₂₀ aryl, which are possibly substituted with at least C₁-C₁₂ alkyl, a C₁-C₁₂perfluoroalkyl, a C₁-C₁₂ alkoxyl, a C₆-C₂₄ aryloxyl or a halogen atom;
R¹⁹, R²⁰, R²¹ and R²² denote independently a hydrogen atom, a halogen atom, a C₁-C₂₅ alkyl group, a C₂-C₂₅ alkenyl group, a C₅-C₂₅ aryl group, an alkoxy group (—OR″), a sulphide group (—SR″), a sulphoxide (—S(O)R″), a sulphonium group (—S⁺R″₂), a sulphone group (—SO₂R″), a sulphonamide group (—SO₂NR″₂), an amine group (—NR″₂), an ammonium group (—N⁺R″₃), a nitro group (—NO₂), a cyano group (—CN), a phosphonous group (—P(O)(OR″)₂), a phosphinous group (—P(O)R″(OR″)), a phosphonine group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R′), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes a C₁-C₅ alkyl, a C₁-C₅ perfluoroalkyl, a C₆-C₂₄ aryl, a C₇-C₂₄ aralkyl, a C5-C24 perfluoroaryl, wherein the R¹⁶, R¹⁷, R¹⁸ and R¹⁹ substituents may be connected, thus forming a substituted or an unsubstituted C₄-C₁₀ cyclic or a C₄-C₁₂ polycyclic system.

The ruthenium complex is preferably represented by the formula 1b-Ru

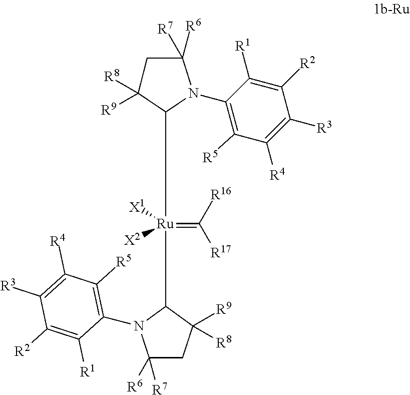

in which X¹ and X² and the R¹, R², R³, R⁴, R⁵, R⁶, R⁷, R⁸, R⁹ substituents have the meanings defined above
R¹⁶ and R¹⁷ independently denote a hydrogen atom, a halogen atom, a C₁-C₂₅ alkyl, possibly substituted, a C₃-C₂₅ cycloalkyl, possibly substituted, a C₁-C₁₂ perfluoroalkyl, possibly substituted, a C₂-C₂₅ alkene, possibly substituted, a C₂-C₂₅ alkenyl, possibly substituted, a C₃-C₂₅ cycloalkenyl, possibly substituted, a C₂-C₂₅ alkynyl, possibly substituted, a C₃-C₂₅ cycloalkynyl, possibly substituted, a C₁-C₂₅ alkoxyl, possibly substituted, a C₅-C₂₅ aryl, possibly substituted, a C₅-C₂₅ aryloxyl, possibly substituted, a C₆-C₂₅ arylalkyl, possibly substituted, a C₅-C₂₅ heteroaryl, possibly substituted, a C₅-C₂₅ heteroaryloxyl, possibly substituted, a C₅-C₂₅ perfluoroaryl, possibly substituted, a 3-12-membered heterocycle containing a sulphur, oxygen, nitrogen, selenium or phosphorus atom, possibly substituted;
wherein the R¹⁶ and R¹⁷ substituents may be connected, forming a ring selected from a group including a C₃-C₂₅ cycloalkyl, a C₃-C₂₅ cycloalkenyl, a C₃-C₂₅ cycloalkynyl, a C₅-C₂₅ aryl, a C₅-C₂₅ heteroaryl, a C₅-C₂₅ perfluoroaryl, a 3-12-membered heterocycle containing a sulphur, oxygen, nitrogen, selenium or phosphorus atom, which may be independently substituted with one and/or more substituents selected from a group including a hydrogen atom, a halogen atom, a C₁-C₂₅ alkyl, a C₃-C₂₅ cycloalkyl, a C₁-C₁₂ perfluoroalkyl, a C₂-C₂₅ alkene, a C₂-C₂₅ alkenyl, a C₃-C₂₅ cycloalkenyl, a C₂-C₂₅ alkynyl, a C₃-C₂₅ cycloalkynyl, a C₁-C₂₅ alkoxyl, a C₅-C₂₅ aryl, a C₅-C₂₅ aryloxyl, a C₆-C₂₅ arylalkyl, a C₅-C₂₅ heteroaryl, a C₅-C₂₅ heteoaryloxyl, a C₅-C₂₅ perfluoroaryl, a 3-12-membered heterocycle.

The ruthenium complex is preferably represented with the formula 1c-Ru or 1d-Ru or 1e-Ru

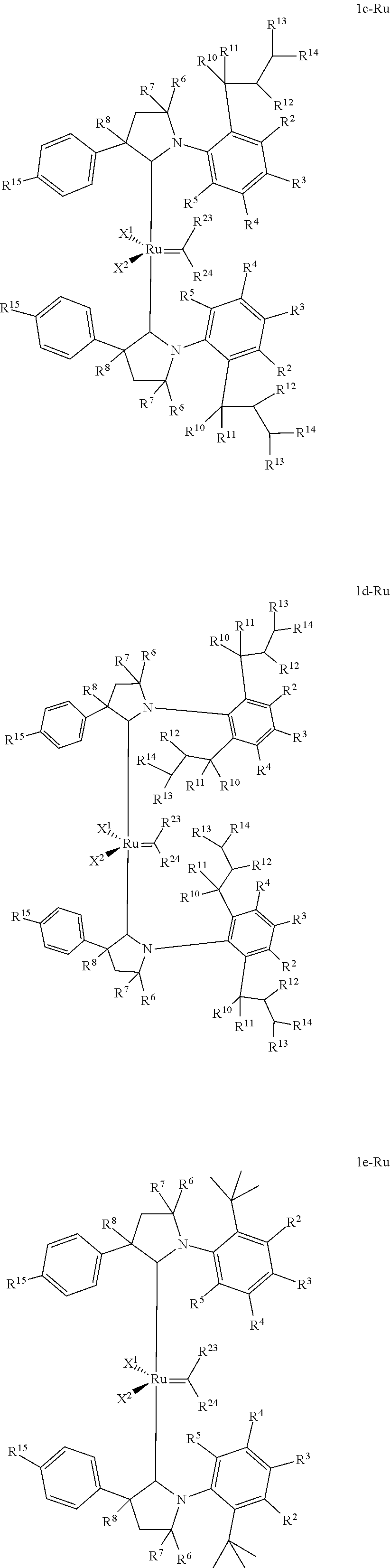

in which X¹ and X² as well as the R¹, R², R³, R⁴, R⁵, R⁶, R⁷, R⁸, R¹⁰, R¹¹, R¹², R¹³, R¹⁴, R¹⁵, R²³, R²⁴ substituents have the meanings defined above.

The ruthenium complex is preferably selected from complexes represented with the formulae Ru-a, Ru-b, Ru-c, Ru-d, Ru-e, Ru-f, Ru-g, Ru-h, Ru-i, Ru-j, Ru-k, Ru-l:

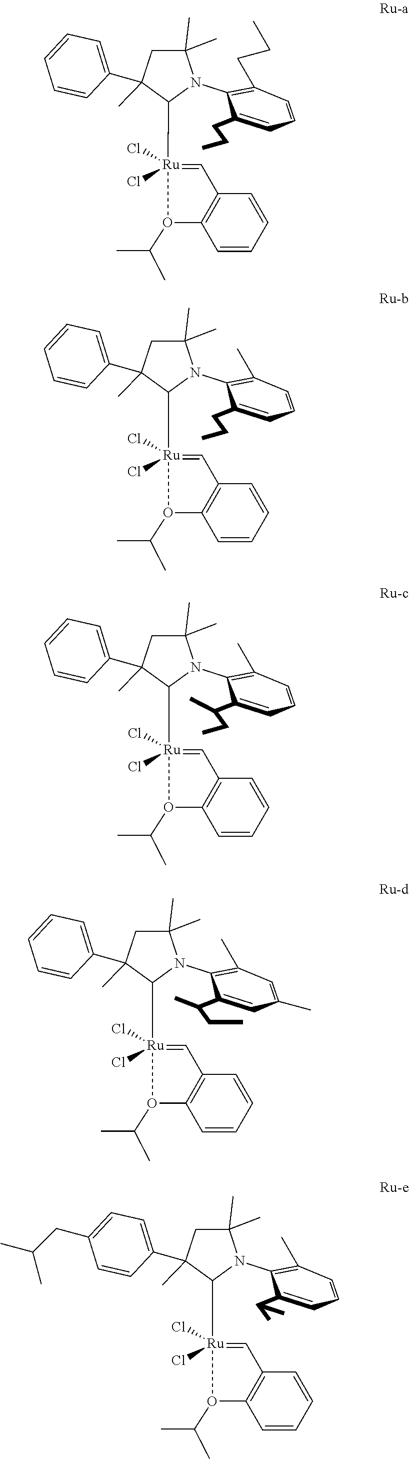

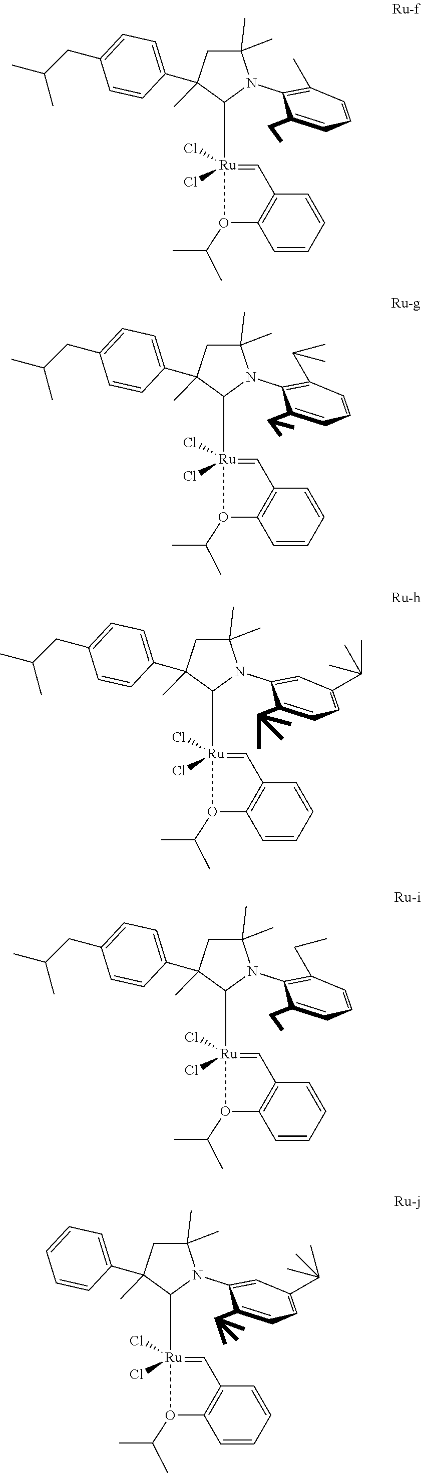

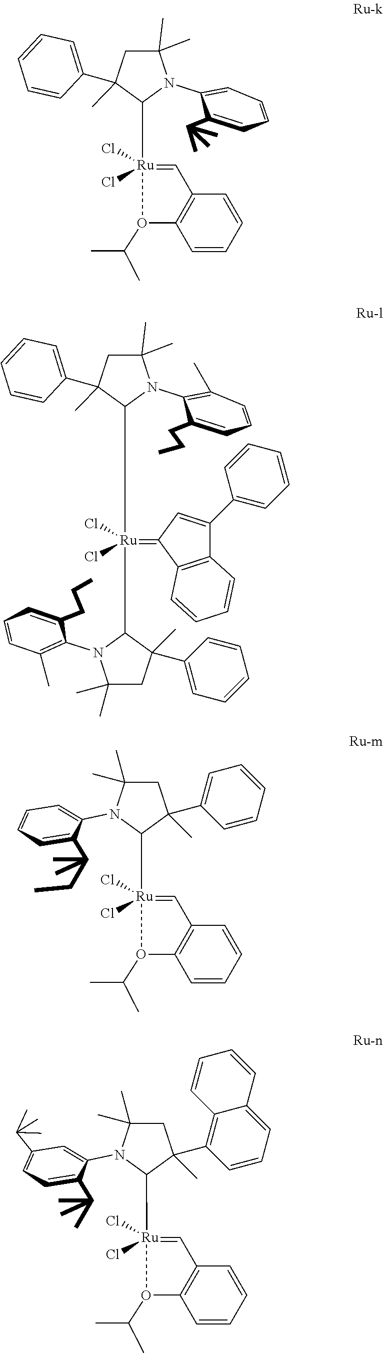

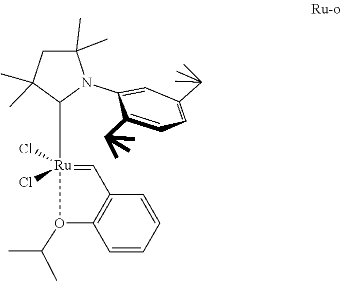

The invention is also related to a method of synthesis of the ruthenium complex with the formula 1-a-Ru

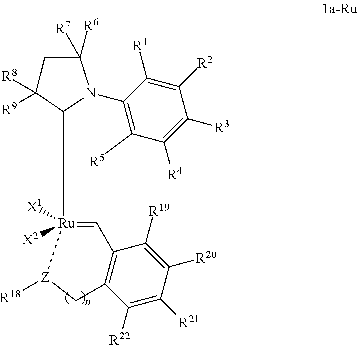

defined above, characterised in that the alkylidene ruthenium complex with the formula 10

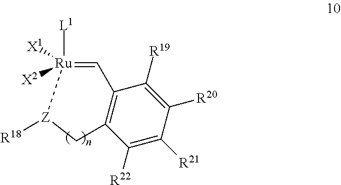

in which:
L¹ denote a neutral ligand selected from a group including pyridine or a substituted pyridine, P(R′)₃, P(OR′)₃, O(R′)₂, N(R′)₃, in which each R′ independently denotes a C₁-C₁₂ alkyl, a C₃-C₁₂ cycloalkyl, a C₅-C₂₀ aryl, a C₇-C₂₄ aralkyl, a C₅-C₂₄ perfluoroaryl, 5-12-membered heteroaryl;
N, Z, X¹, X² and the substituents R¹⁸, R¹⁹, R²⁰, R²¹ and R²² have the meanings defined above are subjected to a reaction with a carbene with the formula 8

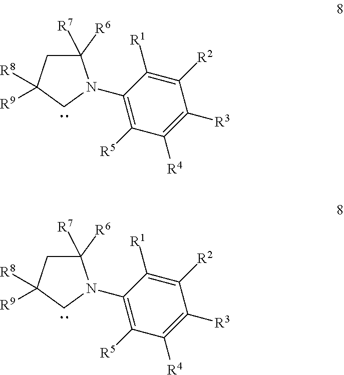

in which the substituents R¹ to R⁹ have the meanings defined above.

The subject of the invention also includes application of the compound with the formula 1-Ru, defined above, as a pre-catalyst and/or a catalyst in olefin metathesis reactions, in particular in ring closure metathesis (RCM) reactions, cross metathesis (CM), homometathesis (cross metathesis between two molecules of the same olefin), ethenolyse, isomerisation, in the reaction of diastereoselective ring rearrangement metathesis (DRRM), “alken-alkyn” (en-yn) type metathesis or a polymerisation reaction of the ROMP or ADMET type.

The reaction is preferably carried out in an organic solvent, such as toluene, mesitylene, hexane, cyclohexane, ethyl acetate, methyl acetate, methyl carbonate, ethyl carbonate, tert-butyl-methyl ether, cyclopentyl-methyl ether, diethyl ether, THF, 2-Me-THF, 4-Me-THP, dioxane, DME, PAO. PEG, paraffin, esters of saturated fatty acids.

The reaction is preferably carried out in a solvent-free system.

The reaction is preferably carried out at a temperature between 2° and 200° C.

The reaction is preferably carried out over a time between 5 minutes and 48 hours.

The 1-Ru compound is preferably used in a quantity not greater than 10% mol.

The 1-Ru compound is preferably used in a quantity not greater than 0.1% mol.

The 1-Ru compound is preferably added to the reaction mixture in aliquots, as a solid and/or continuously, using a pump, as a solution in an organic solvent.

The gaseous by-product of the reaction, selected from ethylene, propylene, butylene, is preferably actively removed from the reaction mixture using inert gas barbotage or under reduced pressure.

The subject of the invention also includes a ruthenium complex represented by the formula 1aa-Ru

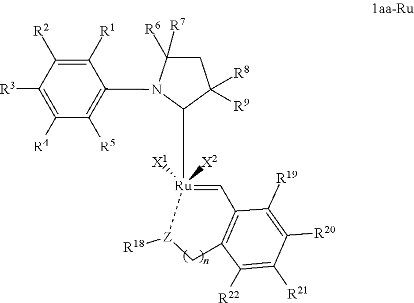

in which X¹ and X² independently from each other denote an anionic ligand selected from a group including a halogen anion, a —CN, —SCN, —OR^{a}, —SR^{a}, —O(C═O)R^{a}, —O(SO₂)R^{a} and —OSi(R^{a})₃ group, in which R^{a} denotes a C₁-C₁₂ alkyl, a C₃-C₁₂ cycloalkyl, a C₂-C₁₂ alkenyl or a C₅-C₂₀ aryl, which is optionally substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom;
R¹, R², R³, R⁴ and R⁵ denote independently a hydrogen atom, a C₁-C₁₂ alkyl group, a C3-C₂ cycloalkyl group, a C₅-C₂₀ aryl group or a C₅-C₂₀ heteroaryl group, a C₅-C₂₅ aralkyl group, which may be independently substituted by one and/or more substituents selected from a group including a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group, a sulphide group (—SR″), an amine group (—NR″₂), in which the R″ group independently denotes a hydrogen atom, C₁-C₅ alkyl, C₆-C₂₄ aryl, C₇-C₂₄ aralkyl, alternatively R¹, R², R³, R⁴ and R⁵ are connected and form a C₅-C₂₅ ring
wherein at least one substituent R¹, R², R³, R⁴ and R⁵ has an alkyl substituent with at least one quaternary carbon atom, preferably at least one substituent R¹, R⁴ and R⁵ has an alkyl substituent with at least two quaternary carbon atoms, more preferably at least one substituent R¹, R⁴ and R⁵ has an alkyl substituent with at least three quaternary carbon atoms,
each substituent R⁶ and R⁷ denotes a hydrogen atom, a halogen atom, a C₁-C₁₂ alkyl group or a C₅-C₂₀ aryl group, which may be substituted independently by one and/or more substituents selected from a group including hydrogen, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently means a hydrogen atom, a C₁-C₅ alkyl, a C₆-C₂₄ aryl, a C₇-C₂₄ aralkyl, alternatively, R⁶ and R⁷ and/or R⁸ and R⁹ are connected, forming a C₅-C₂₅ ring group.
the R⁸ substituent denotes a C₅-C₂₀ aryl group, which may be independently substituted with one and/or more substituent selected from a group including hydrogen, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently denotes a hydrogen atom, a C₁-C₅ alkyl, a C₆-C₂₄ aryl, a C₇-C₂₄ aralkyl,
the R⁹ substituent denotes a hydrogen atom, a halogen atom, a C₁-C₁₂ alkyl group, which may be independently substituted with one and/or more substituent selected from a group including hydrogen, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently denotes a hydrogen atom, a C₁-C₅ alkyl, a C₆-C₂₄ aryl, a C₇-C₂₄ aralkyl,
R⁸ and R⁹ are alternatively connected and form a C₅-C₂₅ ring group.
‘n’ means 1 or 0;
Z is selected from a group including halogen atoms, 0 atom, S atom, Se atom, or a NR′″ group, in which R′″ denotes methylidene, a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl or a C₅-C₂₄ heteroaryloxyl, 3-12-membered heterocycle, an acyl group (—COR′), an ester group (—COOR′), a tert-butylcarboxycarbon group (t-Boc) or a 9-fluorenylmethoxycarbonyl group (Fmoc), a carbamine group (—CONR′₂), a sulphone group (—SO₂R′), a formyl group (—COH), in which the R′ group denotes a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl or a C₅-C₂₄ heteroaryloxyl,
or a halogen atom, wherein if Z denotes a halogen atom, R¹⁸ is absent;
R¹⁸ means independently a halogen atom, a C₁-C₂₅ alkyl, a C₁-C₂₅ cycloalkyl, a C₅-C₂₀ alkoxyl, a C₅-C₂₀ aryl, a C₅-C₂₄ aryloxyl, a —COOR′″ group, a —CH₂COOR′″ group, a —CONR′″₂ group, a —CH₂CONR′″₂ group, a —COR′″ group, a —CH₂COR′″ group, a —CON(OR′″)(R′″) group, a —CH₂CON(OR′″)(R′″) group or a halogen atom, wherein R′″ denotes a C₁-C₂ alkyl, a C₃-C₂ cycloalkyl, a C₂-C₁₂ alkenyl, a C₆-C₂₀ aryl, which are possibly substituted with at least C₁-C₁₂ alkyl, a C₁-C₁₂perfluoroalkyl, a C₁-C₁₂ alkoxyl, a C₆-C₂₄ aryloxyl or a halogen atom;
R¹⁹, R²⁰, R²¹ and R²² denote independently a hydrogen atom, a halogen atom, a C₁-C₂₅ alkyl group, a C₂-C₂₅ alkenyl group, a C₅-C₂₅ aryl group, an alkoxy group (—OR″), a sulphide group (—SR″), a sulphoxide (—S(O)R″), a sulphonium group (—S⁺R″₂), a sulphone group (—SO₂R″), a sulphonamide group (—SO₂NR″₂), an amine group (—NR″₂), an ammonium group (—N⁺R″₃), a nitro group (—NO₂), a cyano group (—CN), a phosphonous group (—P(O)(OR″)₂), a phosphinous group (—P(O)R″(OR″)), a phosphonine group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R′), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes a C₁-C₅ alkyl, a C₁-C₅ perfluoroalkyl, a C₆-C₂₄ aryl, a C₇-C₂₄ aralkyl, a C5-C24 perfluoroaryl, wherein the R¹⁶, R¹⁷, R¹⁸ and R¹⁹ substituents may be connected, thus forming a substituted or an unsubstituted C₄-C₁₀ cyclic or a C₄-C₁₂ polycyclic system.

The aforementioned ruthenium complex is preferably represented by the formula 1aaa-Ru

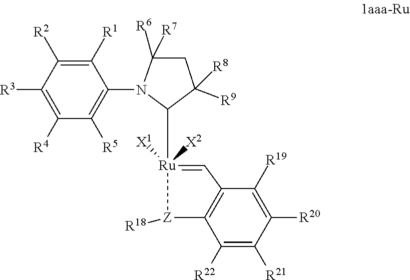

in which X¹ and X² and the R¹, R², R³, R⁴, R⁵, R⁶, R⁷, R⁸, R⁹ substituents have the meanings defined above;
Z is selected from a group including halogen atoms, 0 atom, S atom, Se atom.

The aforementioned ruthenium complex is preferably represented by the formula 1aaaa-Ru

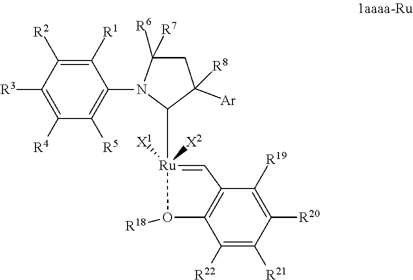

in which X¹ and X² and the R¹, R², R³, R⁴, R⁵, R⁶, R⁷, R⁸ substituents have the meanings defined above;
Ar denotes a C₆-C₂₀ aryl group, which may be independently substituted with one and/or more substituents selected from a group including hydrogen, a C₁-C₁₂ alkyl group, a C₁C2 perfluoroalkyl group, a C₅-C₂₀ aryl group, a C₅-C₂₀ perfluoroaryl group, a C₃-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently denotes a hydrogen atom, a C₁-C₅ alkyl, a C₆-C₂₄ aryl, a C₇-C₂₄ aralkyl,

Preferably, the ruthenium complex defined above, in which at least one substituent among R¹, R², R³, R⁴, R⁵ denotes a tert-butyl or tert-amyl substituent.

The subject of the invention is explained in embodiments presented in the figures, where:

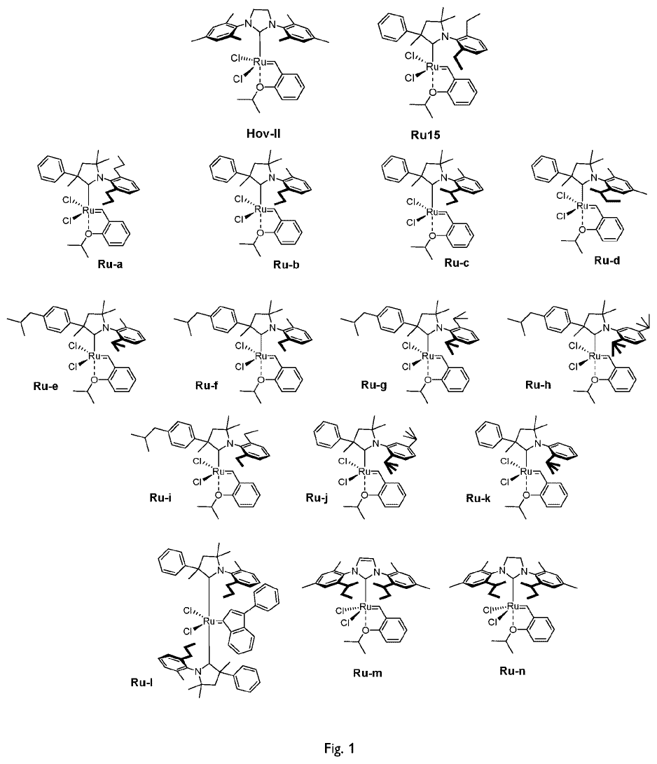

FIG. 1 presents a summary of precatalysts and catalysts of olefin metathesis available on the market, and of novel precatalysts and catalysts according to the invention;

FIG. 2 presents the structure of the Ru-a compound obtained on the basis of X-ray structural analysis;

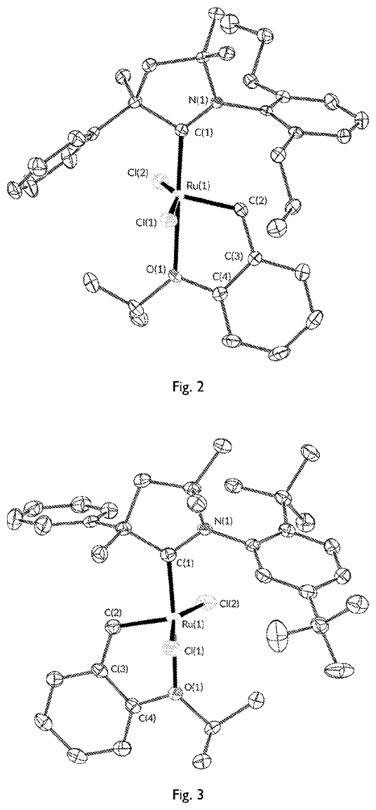

FIG. 3 presents the structure of the Ru-j compound obtained on the basis of X-ray structural analysis;

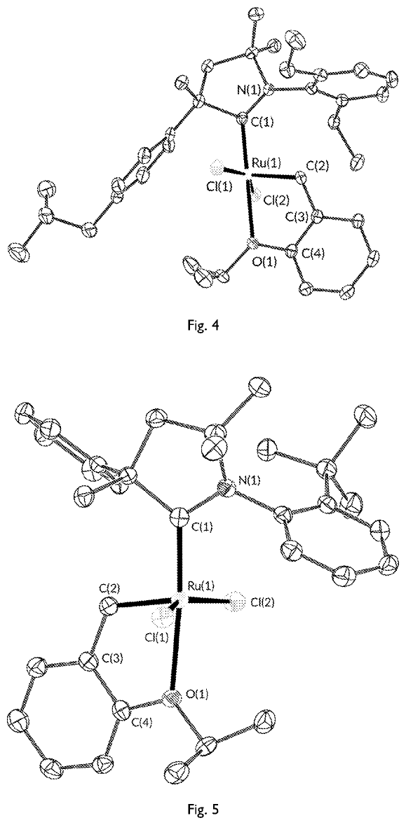

FIG. 4 presents the structure of the Ru-i compound obtained on the basis of X-ray structural analysis;

FIG. 5 presents the structure of the Ru-k compound obtained on the basis of X-ray structural analysis;

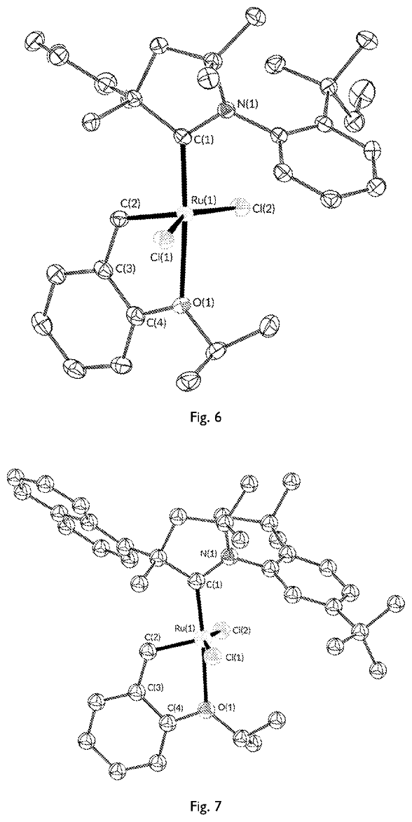

FIG. 6 presents the structure of the Ru-m compound obtained on the basis of X-ray structural analysis;

FIG. 7 presents the structure of the Ru-n compound obtained on the basis of X-ray structural analysis;

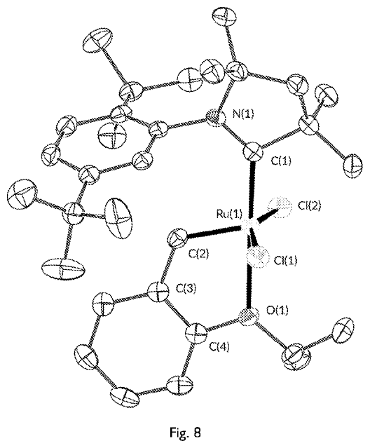

FIG. 8 presents the structure of the Ru-o compound obtained on the basis of X-ray structural analysis;

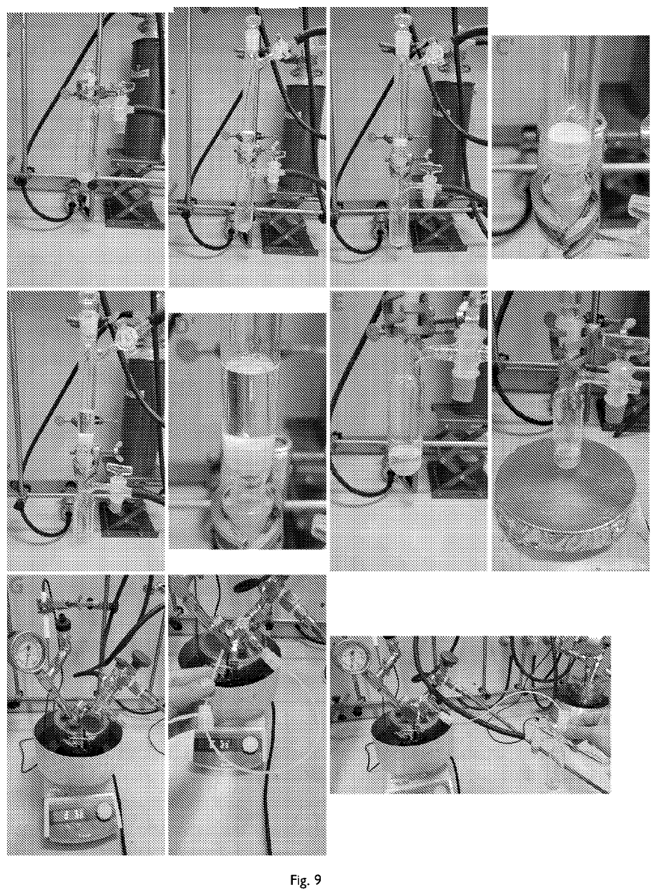

FIG. 9 equipment and procedure used to carry out the ethenolysis reaction. A—Schlenk tube with a magnetic stirrer bar attached to a Schlenk line, B—filtration funnel made of fritted glass connected to the Schlenk tube, C, C′—filtration funnel filled with Al₂O₃, D, D′—filtration funnel after filling with methyl oleate, E—Schlenk tube filled with filtered methyl oleate, F—methyl oleate degassing in vacuo, G—steel autoclave, H—Teflon tube connected to the autoclave, I—transfer of the mixture of methyl oleate and the catalysts to the autoclave through a Teflon tube using vacuum;

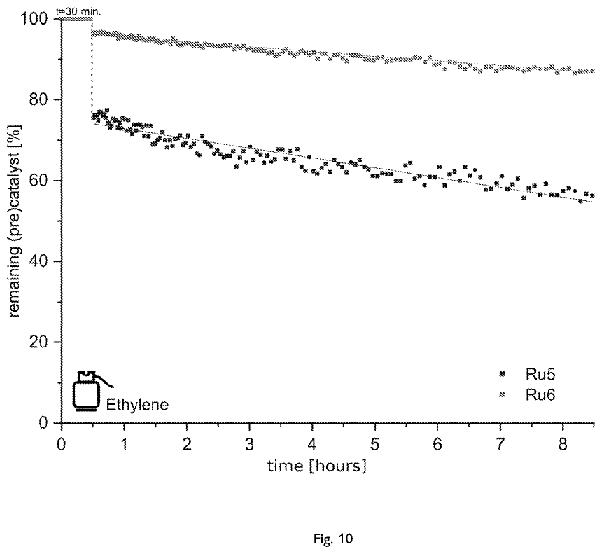

FIG. 10. General stability testing procedure of ruthenium complexes (Ru16 and Ru-k) in the presence of ethylene;

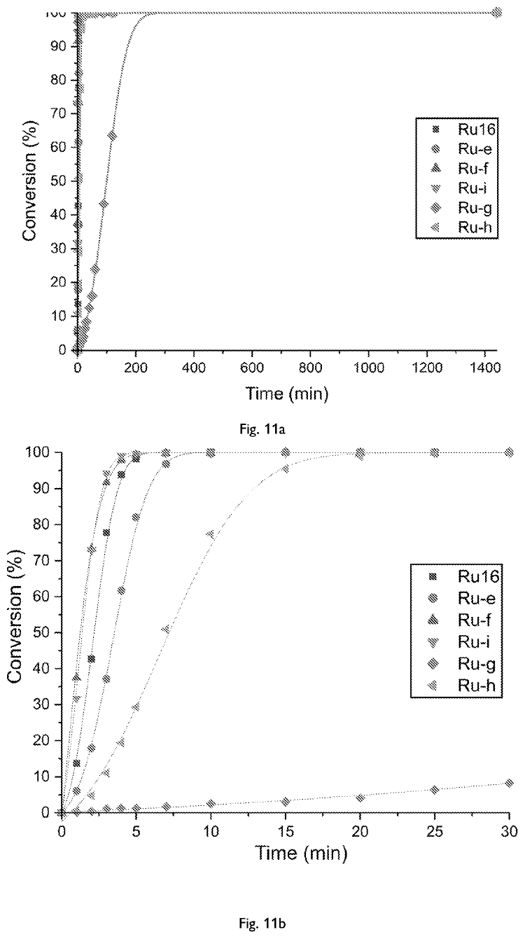

FIG. 11a graph with results for the RCM reaction (1,000 ppm of the ruthenium complex) of the DEDAM compound with trend lines, FIG. 11b graph with results for the RCM reaction (1,000 ppm of the ruthenium complex) of the DEDAM compound with trend lines (only the first 30 min);

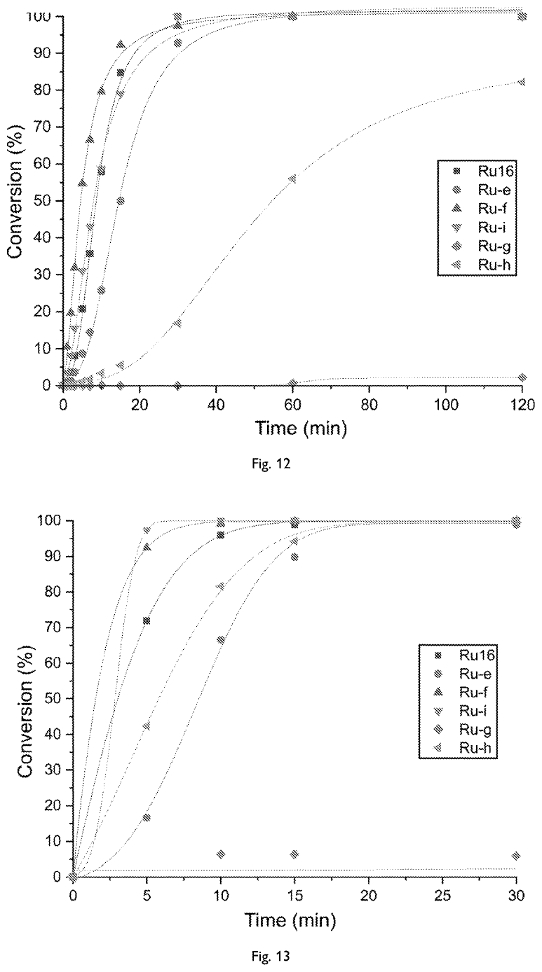

FIG. 12 graph with results for the RCM reaction (100 ppm of the ruthenium complex) of the DEDAM compound with trend lines;

FIG. 13 graph with results for the RCM reaction for the compound N-allyl-4-methyl-N-(2-metylallyl)benzenesulphonamide (1,000 ppm of the ruthenium complex) and trend lines (only the first 30 minutes);

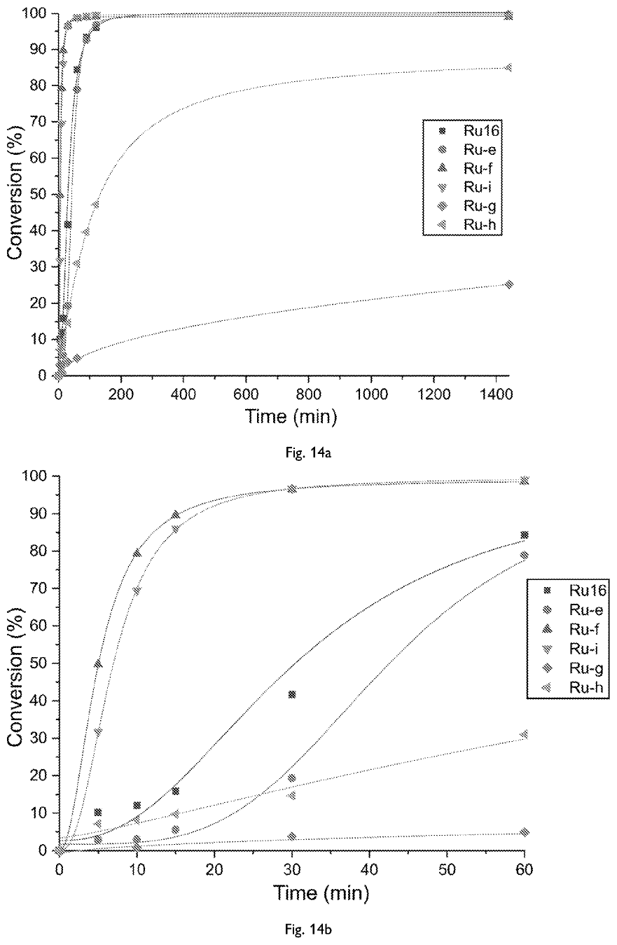

FIG. 14a graph with results for the RCM reaction for the compound N-allyl-4-methyl-N-(2-metylallyl)benzenesulphonamide (100 ppm of the ruthenium complex) and trend lines (only the first 60 minutes); FIG. 14a graph with results for the RCM reaction for the compound N-allyl-4-methyl-N-(2-metylallyl)benzenesulphonamide (100 ppm of the ruthenium complex) and trend lines (only the first 60 minutes);

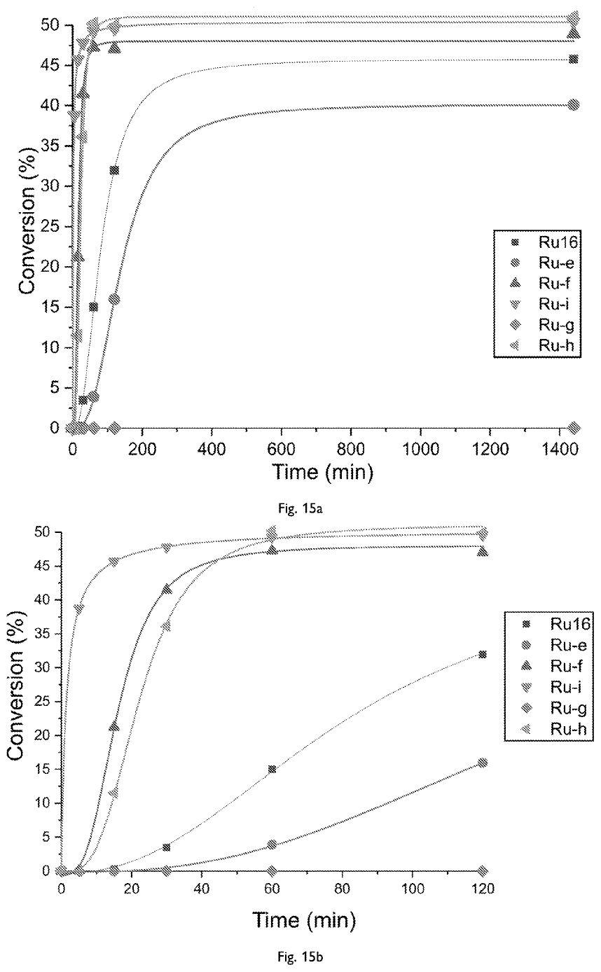

FIG. 15a graph with results for the SCM reaction of methyl oleate (2.5 ppm) and trend lines (only the first 120 minutes);

FIG. 15b graph with results for the SCM reaction of methyl oleate (2.5 ppm) and trend lines;

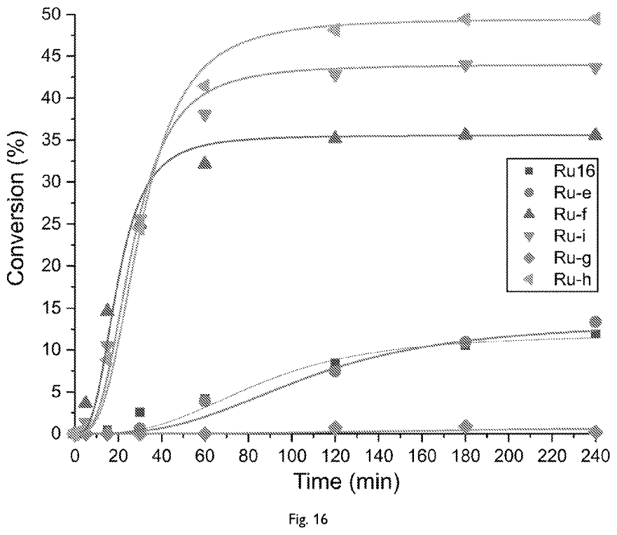

FIG. 16 graph with results for the self-CM reaction of methyl oleate (1.0 ppm) and trend lines;

Terms used in this disclosure have the following meanings.

Terms undefined in this document have meanings which are provided and understood by a specialist in the field in view of the best held knowledge, this disclosure and the context of the patent application disclosure. Unless specified otherwise, the following conventions of chemical terms with meanings as provided in the following definitions have been used: The term “halogen atom” used in this disclosure means an element selected from F, Cl, Br, I.

The term “carbene” means an electrically neutral molecule, in which the carbon atom has two non-binding electrons in a singlet or a triplet state and which is connected via a single covalent bond with two groups or connected via a double covalent bond with a single group. The term “carbene” also includes carbene analogues, in which the carbene carbon atom is replaced with a different chemical element, such as boron, silicon, germanium, tin, lead, nitrogen, phosphorus, sulphur, selenium or tellurium.

The term “olkyl” refers to a saturated, linear or branched hydrocarbon substituent with the indicated number of carbon atoms. Examples of alkyl substituents include -methyl, -ethyl, -n-propyl, -n-butyl, -n-pentyl, -n-hexyl, -n-heptyl, -n-octyl, -n-nonyl and -n-decyl. Representative branched —(C1-C10) alkyl include -isopropyl, -sec-butyl, -isobutyl, -tertbutyl, -isopentyl, -neopentyl, -1-methylbutyl, -2-methylbutyl, -3-methylbutyl, -1,1-dimethylpropyl, -1,2-dimethylpropyl, -1-methylpentyl, -2-methylpentyl, -3-methylpentyl, -4-methylpentyl, -1-ethylbutyl, -2-ethylbutyl, -1,1-dimethylbutyl, -1,2-dimethylbutyl, -1,3-dimethylbutyl, -2,2-dimethylbutyl, -2,3-dimethylbutyl, -3,3-dimethylbutyl, -1-methylhexyl, -2-methylhexyl, -3-methylhexyl, -4-methylhexyl, -1,2-dimethylpentyl, -1,3-dimethylpentyl, -5-methylhexyl, -1,2-dimethylhexyl, -1,3-dimethylhexyl, -3,3-dimethylhexyl, -1,2-dimethylheptyl, -1,3-dimethylheptyl, -3,3-dimethylheptyl and the likes.

The term “alkoxyl” refers to an alkyl substituent as defined above, connected via an oxygen atom.

The term “perfluoroalkyl” denotes an alkyl group as defined above, in which all hydrogen atoms have been replaced with the same or different halogen atoms.

The term “cycloalkyl” refers to a saturated, mono- or polycyclic hydrocarbon substituent with the indicated number of carbon atoms. Examples of cycloalkyl substituent include -cyclopropyl, -cyclobutyl, -cyclopentyl, -cyclohexyl, -cycloheptyl, -cyclooctyl, -cyclononyl, -cyclodecyl and the likes.

The term “alkenyl” refers to an unsaturated, linear or branched, acyclic hydrocarbon substituent with the indicated number of hydrogen atoms and containing at least one double carbon-carbon bond. Examples of alkenyl substituents include -vinyl, -allyl, -1-butenyl, -2-butenyl, -isobutenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the likes.

The term “cycloalkenyl” refers to an unsaturated, cyclic or branched, cyclic hydrocarbon substituent with the indicated number of hydrogen atoms and containing at least one double carbon-carbon bond. Examples of cycloalkenyl substituents include -cycloprope-ne, -cyclobutene, -cyclopentene, -cyclohexene, -cycloheptene, -cyclooctene, -cyclononene, -cyclo decene, -metylcyclopropene, -ethylcyclobutene, -isopropylcyclopentene, -methylcyclohexene and the likes.

The term “aryl” refers to an aromatic, mono- or polycyclic hydrocarbon substituent with the indicated number of carbon atoms. Examples of aryl substituents include -phenyl, -tolyl, -xylil, -naphthtyl, -2,4,6-trimethylphenyl, -2-fluorophenyl, -4-fluorophenyl, -2,4,6-trifluorophenyl, -2,6-difluorofenyl, -4-nitrofenyl and the likes.

The term “aralkyl” refers to an alkyl substituent as defined above, substituted with at least one aryl as defined above. Examples of aralkyl substituents include -benzyl, -diphenylmethyl, -triphenylmethyl and the likes.

The term “heteroaryl” refers to an aromatic mono- or polycyclic hydrocarbon substituent with the indicated number of carbon atoms, in which at least one carbon atom has been replaced with a heteroatom selected from O, N and S atoms. Examples of heteroaryl substituents include -furyl, -thienyl, -imidazolyl, -oxazolyl, -thiazolyl, -isoxazolyl, -triazolyl, -oxadiazolyl, -thiadiazolyl, -tetrazolyl, -pyridyl, -pyrimidyl, -triazinyl, -indolyl, -benzo[b]furyl, -benzo[b]thienyl, -indazolyl, -benzoimidazolyl, -azaindolyl, -quinolyl, -isoquinolyl, -carbazolyl and the likes.

The term “heterocycle” refers to a saturated, unsaturated or partially unsaturated hydrocarbon substituent, with the indicated number of carbon atoms, in which at least one carbon atom has been replaced with a heteroatom selected from O, N and S atoms. Example heterocycle substituents include -furyl, -thiophenyl, -pyrolyl, -oxazolyl, -imidazolyl, -thiazolyl, -isoxazolyl, -pyra-zolyl, -isothiazolyl, -triazinyl, -pyrolidinonyl, -pyrolidinyl, -hydantoinyl, -oxiranyl, -oxetanyl, -tetrahy drofuranyl, -tetrahydrothiophenyl, -quinolinyl, -isoquinolinyl, -chromonyl, -coumarinyl, -indolyl, -in dolysinyl, -benzo[b]furanyl, -benzo[b]thiophenyl, -indazolyl, -purinyl, -4H-quinolysinyl, -isoquinolyl, -quinolyl, -phtalasinyl, -naphtyridinyl, -carbazolyl, -β-carbolynyl and the likes.

The term “neutral ligand” refers to an uncharged substituent able to coordinate to a metal centre (a transition metal atom). Examples of such ligands may include: N-heterocyclic carbenes (NHC), cyclic (alkyl) (amine) carbenes (CAAC), amines, phosphines and their oxides, alkyl and aryl phosphites and phosphates, arsines and their oxides, ethers, alkyl and aryl sulphides, coordinated unsaturated or aromatic hydrocarbons, alkyl and aryl halides, nitriles, isonitriles, sulphides, sulphoxides, sulphones, thioketones, thioamides, thioester, thionoesters and dithioesters.

The term “anionic ligand” refers to a substituent able to coordinate to a charged metal centre (transition metal atom), which can partially or fully compensate the charge of the metal centre. Examples of such ligands may include fluoride, chloride, bromide, iodide, cyanide, cyanate and thiocyanate anions, carboxylic acid anions, alcohol anions, phenol anions, thiol and thiophenol anions, anions of hydrocarbons with delocalised charge (e.g. cyclopentadiene anion), anions of (organo)sulphuric acids and (organo)phosphoric acids and their esters (such as e.g. anions of alkylsulphonic and arylsulonic acids, anions of alkylphosphoric and arylphosphoric acids, anions of alkyl and aryl esters of sulphuric acid, anions of alkyl and aryl esters of phosphoric acids, anions of alkyl and aryl esters of alkylphosphoric and arylphosphoric acids).

The term “heteroatom” means an atom selected from the group including oxygen sulphur, nitrogen, phosphorous, boron, silicon, arsenic, selenium, tellurium.

### EXAMPLE EMBODIMENTS OF THE INVENTION

The following examples have been provided only to illustrate the invention and to explain its individual aspects and not to limit the invention and should not be interpreted as its entire scope, which is defined in the attached claims. Unless indicated otherwise, the following examples use standard materials and methods used in the art or the manufacturer recommendations for the specified reagents and methods were followed.

If necessary, model compounds for metathesis reaction were purified using fractional distillation and subsequently stored under an inert gas atmosphere, over activated, neutral aluminium oxide. Tetrahydrofuran was purified by distillation over a sodium-potassium alloy in the presence of benzophenone and subsequently stored over 4 Å molecular sieves. If justified, the selected reactions were carried out under argon atmosphere, using reaction vessels heated at 130° C. Aluminium oxide (Al₂O₃, neutral, Broockman Grade 1) was activated by heating at 150° C. under reduced pressure, for 16 hours.

The starting compounds for synthesis of novel aniline derivatives were available commercially.

### Example I

Synthesis of Novel Anilines with Branched Alkyl Substituents Using the Aza-Claisen Method

The Diagram 1 above illustrates the synthesis of CAAC ligand precursors enabling ruthenium catalysts of olefin metathesis to be obtained (general formula from Ru-a to Ru-l, FIG. 1), comprising the subject of this invention.

Reactions R-1 to R-2, R-1′ to R-3′ and R-1″ to R-3″ presented in Diagram 1 were carried out using commercially available substrates, according to procedures described in the literature, including change developed by the authors. Unless indicated otherwise, the described reactions used commercially available solvents and no regard to the presence of oxygen and/or moisture was paid. The individual transformations described in Diagram 1 are presented below.

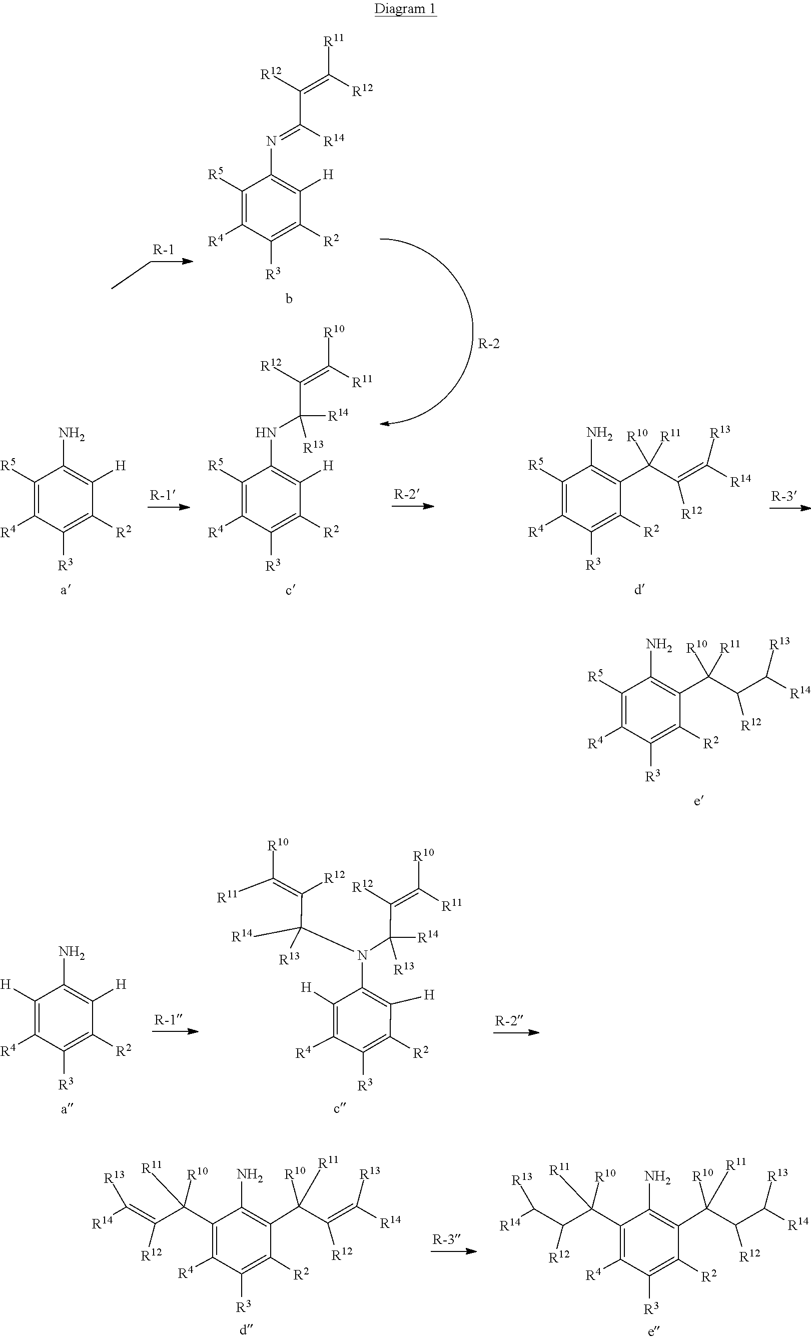

### Reaction R-1 to R-2

Synthesis of imine with the general formula b is carried out in the R-1 stage (Diagram 1). Aniline with general formula a′ and aldehyde, preferably crotonaldehyde, are used for this purpose. The transformation is preferably carried out in methylene chloride (DCM) or other organic solvent, using a stoichiometric amount of a drying agent, preferably magnesium sulphate (MgSO₄). The reaction is carried out at room temperature (RT). The product is separated from the reaction mixture through filtration and solvent distillation.

During the R-2 stage, the crude product is dissolved in an organic solvent, preferably methanol (MeOH) and a reducing agent is added, preferably sodium borohydride (NaBH₄) at 0° C. The reaction is carried out at room temperature, under argon atmosphere, for 16 hours. The product is separated through extraction from a water/n-hexane mixture.

### Example Embodiment from R-1 to R-2

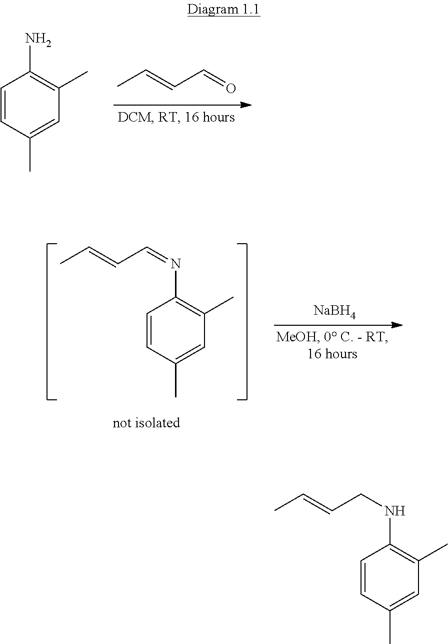

To a reaction vessel containing a stirring bar, 2,4-dimethylaniline (39.5 g, 0.33 mol, 1.0 equiv.), crotonaldehyde (34.4 g, 0.49 mol, 1.5 equiv.), 330 mL DCM and anh. MgSO₄ (49.2 g, 0.41 mol, 1.25 equiv.) were introduced. The contents of the vessel were stirred for 16 hours at room temperature. At the end of the reaction, the mixture was filtered through neutral Celite, the solvent was evaporated under reduced pressure and the expected product was obtained, which was used in the next stage without purification.

To the reaction vessel containing a stirring bar, imine from the previous stage and 500 mL MeOH were introduced under argon. To the reaction-wise, NaBH₄ was added in portions at 0° C. The reaction was carried out at 16 hours at room temperature. The solvent was evaporated under reduced pressure. Next, the mixture was dissolved in n-hexane, transferred to a separator, water was added and extracted thrice using n-hexane. The combined organic layers were dried over anh. MgSO₄, filtered through a layer of neutral Celite and the solvent was evaporated under reduced pressure. This resulted in the expected product in the form of a brown liquid, in 78% yield (45.1 g, 0.28 mol).

¹H NMR (400 MHz, CDC₃) δ ppm: 6.97-6.93 (m, 1H), 6.93-6.90 (m, 1H), 6.56 (d, J=8.1 Hz, 1H), 5.80-5.60 (m, 2H), 3.75-3.70 (m, 2H), 3.42 (bs, 1H), 2.25 (s, 3H), 2.14 (s, 3H), 1.74 (dq, J=6.2, 1.3 Hz, 3H).

¹³C NMR (100 MHz, CDC₃) δ ppm: 144.0, 131.0, 128.5, 127.9, 127.4, 126.2, 122.3, 110.3, 46.4, 20.5, 17.9, 17.6.

### Reaction R-1′

Synthesis of imine with the general formula c′ is carried out in the R-1′ stage (Diagram 1). Aniline with general formula a′ and alkyl halide, preferably bromide, chloride or iodide, are used for this purpose. The transformation is preferably carried out in N,N-dimethylformamide (DMF) or in a different organic solvent. The reaction mixture is kept at room temperature. The product is separated from the reaction mixture through filtration, solvent distillation and column chromatography [Chem. Commun., 2013, 49, 4346].

### Example Embodiment R-1′

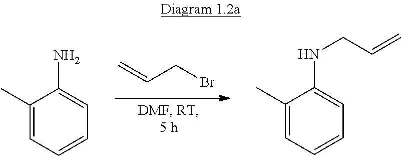

To a reaction vessel equipped with a stirring bar, containing a solution of o-toluidine (15.0 g, 14.9 mL, 0.14 mol, 2 equiv.) in DMF (60 mL) allyl bromide (8.7 g, 6.2 mL, 0.07 mol, 1 equiv.) was added dropwise at room temperature. The contents of the flask was stirred at room temperature for 5 hours. The reaction was monitored using thin layer chromatography (TLC). At the end of the reaction, water (50 mL) and ethyl acetate (EtOAc) (50 mL), were added to the mixture, stirred for 5 minutes and the layers were separated. The water layer was extracted with EtOAc (2×25 mL). The combined organic layers were washed with water (2×30 mL) to remove traces of DMF, water residues were dried over anhydrous sodium sulphate (Na₂SO₄), filtered, the solvent was evaporated under reduced pressure. The crude product was purified using column chromatography and n-hexane:EtOAc (99:1) as the eluent, and light yellow oil was obtained in 59% yield (6.1 g, 41 mmol).

¹H NMR (400 MHz, CDCl₃) δ ppm 7.22-7.15 (m, 1H), 7.15-7.08 (m, 1H), 6.73 (td, J=7.4, 1.2 Hz, 1H), 6.67 (d, J=8.0 Hz, 1H), 6.11-6.00 (m, 1H), 5.35 (dq, J=17.2, 1.7 Hz, 1H), 5.24 (dq, J=10.3, 1.5 Hz, 1H), 3.88 (d, J=5.6, Hz, 2H), 3.67 (s, 1H), 2.21 (s, 3H),

¹³C NMR (100 MHz, CDCl₃) δ ppm: 146.0, 135.6, 130.1, 127.1, 122.0, 117.1, 116.2, 110.0, 46.5, 17.5,

### Reaction R-1′—Alternative Method

Synthesis of imine with the general formula c′ is carried out in the R-1′ stage (Diagram 1). Aniline with general formula a′ and alkyl halide, preferably bromide, chloride or iodide, are used for this purpose. The transformation is preferably carried out in acetonitrile (MeCN) or a different organic solvent, in the presence of an inorganic salt, preferably potassium carbonate (K₂CO₃). The reaction is carried out at 60° C. The product is separated from the reaction mixture through filtration, solvent distillation and column chromatography [Angew. Chem. Int. Ed. 2019, 58, 4700]

### Alternative Embodiment R-1′

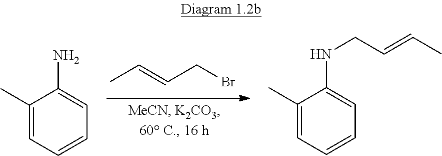

To a reaction vessel equipped with a stirring bar, containing a solution of o-toluidine (18.8 g, 18.6 mL, 0.14 mol, 3.5 equiv.) in acetonitrile (MeCN) (50 mL) crotyl bromide (8.1 g, 6.2 mL, 0.05 mol, 1 equiv.) was added dropwise at room temperature and K₂CO₃ (14 g, 0.10 mol, 2 equiv.). The reaction mixture was heated at 60° C. for 16 hours. The reaction was monitored using TLC. At the end of the reaction, water (50 mL) and diethyl ether (Et₂O) (50 mL) were added to the reaction mixture and it was extracted using diethyl ether (2×20 mL), the organic layer was washed with water (2×20 mL), dried over anhydrous Na₂SO₄, filtered and the solvent was evaporated under reduced pressure. The crude product was purified using column chromatography on silica gel, using n-hexane as eluent, and light yellow oil was obtained in 45% yield (3.6 g, 22.3 mmol). [Chem. Commun., 2013, 49, 4346-4348]

¹H NMR (400 MHz, CDCl₃) δ ppm: 7.22-7.20 (m, 2H), 6.75-6.63 (m, 2H), 5.86-5.45 (m, 2H), 3.90-3.72 (m, 2H), 3.54 (s, 1H), 2.18 (s, 3H), 1.77 (m, 3H).

¹³C NMR (100 MHz, CDCl₃) δ ppm: 146.2, 130.1, 128.2, 128.0, 127.2, 122.0, 117.0, 110.0, 46.0, 17.9, 17.6.

### Reaction R-2′

An aza-Claisen rearrangement leading to an aniline with the general formula d′ is carried out in the R2′ stage (Diagram 1). Aniline with general formula c′ and a Lewis acid, preferably boron trifluoride etherate, are used for this purpose. The reaction is preferably carried out in chlorobenzene, o-xylene or other organic solvent. The reaction is carried out at reflux. The product was purified through filtration, solvent distillation and column chromatography. [Chem. Commun., 2013, 49, 4346].

### Example Embodiment R-2′

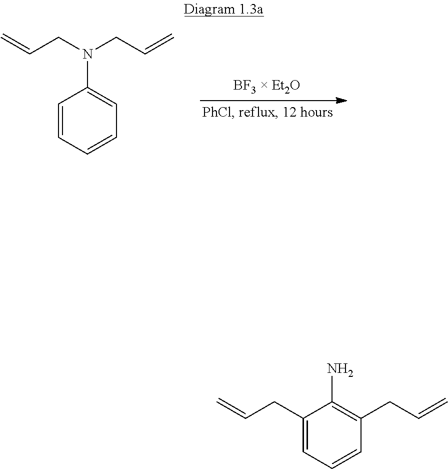

To a reaction vessel equipped with a stirring bar, containing a solution of N-allyl-2-methylaniline (4.12 g, 28 mmol, 1 equiv.) in chlorobenzene (PhCl) (25 mL) boron trifluoride etherate (6.0 g, 5.2 mL, 42 mmol, 1.5 equiv.) was slowly added dropwise, and the mixture was heated at reflux for 12 hours. Next, the mixture was cooled to room temperature and saturated solution of sodium bicarbonate (NaHCO₃) (20 mL) was added, the aqueous layer was extracted with EtOAc (2×25 mL), the combined organic fractions were washed with water (25 mL), brine (25 mL), and then dried over Na₂SO₄, filtered, the solvent was evaporated. The crude product was purified using column chromatography, using n-hexane as eluent, and light yellow oil was obtained in 56% yield (2.3 g, 15.6 mmol).

¹H NMR (400 MHz, CDCl₃) δ ppm: 7.01 (d, J=7.5, Hz, 1H), 6.97 (d, J=7.5, Hz, 1H), 6.72 (t, J=7.5 Hz, 1H), 6.07-5.92 (m, 1H), 5.21-5.09 (m, 2H), 3.68 (s, 2H), 3.36 (dt, J=6.2, 1.7 Hz, 2H), 2.21 (s, 3H).

¹³C NMR (100 MHz, CDCl₃) δ ppm 143.0, 136.1, 128.8, 128.0, 123.3, 122.4, 118.2, 116.1, 36.8, 17.7.

### Alternative Embodiment R-2″

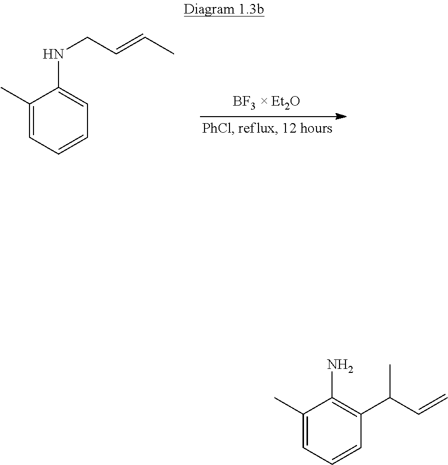

To a reaction vessel equipped with a stirring bar, containing a solution of N-2-butenyl-2-methylaniline (2.33 g, 14.4 mmol, 1 equiv.) in PhCl (25 mL) boron trifluoride etherate (2.7 mL, 21.6 mmol, 1.5 equiv.) was slowly added dropwise, and the mixture was heated at reflux for 12 hours. Next, the mixture was cooled to room temperature and saturated solution of sodium bicarbonate (NaHCO₃) (20 mL) was added, the aqueous layer was extracted with EtOAc (2×25 mL), the combined organic fractions were washed with water (25 mL), brine (25 mL), and then dried over Na₂SO₄, filtered, the solvent was evaporated. The crude product was purified using column chromatography, using n-hexane as eluent, and light yellow oil was obtained in 42% yield (0.98 g, 6.08 mmol).

¹H NMR (400 MHz, CDCl₃) δ ppm: 7.04-6.94 (m, 2H), 6.76 (t, J=7.5 Hz, 1H), 6.03-5.88 (m, 1H), 5.15-5.04 (m, 2H), 4.55-4.08 (brs, 2H), 3.60-3.48 (m, 1H), 2.22 (s, 3H), 1.41 (d, J=7.0 Hz, 3H).

¹³C NMR (100 MHz, CDCl₃) δ ppm: 142.3, 128.6, 127.9, 126.7, 125.0, 123.1, 118.9, 113.8, 38.3, 18.9, 17.9.

### Alternative Embodiment R-2″

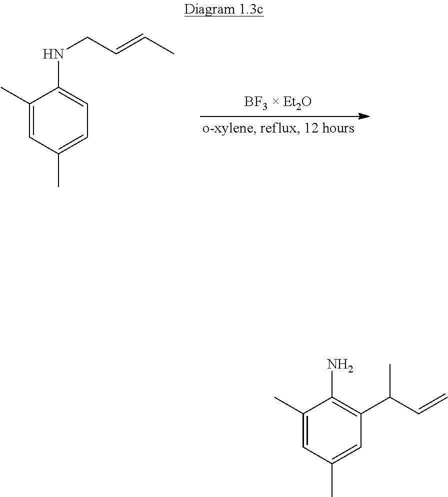

To a reaction vessel equipped with a stirring bar, containing a solution of N-2-butenyl-2,4-dimethylaniline (54.3 g, 0.26 mol, 1 equiv.) in o-xylene (467 mL) boron trifluoride etherate (41.3 mL, 0.33 mol, 1.3 equiv.) was slowly added dropwise, and the mixture was heated at reflux for 12 hours. Next, the mixture was cooled to room temperature and saturated NaHCO₃ solution was added, the aqueous layer was extracted with EtOAc, the combined organic fraction were washed, in turns, with water, brine and subsequently dried over Na₂SO₄, filtered, the solvent was evaporated. The crude product was purified by distillation, obtaining a colourless oil in >99% yield (45 g, 0.26 mol).

¹H NMR (400 MHz, CDCl₃) δ ppm: 6.82 (d, J=0.7 Hz, 2H), 6.02-5.92 (m, 1H), 5.13 (dt, J=6.5, 1.6 Hz, 1H), 5.11-5.09 (m, 1H), 3.58 (bs, 2H), 3.54-3.43 (m, 1H), 2.25 (s, 3H), 2.18 (s, 3H), 1.41 (d, J=7.0 Hz, 3H).

¹³C NMR (100 MHz, CDCl₃) δ ppm: 142.6, 140.0, 129.3, 128.5, 127.4, 125.6, 122.8, 113.8, 38.5, 20.7, 19.0, 17.9.

### Reaction R-3′

A hydrogenation reaction of unsaturated C═C bonds leading to an aniline with the general formula e′ is carried out in the R-3′ stage (Diagram 1). Aniline with general formula d′ and a catalyst, preferably Pd/C and gaseous hydrogen, are used for this purpose. The reaction is preferably carried out in methanol or other organic solvent. The reaction is carried out at room temperature. The product is separated from the reaction mixture through filtration, solvent distillation and distillation or column chromatography.

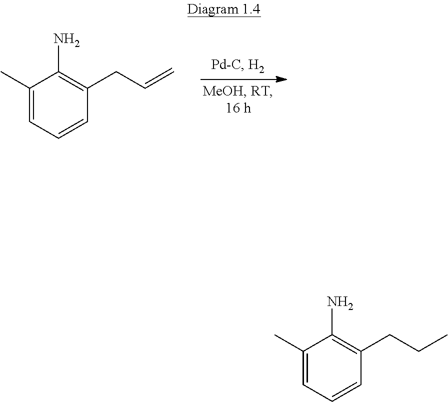

### Example Embodiment R-3′

To a reaction vessel equipped with a stirring bar, containing a solution of aniline (1.91 g, 13 mmol, 1 equiv.) in MeOH (50 mL), Pd/C (0.14 g, 0.13 mmol, 0.01 equiv., 10% wt.) was added. The reaction flask was purged with hydrogen and the reaction was carried out at room temperature for 16 hours, in a H₂ atmosphere (1 atm.). Next, the reaction mixture was filtered through Celite and the filtrate was concentrated. The crude product was purified by distillation under reduced pressure. Yellow oil was obtained in 89% yield (1.73 g, 11.6 mmol). [Eur. J. Med. Chem., 2019, 176, 162-174]

¹H NMR (400 MHz, CDCl₃) δ ppm: 6.95 (d, J=7.5, Hz, 2H), 6.68 (t, J=7.5 Hz, 1H), 3.72 (brs., 2H), 2.50 (t, J=7.5, Hz, 2H), 2.20 (s, 3H), 1.72-1.61 (m, 2H), 1.02 (t, J=7.3 Hz, 3H).

¹³C NMR (100 MHz, CDCl₃) δ ppm: 142.1, 128.2, 127.3, 126.2, 122.2, 118.1, 33.7, 21.9, 17.8, 14.3.

### Alternative Embodiment R-3′

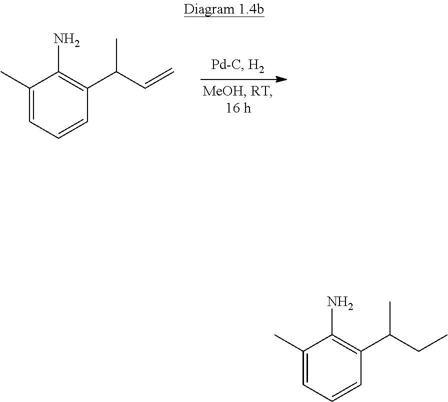

To a reaction vessel equipped with a stirring bar, containing a solution of aniline (0.8 g, 5 mmol, 1 equiv.) in MeOH (30 mL), Pd/C (0.05 g, 0.05 mmol, 0.01 equiv., 10% wt.) was added. The reaction flask was purged with hydrogen and the reaction was carried out at room temperature for 16 hours, in a H₂ atmosphere (1 atm.). The reaction mixture was filtered through Celite, the filtrate was concentrated. The crude product was purified using column chromatography using n-hexane as an eluent, obtaining yellow oil in 75% yield (0.61 g, 3.74 mmol).

¹H NMR (400 MHz, CDCl₃) δ ppm: 6.99 (d, J=7.7 Hz, 1H), 6.94 (d, J=7.7 Hz, 1H), 6.72 (t, J=7.7 Hz, 1H), 3.63 (s, 2H), 2.71-2.60 (m, 1H), 2.20 (s, 3H), 1.77-1.51 (m, 2H), 1.24 (d, J=7.0 Hz, 3H), 0.92 (t, J=7.3 Hz, 3H).

¹³C NMR (100 MHz, CDCl₃) δ ppm: 141.9, 131.2, 127.9, 124.0, 122.4, 118.4, 34.9, 29.6, 20.3, 18.2, 12.4.

### Alternative Embodiment R-3′

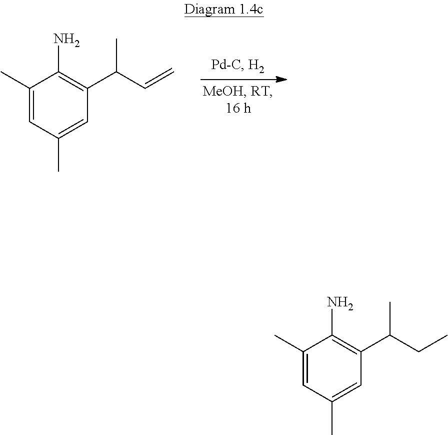

To a reaction vessel equipped with a stirring bar, containing a solution of aniline (20.0 g, 93.6 mmol, 1 equiv.) in MeOH (50 mL), Pd/C (1.0 g, 0.94 mmol, 0.01 equiv., 10% wt.) was added. The reaction flask was purged with hydrogen and the reaction was carried out at room temperature for 16 hours, in a H₂ atmosphere (1 atm.). The reaction mixture was filtered through Celite, the filtrate was concentrated. The crude product was purified by distillation under reduced pressure. Colourless oil was obtained in 88% yield (14.7 g, 83.1 mmol).

¹H NMR (400 MHz, CDCl₃) δ ppm: 6.81 (d, J=2.1 Hz, 1H), 6.78 (td, J=1.3, 0.6 Hz, 1H), 3.51 (s, 2H), 2.74-2.59 (m, 1H), 2.25 (s, 3H), 2.18 (s, 3H), 1.77-1.65 (m, 1H), 1.64-1.51 (m, 1H), 1.25 (d, J=6.8 Hz, 3H), 0.93 (t, J=7.4 Hz, 3H).

¹³C NMR (100 MHz, CDCl₃) δ ppm: 139.3, 131.4, 128.6, 127.4, 124.5, 122.6, 34.9, 29.8, 20.8, 20.3, 18.2, 12.5.

### Reaction R-1″

Synthesis of bis alkylated aniline with the general formula c″ is carried out in the R-1″ stage (Diagram 1). Aniline with general formula a′ and alkyl halide, preferably bromide, chloride or iodide, are used for this purpose. The transformation is preferably carried out in DMF or in a different organic solvent in the presence of a base, preferably triethylamine. The reaction is carried out at 0° C., up to room temperature. The product is separated from the reaction mixture through filtration, solvent distillation and column chromatography.

### Example Embodiment R-1″

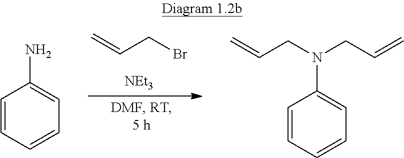

To a reaction vessel equipped with a stirring bar, containing a solution of aniline (6.9 g, 6.8 mL, 75 mmol, 1.5 equiv.) and triethylamine (3.2 g, 4.4 mL, 31.4 mmol, 0.63 equiv.) in DMF (25 mL) allyl bromide (6.2 g, 4.5 mL, 50 mmol, 1 equiv.) was added dropwise at 0° C. The contents of the flask were stirred at room temperature for 5 hours. The reaction was monitored using TLC. At the end of the reaction, water (50 mL) and EtOAc (50 mL), were added to the mixture, stirred for 5 minutes and the layers were separated. The water layer was extracted with EtOAc (2×25 mL). The combined organic layer was washed with water (2×30 mL) to remove traces of DMF, dried over anhydrous Na₂SO₄, filtered, the solvent was evaporated under reduced pressure. The crude product was purified using column chromatography, using n-hexane:EtOAc as the eluent (99:1), and light yellow oil was obtained in 35% yield (3.05 g, 17.6 mmol) [Synlett 2008, 19, 3011-3015].

### Reaction R-2″

An aza-Claisen rearrangement leading to an aniline with the general formula d′ is carried out in the R-2″ stage (Diagram 1). Aniline with general formula c″ and a Lewis acid, preferably boron trifluoride etherate, are used for this purpose. The reaction is preferably carried out in chlorobenzene, o-xylene or other organic solvent. The reaction is carried out at reflux. The product is separated from the reaction mixture through filtration, solvent distillation and column chromatography.

### Example Embodiment R-2″

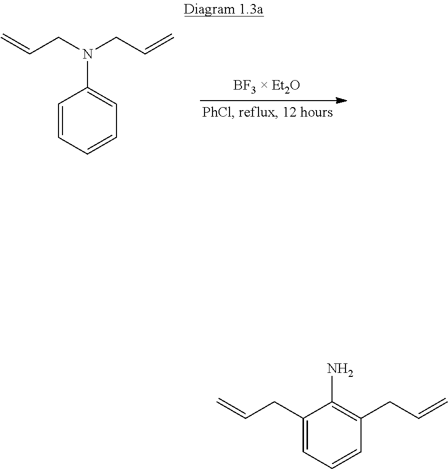

To a reaction vessel equipped with a stirring bar, containing a solution of aniline (3.12 g, 18 mmol, 1 equiv.) in chlorobenzene (20 mL), boron fluoride etherate (3.8 g, 3.3 mL, 27 mmol, 1.5 equiv.) was added slowly. The mixture was heated at reflux, under a reflux condenser, for 12 hours. Next, the mixture was cooled to room temperature and saturated solution of NaHCO₃ (20 mL) was added, the aqueous layer was extracted with EtOAc (2×25 mL), the combined organic fractions were washed with water (25 mL), brine (25 mL), and then dried over Na₂SO₄, filtered, the solvent was evaporated. The crude product was purified using column chromatography, using n-hexane as the eluent, and light yellow oil was obtained in 39% yield (1.2 g, 6.93 mmol) [Synlett 2008, 19, 3011-3015].

¹H NMR (400 MHz, CDCl₃) δ ppm: 7.00 (d, J=7.5 Hz, 2H), 6.75 (t, 1H), 6.04-5.92 (m, 2H), 5.18-5.10 (m, 4H), 3.75 (s, 1H), 3.38-3.32 (m, 4H).

¹³C NMR (100 MHz, CDCl₃) δ ppm: 143.3, 136.0, 128.6, 124.0, 118.4, 117.0, 36.7.

### Reaction R-3″

A hydrogenation reaction of unsaturated C═C bonds leading to an aniline with the general formula e″ is carried out in the R-3″ stage (Diagram 1). Aniline with general formula d″ and a catalyst, preferably Pd/C and gaseous hydrogen, are used for this purpose. The reaction is preferably carried out in methanol or other organic solvent. The reaction is carried out at room temperature. The product is separated from the reaction mixture through filtration, solvent distillation and distillation or column chromatography.

### Example Embodiment R-3″

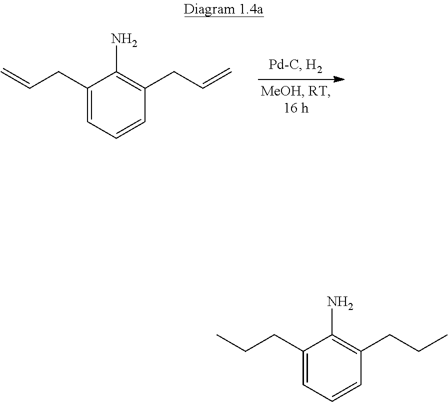

To a reaction vessel equipped with a stirring bar containing a solution of aniline (1.11 g, 6.4 mmol, 1 equiv.) in MeOH (50 mL) 10% wt. Pd/C (0.09 g, 0.08 mmol, 0.01 equiv., 10% wt.) was added. The reaction flask was purged with hydrogen and the reaction was carried out at room temperature for 16 hours, in a H₂ atmosphere (1 atm.). The reaction mixture was filtered through Celite, the filtrate was concentrated. The crude product was purified by distillation under reduced pressure.

Colourless oil was obtained in 74% yield (0.84 g, 4.7 mmol) [Eur. J. Med. Chem., 2019, 176, 162-174].

¹H NMR (400 MHz, CDCl₃) δ ppm: 6.97 (d, J=7.5 Hz, 2H), 6.73 (t, J=7.5 Hz, 1H), 3.64 (bs, 2H), 2.51 (t, J=7.5 Hz, 4H), 1.75-1.63 (m, 4H), 1.04 (t, J=7.4 Hz, 6H).

¹³C NMR (100 MHz, CDCl₃) δ ppm: 141.8, 127.2, 126.5, 118.0, 33.8, 21.9, 14.3.

### Example II

### Aldehyde Synthesis

The Diagram 2 presented below illustrates the synthesis of aldehydes used as building blocks for synthesis of CAAC ligands enabling ruthenium catalysts of olefin metathesis to be obtained (general formula from Ru-a to Ru-l, Diagram 10), comprising the subject of this invention.

Reaction R4 presented in Diagram 2 was carried out using commercially available substrates, according to procedures described in the literature, including changes developed by the authors. Unless written otherwise, the described reactions used commercially available solvents and no regard to the presence of oxygen and/or moisture was paid.

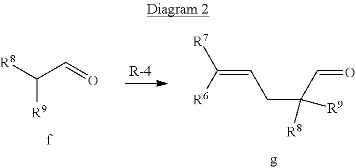

### Reaction R-4

During the R-4 stage (Diagram 2), the aldehyde alkylation reactions are carried out, which lead to the aldehyde with the general formula g under PTC conditions. An alkyl halide with the general formula f is used for the purpose, preferably chloride, bromide, iodide, base, preferably sodium hydroxide (NaOH) and a PTC catalyst, preferably tert-butylammonium bromide. The reaction is preferably carried out in a mixture of toluene (PhMe) and water. The reaction is carried out at 60° C. The product is separated from the reaction mixture through filtration, solvent distillation and distillation under reduced pressure.

### Example Embodiment R4

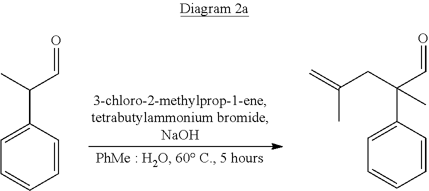

In a round-bottom flask with three necks and an installed reflux condenser, a drip funnel and a stirring bar, NaOH (17.90 g, 0.45 mol, 1.50 equiv.), (n-Bu)₄NBr (9.61 g, 29.8 mmol, 10% mol), 60 mL of distilled water and 400 mL of PhMe was placed. The mixture was heated to 60° C., and next a mixture of 2-phenylpropionic aldehyde (40.0 g, 40.0 mL, 0.30 mol, 1.00 equiv.) and 3-chloro-2-methylpropen (37.0 g, 40.0 mL, 0.40 mol, 1.33 equiv.) was added. The reaction was carried out at 60° C. for 5 hours. After cooling to room temperature, 30 mL of distilled water was added and the product was then extracted with PhMe. The organic layers were combined and dried over anh. Na₂SO₄, the precipitate was filtered and the solvent evaporated. The crude product was distilled under reduced pressure, colourless liquid was obtained in 70% yield (48.0 g, 0.21 mol).

¹H NMR (400 MHz, CDCl₃): δ ppm 9.55 (s, 1H), 7.41-7.35 (m, 2H), 7.32-7.27 (m, 3H), 4.84-4.79 (m, 1H), 4.65-4.61 (m, 1H), 2.75-2.63 (m, 2H), 1.47 (s, 3H), 1.41-1.39 (m, 3H).

¹³C NMR (101 MHz, CDC₃): δ ppm 202.1, 141.6, 139.9, 128.9, 127.5, 127.4, 115.6, 53.6, 44.3, 24.3, 18.7.

### Alternative Embodiment R-4

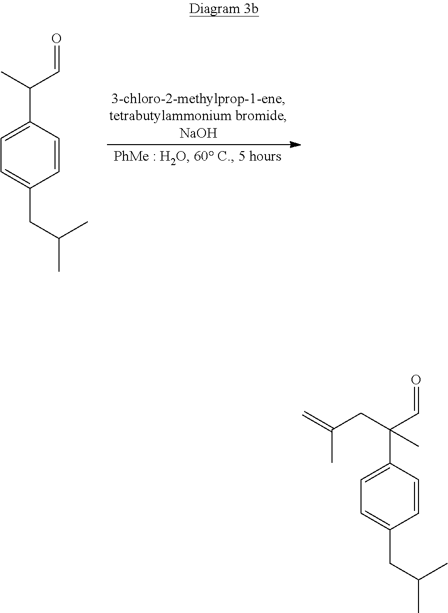 [SMILES: CC(C)Cc1ccc(C(C)C=O)cc1 ; C=C(C)CC(C)(C=O)c1ccc(CC(C)C)cc1]

In a round-bottom, three-neck flask fitted with a reflux condenser, a drip funnel and a stirring bar, NaOH (2.25 g, 38.0 mmol, 1.50 equiv.), (n-Bu)₄NBr (1.21 g, 3.7 mmol, 10 mol %), 5 mL of distilled water and 30 mL of PhMe were placed. The mixture was heated to 60° C., and the mixture of 2-(4-isobutylphenyl)propionic aldehyde (7.15 g, 37.6 mmol, 1.00 equiv.) and 3-chloro-2-methylpropene (4.67 g, 5.65 mL, 56 mmol, 1.4 equiv.) was added. The reaction was carried out at 60° C. for 5 hours. After cooling to room temperature, 30 mL of distilled water was added and the reaction mixture was then extracted with PhMe. The organic layers were combined and dried over anh. Na₂SO₄, the precipitate was filtered and the solvent evaporated. The crude product was distilled under reduced pressure and colourless liquid was obtained in 55% yield (5.0 g, 20.5 mmol).

¹H NMR (400 MHz, CDCl₃): δ ppm 9.52 (s, 1H), 7.22-7.11 (m, 4H), 4.83-4.77 (m, 1H), 4.66-4.59 (m, 1H), 2.75-2.58 (m, 2H), 2.46 (d, J=7.3 Hz, 2H), 1.92-1.77 (m, 1H), 1.45 (s, 3H), 1.41-1.36 (m, 3H), 0.89 (d, J=6.6 Hz, 6H).

¹³C NMR (101 MHz, CDCl₃): δ ppm 202.2, 129.6, 129.4, 129.2, 128.5, 128.4, 127.2, 53.3, 45.0, 44.3, 30.3, 24.2, 22.5, 18.6.

### Another Alternative Embodiment R-4

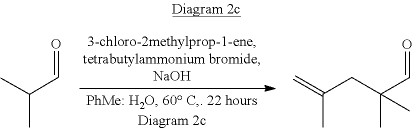

In a round-bottom, three-neck flask fitted with a reflux condenser, a drip funnel and a stirring bar, NaOH (4.16 g, 0.10 mol, 1.50 equiv.), (n-Bu)₄NBr (2.24 g, 6.93 mmol, 10 mol %), 14 mL of distilled water and 93 mL of toluene were placed. The mixture was heated to 60° C., and a mixture of 2-methylpropionic aldehyde (5.0 g, 6.3 mL, 69.3 mmol, 1.00 equiv.) and 3-chloro-2-methylpropene (8.6 g, 9.3 mL, 92.2 mmol, 1.33 equiv.) was added. The reaction was carried out at 60° C. for 22 hours. After cooling to room temperature, 90 mL of distilled water was added and the product was then extracted with toluene. The organic layers were combined and dried over anh. Na₂SO₄.

The precipitate was filtered. The product was used in the next step without further purification, as a solution in toluene.

### Example IIIa

### Synthesis of CAAC Ligand Precursors

The Diagram 3 below illustrates the synthesis of CAAC ligands enabling the ruthenium catalysts of olefin metathesis (general formulae of CAAC ligand precursors are shown in Diagram 3), comprising the subject of this invention, to be obtained.

The reactions R-5 and R-6 shown in Diagram 3 were carried out using commercially available compounds, according to procedures described in the literature, including changes developed by the authors. Unless written otherwise, the described reactions used commercially available solvents and no attention was paid to the presence of oxygen and/or moisture.

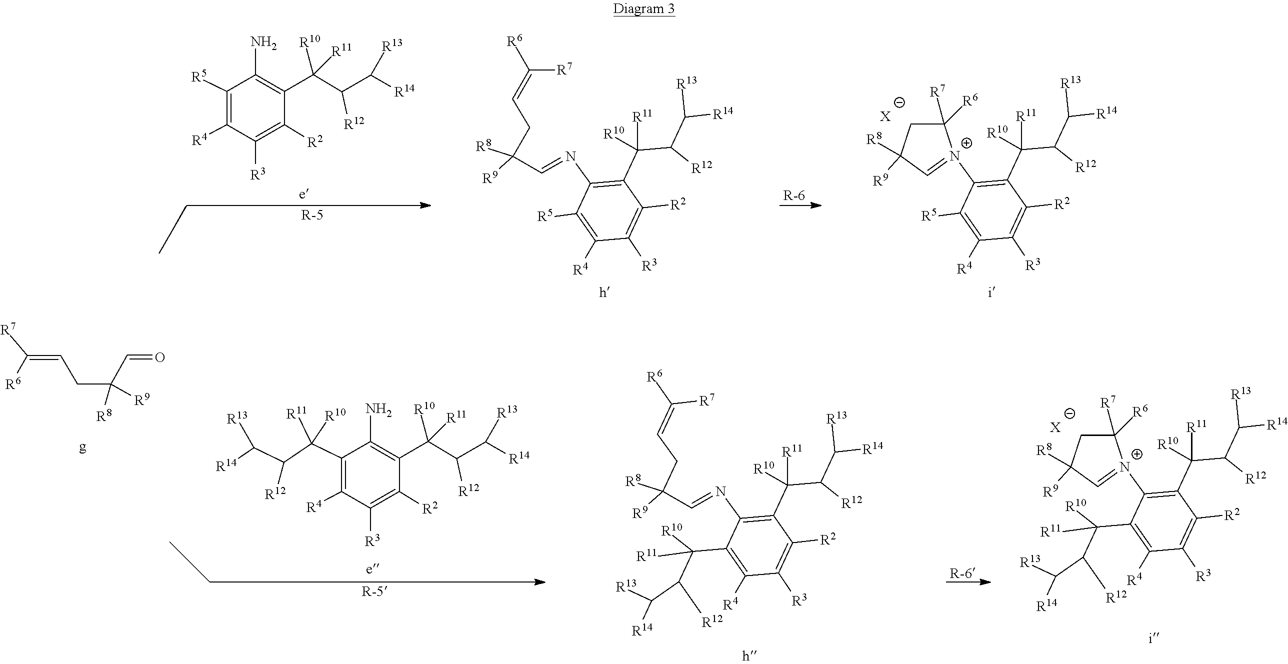

### Reaction R-5, R-6, R-5′ and R-6′

During the step R-5 or R-5′ (Diagram 3), the synthesis of h′ or h″ imine was carried out, using an aniline with the general formula of e′ or e″ and an aldehyde with the general formula g for the purpose, in the presence of trifluoroacetic acid, preferably of para-toluenesulphonic acid. The reaction is preferably carried out in PhMe or different organic solvent. The reaction mixture is stirred at reflux. The product is separated by filtration through neutral Al₂O₃ and solvent distillation. The product was used in the next step without further purification.

During the step R-6 or R-6′, the synthesis of the CAAC ligand precursor with the general formula i′ or i″ is carried out, using an imine with the general formula h′ or h″, obtained in R-5 or R-5′ step in the presence of trifluoroacetic acid, preferably 4N hydrochloric acid in dioxane. The reaction is carried out under protective argon atmosphere, in toluene, at 85° C. Next, the chloride ions are substituted with tetrafluoroborate ions and the crude product is precipitated from a MeOH/Et₂O mixture.

### Example Embodiment of the Invention

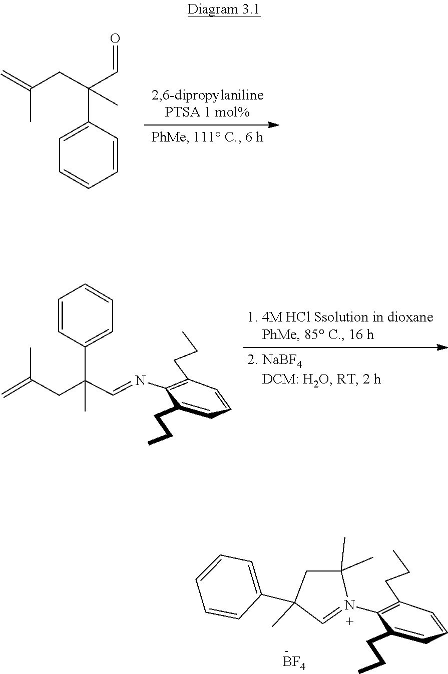

In a round-bottom flask equipped with a stirring bar, 2,4-dimethyl-2-phenylpent-4-enal (0.74 g, 3.9 mmol, 1.00 equiv.), 2,6-dipropylaniline (0.7 g, 3.9 mmol, 1.00 equiv.) and PTSA (6.8 mg, 0.04 mmol, 1 mol %) were placed and dissolved in PhMe (at a concentration C=0.30 M). The reaction was carried out at reflux, up to the total conversion of substrates (collecting water in a Dean-Stark apparatus). The solvent was evaporated under reduced pressure, the crude reaction mixture was filtered through neutral aluminium oxide (Al₂O₃, Broockman Grade 1), dried in vacuo, obtaining imine which was used in the next step without further purification, in 91% yield (1.25 g, 3.6 mmol).

In a round-bottom flask, under a protective argon atmosphere, imine from the previous step, 4 M HCl (2.23 g, 2.27 mL, 9.06 mmol, 2.5 equiv.), solution in dioxane and PhMe (at a concentration C=0.50 M) were placed. The reaction was carried out for 16 hours at 85° C. The solvent was evaporated under reduced pressure. The crude product was dissolved in water/methylene chloride mixture, NaBF₄ (0.80 g, 7.25 mmol) was added and ion exchange was carried out for 2 hours. The organic fraction was collected, washed with water and dried over anh. sodium sulphate. The product was precipitated from a MeOH:Et₂O mixture, giving colourless crystals in 75% yield (1.19 g, 2.7 mmol).

¹H NMR (400 MHz, CDCl₃): δ ppm δ 9.50 (s, 1H), 7.54-7.49 (m, 2H), 7.47-7.38 (m, 3H), 7.37-7.31 (m, 1H), 7.30-7.26 (m, 1H), 7.24-7.20 (m, 1H), 3.22 (d, J=14.1 Hz, 1H), 2.67 (d, J=14.1 Hz, 1H), 2.59-2.38 (m, 2H), 2.36-2.23 (m, 1H), 2.11-1.99 (m, 1H), 1.92 (s, 3H), 1.80-1.41 (m, 7H), 1.33 (s, 3H), 0.97 (t, J=7.3 Hz, 3H), 0.71 (t, J=7.3 Hz, 3H).

¹³C NMR (101 MHz, CDC₃): δ ppm 189.6, 140.4, 138.6, 138.0, 131.2, 131.0, 130.1, 128.6, 128.4, 128.2, 125.9, 84.0, 55.6, 48.3, 34.3, 34.1, 29.2, 27.1, 26.9, 25.0, 24.1, 14.4, 14.1.

### Alternative Embodiment of the Invention

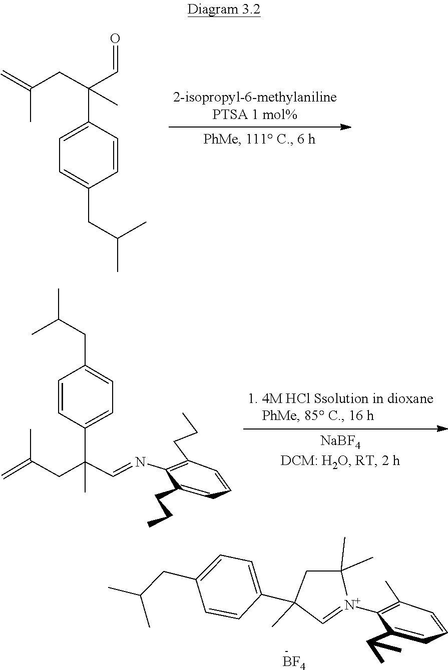

In a round-bottom flask equipped with a stirring bar 2-(4-isobutylphenyl)-2,4-dimethylopent-4-enal (3.40 g, 8.4 mmol, 1.00 equiv.), aniline (1.25 g, 8.4 mmol, 1.00 equiv.) and PTSA (14 mg, 0.08 mmol, 1 mol %) were placed and dissolved in PhMe (at a concentration of C=0.30 M). The reaction was carried out at reflux, up to the total conversion of substrates (collecting water in a Dean-Stark apparatus). The solvent was evaporated under reduced pressure, the crude reaction mixture was filtered through neutral aluminium oxide (Al₂O₃, Broockman Grade 1), dried under in vacuo, obtaining imine which was used in the next step without further purification, in 65% yield (2.03 g, 5.4 mmol).

In a round-bottom flask, under a protective argon atmosphere, imine from the previous step, 4 M HCl (2.43 g, 3.33 mL, 13.3 mmol, 2.5 equiv.), solution in dioxane and PhMe (at a concentration C=0.50 M) were placed. The reaction was carried out for 16 hours at 85° C. The solvent was evaporated under reduced pressure. The crude product was dissolved in a water/methylene chloride mixture and NaBF₄ was added (1.17 g, 10.6 mmol, 2.0 equiv.) and ion exchange was carried out for 2 hours. The organic fraction was collected, washed with water and dried over anh. sodium sulphate. The product was precipitated from a MeOH:Et₂O mixture, giving colourless crystals in 61% yield (1.51 g, 3.3 mmol).

¹H NMR (400 MHz, CDCl₃): δ ppm 9.52 (s, 0.67×1H), 9.47 (s, 0.33×1H), 7.47-7.30 (m, 4H), 7.25-7.15 (m, 3H), 3.25-3.12 (m, 1H), 2.74-2.61 (m, 2H), 2.51-2.42 (m, 2H), 2.39-2.23 (m, 2H), 2.07 (s, 2H), 1.96 (s, 1H), 1.92-1.79 (m, 3H), 1.61 (s, 1H), 1.55 (s, 2H), 1.41-1.33 (m, 5H), 1.21-1.13 (m, 3H), 0.97 (d, J=6.7 Hz, 1H), 0.92-0.85 (m, 6H).

¹³C NMR (101 MHz, CDCl₃): δ ppm 189.4, 140.7, 139.7, 139.2, 131.3, 130.6, 129.8, 128.3, 127.8, 127.6, 125.6, 83.9, 55.4, 48.0, 28.9, 26.9, 26.6, 24.9, 24.6, 15.4, 14.6.

### Alternative Embodiment of the Invention

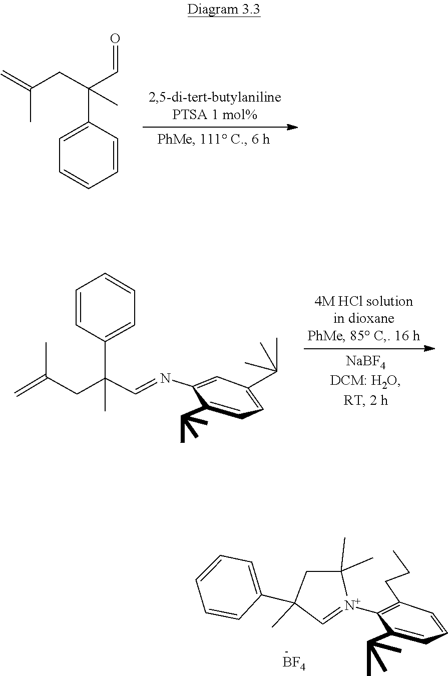

In a round-bottom flask equipped with a stirring bar, 2,4-dimethyl-2-phenylpent-4-enal (1.00 g, 5.3 mmol, 1.00 equiv.), aniline (1.10 g, 5.3 mmol, 1.00 equiv.) and PTSA (10 mg, 0.05 mmol, 1 mol %) were placed and dissolved in PhMe (C=0.30 M). The reaction was carried out at reflux, up to the total conversion of substrates (collecting water in a Dean-Stark apparatus). The solvent was evaporated under reduced pressure, the crude reaction mixture was filtered through neutral aluminium oxide (Al₂O₃, Broockman Grade 1), dried in vacuo, obtaining imine which was used in the next step without further purification, in 98% yield (1.95 g, 5.2 mmol).

In a round-bottom flask, under a protective argon atmosphere, imine from the previous step, 4 M HCl (6.90 g, 6.57 mL, 26.3 mmol, 2.5 equiv.), solution in dioxane and PhMe (C=0.50 M) were placed. The reaction was carried out for 16 hours at 85° C. The solvent was evaporated under reduced pressure. The crude product was dissolved in a water/methylene chloride mixture and NaBF₄ was added (2.31 g, 21.0 mmol, 2.0 equiv.) and ion exchange was carried out for 2 hours. The organic fraction was collected, washed with water and dried over sodium sulphate. The product was precipitated from a MeOH:Et₂O mixture, giving colourless crystals in 72% yield (1.73 g, 3.7 mmol).

### Another Alternative Embodiment of the Invention

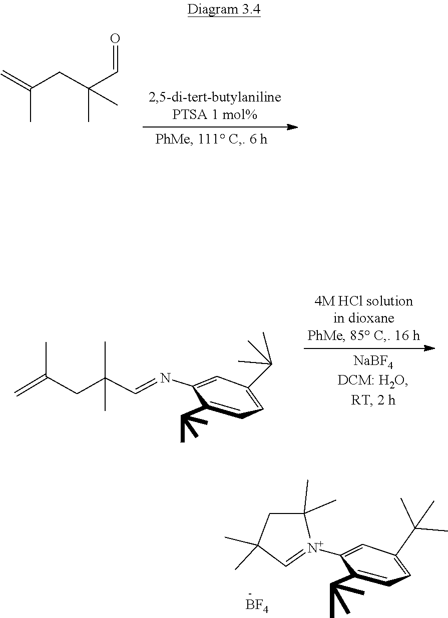

In a round-bottom flask equipped with a stirring bar, 2,2,4-trimethylpent-4-enal (1.01 g, 8.0 mmol, 1.00 equiv.), aniline (2.0 g, 9.6 mmol, 1.2 equiv.) and PTSA (15 mg, 0.05 mmol, 1 mol %) were placed and dissolved in toluene (C=0.30 M). The reaction was carried out at reflux, up to the total conversion of substrates (collecting water in a Dean-Stark apparatus). The solvent was evaporated under reduced pressure, the crude reaction mixture was filtered through neutral aluminium oxide (Al₂O₃, Broockman Grade I), dried in vacuo, obtaining imine which was used in the next step without further purification, in 99% yield (2.5 g, 8.0 mmol).

In a round-bottom flask, under a protective argon atmosphere, imine from the previous step, 4 M HCl (5.13 g, 4.98 mL, 19.9 mmol, 2.5 equiv.), solution in dioxane and toluene (C=0.50 M) were placed. The reaction was carried out for 16 hours at 85° C. The solvent was evaporated under reduced pressure. The crude product was dissolved in a water/methylene chloride mixture and NaBF₄ was added (1.75 g, 15.9 mmol, 2.0 equiv.) and ion exchange was carried out for 2 hours. The organic fraction was collected, washed with water and dried over anh. sodium sulphate. The product was precipitated from a MeOH:Et₂O mixture, giving colourless crystals in 25% yield (0.8 g, 2.0 mmol).

¹H NMR (400 MHz, CDCl₃): δ ppm δ 8.85 (s, 1H), 7.67 (d, J=8.6 Hz, 1H), 7.58 (dd, J=2.1, 8.7 Hz, 1H), 7.00 (d, J=2.1 Hz, 1H), 2.47 (d, J=13.8 Hz, 1H), 2.31 (d, J=13.8 Hz, 1H), 1.72 (s, 31H), 1.64 (s, 3H), 1.53 (s, 3H), 1.48 (s, 3H), 1.37 (s, 9H), 1.32 (s, 9H).

¹³C NMR (101 MHz, CDCl₃): δ ppm 189.8, 150.7, 141.8, 131.9, 131.2, 128.8, 124.2, 83.0, 49.1, 47.7, 37.6, 34.6, 33.8, 30.9, 30.9, 27.0, 25.6, 25.6.

Using the reaction R-6, R-6′ presented above, the following compounds with the general formula of CAAC ligand precursors were obtained.

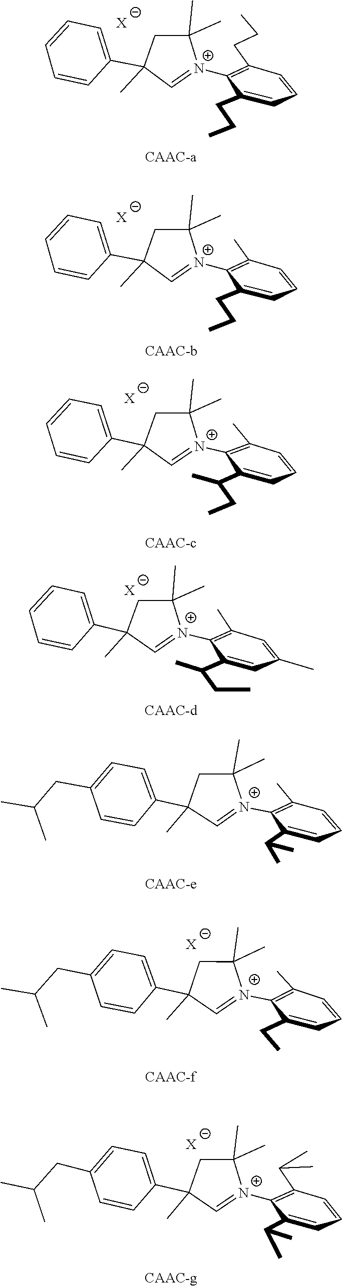

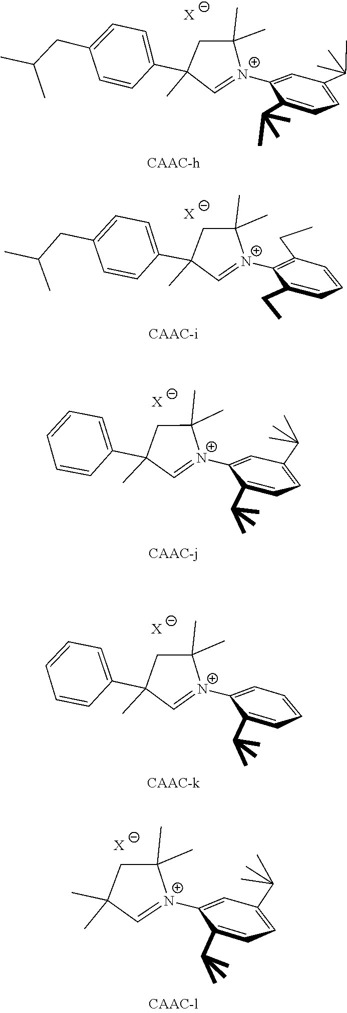

### Example IIIb

### Synthesis of NHC Ligand Precursors

The Diagram below illustrates the synthesis of NHC ligands enabling synthesis of the olefin metathesis ruthenium catalysts (general formula I to IV, synthesis I-III), comprising the subject of this invention, to be obtained.

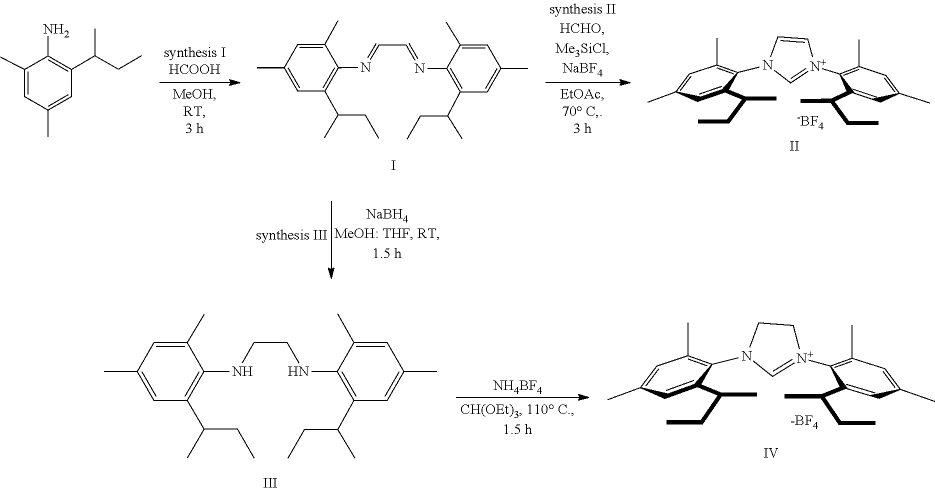

### Synthesis I

To a reaction vessel equipped with a stirring bar, containing a solution of 2-(sec-butyl)-4,6-dimethylaniline (8.03 g, 45.3 mmol, 2 equiv.) in methanol (MeOH) (9 mL) glyoxal solution (40% aqueous solution) (3.45 g, 2.73 mL, 23.8 mmol, 1.05 equiv.) was added dropwise, the reaction was carried out at room temperature for 20 hours. The crude product was filtered, washed with methanol and dried under reduced pressure. A yellow precipitate was obtained in 63% yield (5.43 g, 14.4 mmol).

### Synthesis II

Under a protective argon atmosphere, ethyl acetate (64 mL) was added to the reaction vessel equipped with a stirring bar and heated to 70° C. Next, imine I (2.71 g, 7.19 mmol, 1 equiv.) and paraformaldehyde (225 mg, 7.26 mmol, 1.01 equiv.) were added, followed by the addition of chlorotrimethylosilane (9.21 mL, 7.19 mmol, 1.00 equiv.), dissolved in ethyl acetate (0.7 mL). The reaction was carried out at 70° C. for 2 hours. The mixture was cooled and the precipitate filtered, washed with ethyl acetate and tert-butyl-methyl ether. The precipitate was dissolved in dichloromethane (20 mL) and a solution of potassium tetrafluoroborate (2.71 g, 7.2 mmol, 1.0 equiv.) in water (15 mL) was added, and the mixture was stirred for 2 hours. The mixture was extracted using methylene chloride (3×30 mL). The organic fractions were collected, dried over anh. magnesium sulphate and filtered through neutral Celite. The solvent was evaporated under reduced pressure, the crude product was recrystallised from a methanol/diethyl ether mixture. A colourless precipitate was obtained in 66% yield (2.27 g, 4.76 mmol).

### Synthesis III

Under a protective argon atmosphere, to a reaction vessel equipped with a stirring bar, containing imine I (2.64 g, 7.01 mmol, 1 equiv.) in a methanol—tetrahydrofuran mixture (36 mL+53 mL), sodium borohydride (2.71 g, 70.1 mmol, 10.0 equiv.) was added in portions keeping a temperature below 0° C., and next the reaction was carried out at room temperature for 1.5 hours. Ammonium chloride was added to the reaction mixture. The reaction mixture was extracted with diethyl ether (3×50 mL) and washed with distilled water (20 mL). The organic fractions were collected, dried over anh. magnesium sulphate, filtered through neutral Celite and the solvent was evaporated under reduced pressure. A yellow oil was obtained in 99% yield (3.13 g, 6.94 mmol).

### Synthesis IV

Under a protective argon atmosphere, to a reaction vessel equipped with a stirring bar, amine III (2.53 g, 6.65 mmol, 1 equiv.), triethyl orthoformate (2.68 mL, 16.6 mmol, 2.5 equiv.) and ammonium tetrafluoroborate (767 mg, 7.32 mmol, 1.1 equiv.) were added. The reaction was carried out at 110° C. for 1.5 hours. The mixture was cooled and extracted with methylene chloride (3×50 mL). The collected organic fractions were dried over magnesium sulphate, filtered through neutral Celite and the solvent was evaporated under reduced pressure. The crude product was crystallised from a chloroform/diethyl ether mixture. Colourless crystals were obtained in 49% yield (1.56 g, 3.27 mmol).

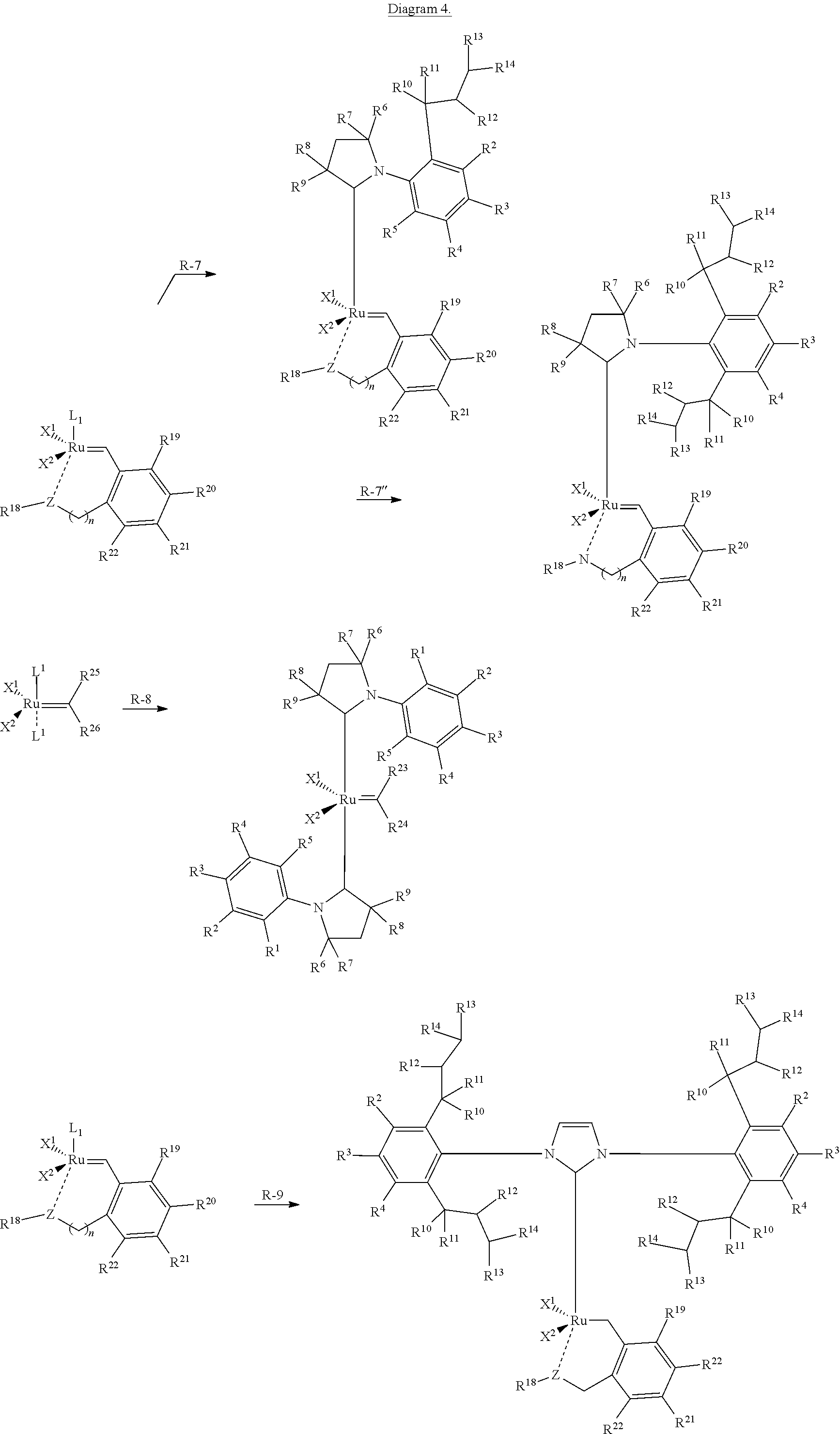

### Example IV

### Synthesis of Ruthenium Complexes Ru-a-Ru-o Using CAAC Ligands

### Example Embodiment of the Invention

### Method A

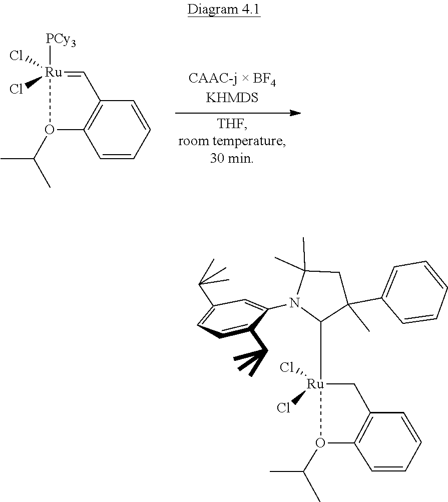

Under a protective argon atmosphere, in a preheated Schlenk vessel, the CAAC-j×BF₄ (339 mg, 0.73 mmol, 2.20 equiv.) and THF (C_{cA}c=0.03 M) were placed, followed by the addition of KHMDS (139 mg, 0.83 mmol, 2.50 equiv.) and stirred for 30 minutes. Next, the 1^{st} generation Hoveyda-Grubbs complex was added (200 mg, 0.33 mmol, 1 equiv.) and stirred until full conversion. The crude mixture was filtered through SiO₂ with toluene as an eluent. The green fraction was collected and the solvents were evaporated under reduced pressure. The complex was purified by column chromatography (SiO₂, 0→25% EtOAc/n-hexane), giving the final product as a green crystalline solid in 76% yield (175 mg, 0.25 mmol).

### Alternative Embodiment of the Invention

### Method B

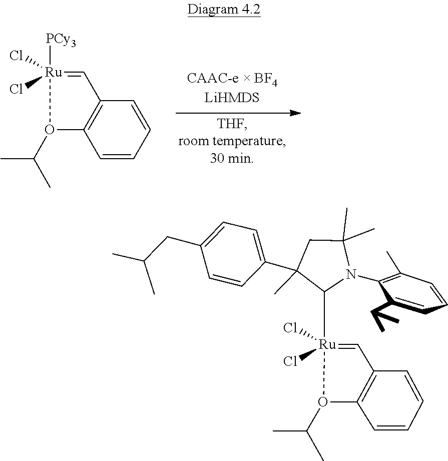

Under a protective argon atmosphere, in a preheated Schlenk vessel, the CAAC-e×BF₄ (255 mg, 0.55 mmol, 2.20 equiv.), 1^{st} generation Hoveyda-Grubbs complex (150 mg, 0.25 mmol, 1.00 equiv.) and THF were placed (C_{CAAC} concentration=0.1 M), and the mixture was stirred for 1 minute. Next, LiHMDS (92 mg, 0.55 mg, 2.20 equiv.) was added and the mixture was stirred until full conversion. The crude mixture was filtered through neutral aluminium oxide (Al₂O₃, Broockman Grade I) with Et₂O as the eluent. The green fraction was collected and evaporated under reduced pressure. Next, a small quantity of n-pentane was added and the mixture was placed in an ultrasound bath. The product was filtered and washed with cold n-pentane. After drying in vacuo, green crystalline solid was obtained in 45% yield (75 mg, 0.11 mmol).

### Alternative Embodiment of the Invention

### Method C

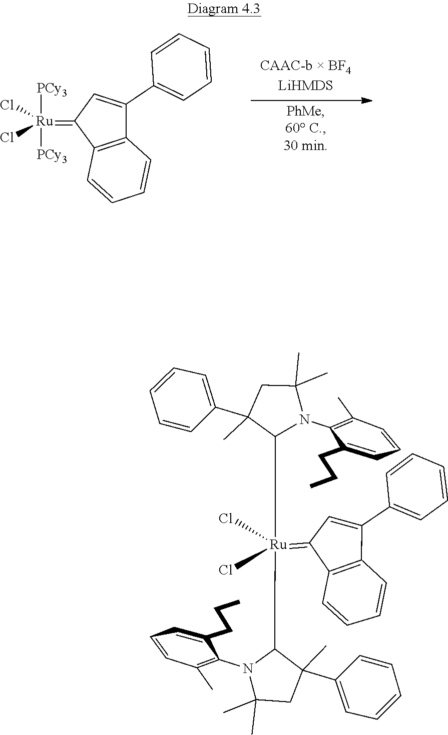

Under a protective argon atmosphere, in a preheated Schlenk vessel, the CAAC-b×BF₄ (132 mg, 0.33 mmol, 3.0 equiv.), the 1st generation indenylidene complex (100 mg, 0.11 mmol, 1.0 equiv.) and toluene (C_{c}Ac=0.16 M), were stirred for 1 minute. Next, LiHMDS (54 mg, 0.33 mg, 3.0 equiv.) was added, the mixture was placed in an oil bath heated to 60° C. and stirred until full conversion. Once the vessel was cooled, the crude mixture was filtered through Celite and concentrated. Next, a small quantity of n-pentane was added and the mixture was placed in an ultrasound bath. The product was filtered and washed with cold n-pentane. After drying in vacuo, red crystalline solid was obtained in 14% yield (15 mg, 0.015 mmol).

### Alternative Embodiment of the Invention

### Method D

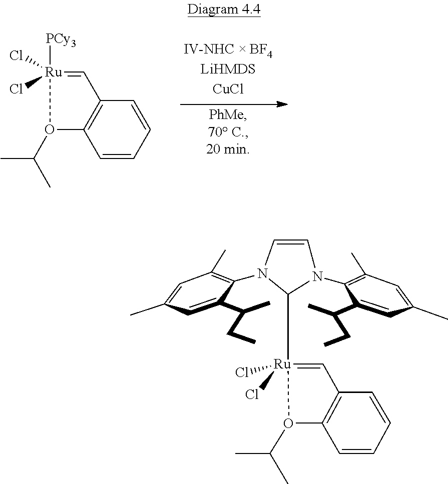

Under a protective argon atmosphere, in a preheated Schlenk vessel, IV-NHC×BF₄ (59.5 mg, 125 mol, 1.50 equiv.), 1st generation Hoveyda-Grubbs complex (50.0 mg, 83.2 mol, 1.00 equiv.) and anhydrous THF (C_{NHC}=0.07 M) were placed and stirred for 1 minute. Next, a solution of LiHMDS in anhydrous toluene (242 μl, 517 mM in toluene, 125 μmol, 1.50 equiv.) was added and stirred until full conversion (10 min) at 70° C. Copper(I) chloride (25.0 mg, 250 μmol, 3.00 equiv.) was added next and stirred for 10 min at 70° C. The crude mixture was filtered through neutral aluminium oxide (Al₂O₃, Broockman Grade 1) with diethyl ether as the eluent. The green fraction was collected and the solvents were evaporated under reduced pressure. After drying in vacuo, green crystalline solid was obtained in 76% yield (45.0 mg, 63.5 μmol).

Using methods A-D presented in example IV, a range of complexes from Ru-a to Ru-n was obtained, the structures of which are presented below.

All complexes from the table below were characterised using magnetic nuclear resonance spectroscopy. Table 1 contains the shifts of benzylidene/indenylidene protons for each of the complexes in the ¹H NMR spectrum, in the given solvent.

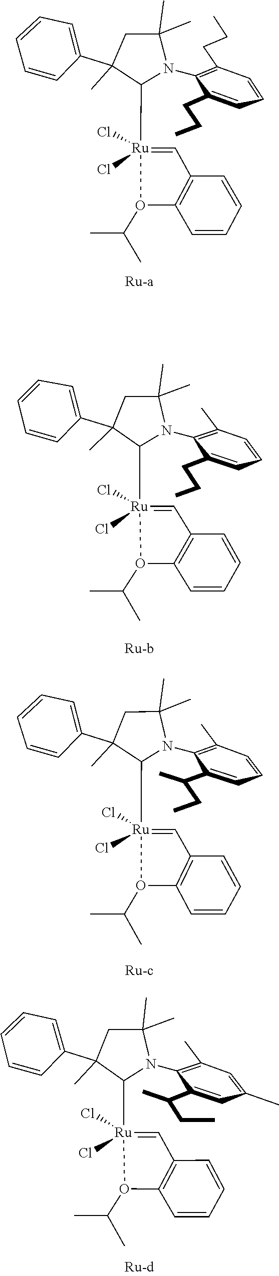

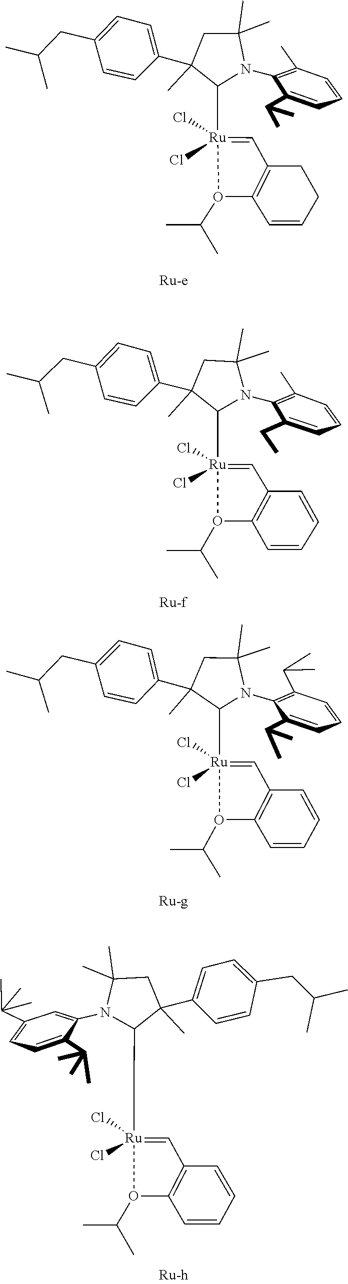

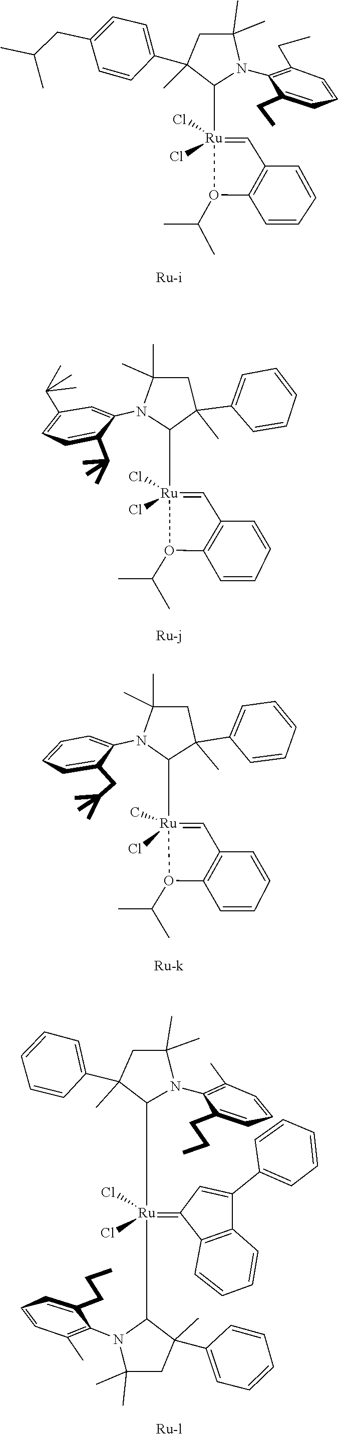

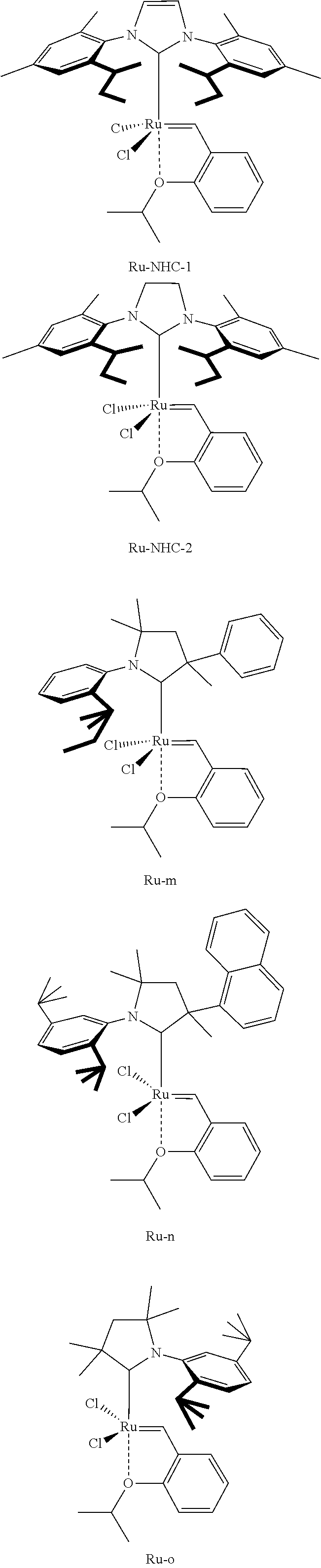

TABLE 1
A summary of the obtained ruthenium complex structures according to the general
procedure of Example IV and shifts of their benzylidene/indenylidene proton in the ¹H NMR
spectrum.

| | | | | | ¹H NMR δ |
|---|---|---|---|---|---|
| | | | | Molar | (ppm) |
| | Compound | | Stoichiometric | mass | Ru = CH |
| No. | no. | Structure | formula | [g/mol] | (solvent) |
| 1 | Ru-a | 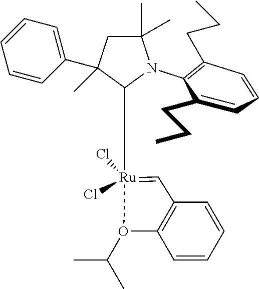 | C₃₅H₄₅Cl₂NORu | 667.72 | δ 17.72; 16.41 (CDCl₃) |
| 2 | Ru-b | 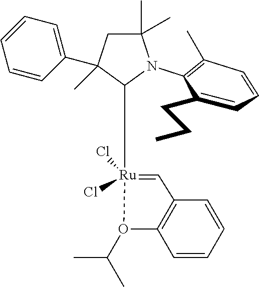 | C₃₃H₄₁Cl₂NORu | 639.67 | δ 17.77; 16.34 (CDCl₃) |
| 3 | Ru-c | 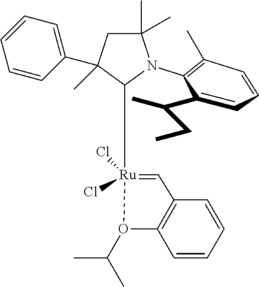 | C₃₄H₄₃Cl₂NORu | 653.69 | δ 16.31; 16.27; 16.22 (CD₂Cl₂) |
| 4 | Ru-d | 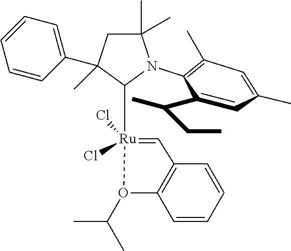 | C₃₅H₄₅Cl₂NORu | 667.72 | δ 16.23; 16.21 (CD₂Cl₂) |
| 5 | Ru-e | 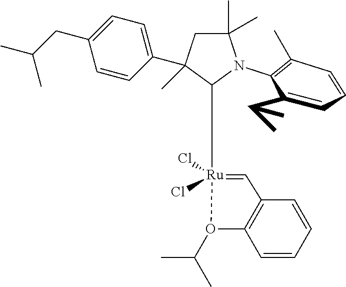 | C₃₇H₄₉Cl₂NORu | 695.22 | δ 16.37; 16.35 (CDCl₃) |
| 6 | Ru-f | 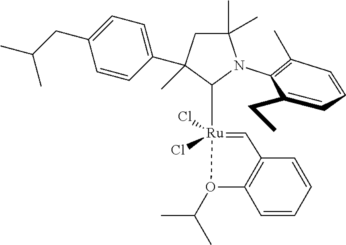 | C₃₆H₄₇Cl₂NORu | 681.21 | δ 17.73, 16.36, 16.33 (CDCl₃) |
| 7 | Ru-g | 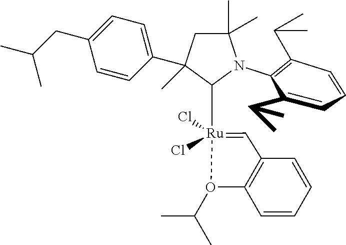 | C₃₉H₅₃Cl₂NORu | 723.83 | δ 16.56 (CDCl₃) |
| 8 | Ru-h | 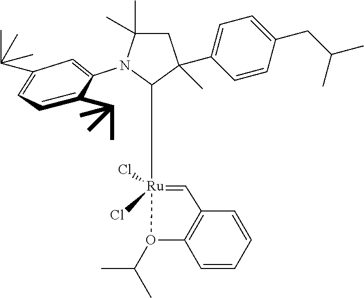 | C₄₁H₅₇Cl₂NORu | 751.29 | δ 17.84; 16.79 (CDCl₃) |
| 9 | Ru-i | 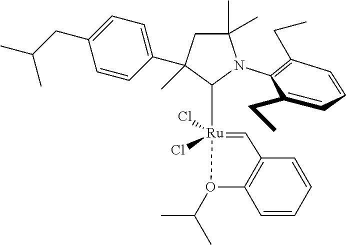 | C₃₇ H₄₉Cl₂NORu | 695.22 | δ 17.73, 16.44 (CDCl₃) |
| 10 | Ru-j | 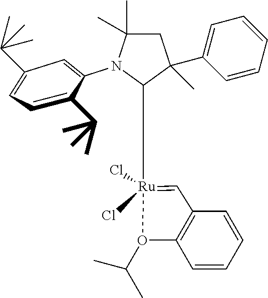 | C₃₇H₄₉Cl₂NORu | 695.78 | δ 17.85 (CD₂Cl₂) |
| 11 | Ru-k | 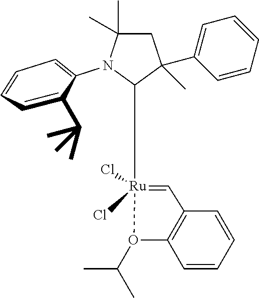 | C₃₃H₄₁Cl₂NORu | 639.16 | δ 18.00 (CDCl₃) |
| 12 | Ru-I | 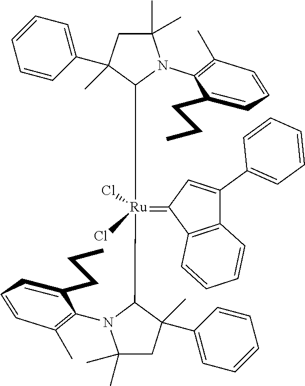 | C₆₁H₆₈Cl₂N₂Ru | 1001.20 | δ 9.66-9.48 (C₆D₆) |
| 13 | Ru-m | 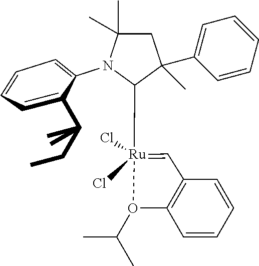 | C₃₄H₄₃Cl₂NORu | 653.18 | δ 18.00; 16.82 (CDCl₃) |
| 14 | Ru-n | 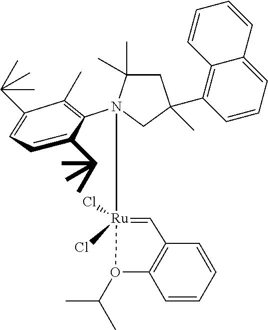 | C₄₁H₅₁Cl₂NORu | 745.24 | δ 18.34; 18.18 (CDCl₃) |
| 15 | Ru-o | 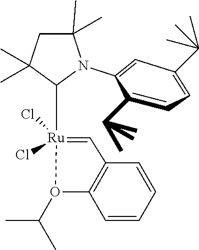 | C₃₂H₄₇Cl₂NORu | 633.21 | δ 16.59 (CDCl₃) |
| 16 | Ru- NHC-1 | 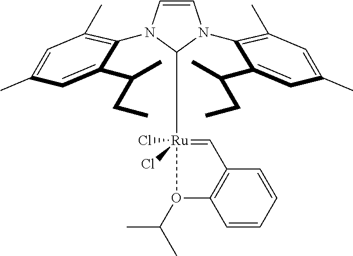 | C₃₇H₅₀Cl₂N₂ORu | 708.22 | δ 16.71-16.68, 16.66, 16.64 (CD₂Cl₂) |
| 17 | Ru- NHC-2 | 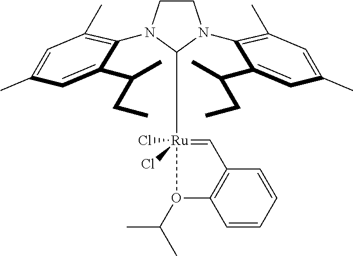 | C₃₇H₅₀Cl₂N₂ORu | 710.79 | δ 16.52-16.48, 16.47-16.46 (CD₂Cl₂) |

### Example V

### Test of the Activity of Complexes in the Ethenolysis Reaction of Methyl Oleate

A Schlenk flask equipped with a magnetic stirring element (previously weighed with a stopper on the balance) [photo A] was connected to a Schlenk line. Next, a sintered-glass filtration funnel was mounted in the top of the Schlenk tube [photo B] and filled with Al₂O₃[photo C & C′]. The filtration funnel was evacuated for 30 minutes and filled with argon. Methyl oleate (95% pure) or FAME (containing 73% of methyl oleate and 19.7% methyl linoleate) (15 mmol) was taken from ampule using syringe and transferred to the filtration funnel [photo D & D′] to be filtered through the pad of Al₂O₃ using pressurized argon. Next, the filtered methyl oleate or FAME [photo E] was degassed under vacuum (p~1×10⁻² mbar) for 10 min [photo F] and the Schlenk flask with substrate was weighed again (and the amount of methyl oleate or FAME was calculated from the mass difference). In a separate Schlenk flask under argon atmosphere, a pre-weighted sample of catalyst (Ru5-Ru10) was dissolved in anh. PhMe (c=1 mg/mL). The appropriate amount of the catalyst solution (corresponding to 0.5-15 ppm of Ru) was added under argon to a Schlenk flask containing filtered methyl oleate or FAME using a Hamilton syringe (the amount of dissolved catalyst was precisely calculated with reference to the mass of the substrate in the Schlenk flask) and mixed properly by shaking the Schlenk flask for 5 seconds. The resulting mixture was immediately sucked into a steel autoclave [photo G] containing a glass reacting chamber equipped with a magnetic stirring element through a Teflon tube [photo H & 1] using vacuum, Next, the autoclave was filled with ethylene (10 bar) and the reaction mixture was stirred at 40° C. for 6 hours. After that time, pressure was normalized and the autoclave was disassembled, A solution of SnatchCat^{1,2} in DCM (0.5 mL) was added to the reaction mixture, which was stirred for 5 mS. Next, a sample of the reaction mixture was taken, and subjected to GC analysis. Each sample was measured 4 times and results are the average of 4 measurements where the where the deviations between the averaged result and each of the measurements are

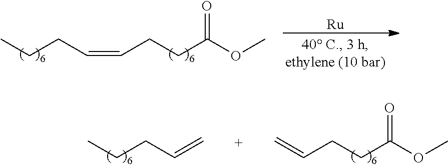

The results of a model reaction are presented in Table 2.

TABLE 2

| | | | Ethenolysis results | | |
|---|---|---|---|---|---|
| | Compound | | | | Selectivity |
| No. | no. | Structure | Loading | TON | [%] |
| 1 | Ru-a | 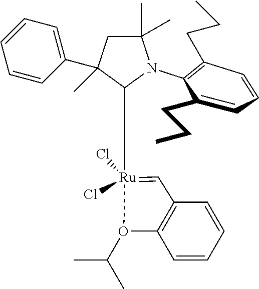 | 10 ppm | 62,660 | 87 |
| 2 | Ru-b | 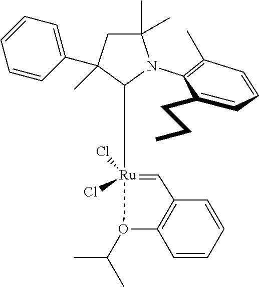 | 10 ppm | 62,660 | 87 |
| 3 | Ru-c | 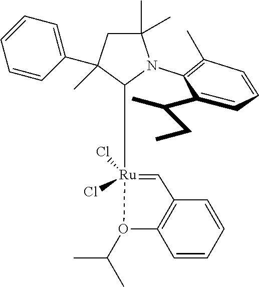 | 3 ppm 10 ppm | 155,166 55.388 | 94 93 |
| 4 | Ru-e | 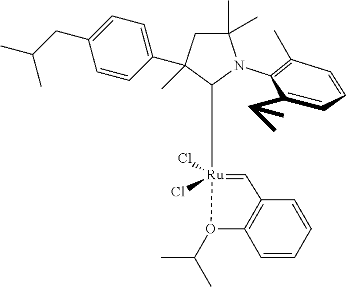 | 3 ppm 10 ppm | 186,264 72.344 | 94 90 |
| 5 | Ru-h | 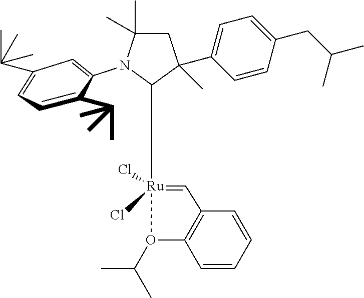 | 3 ppm 10 ppm | 183,020 67,712 | 88 83 |
| 6 | Ru-j | 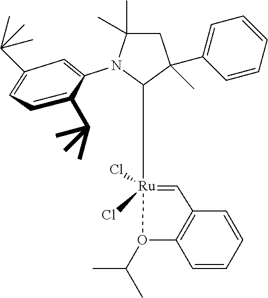 | 3 ppm 3 ppm 10 ppm 10 ppm 10 ppm | 207,740 211,644 70,306 71,525 70,483 | 93 88 82 84 83 |
| 7 | Ru-5 | 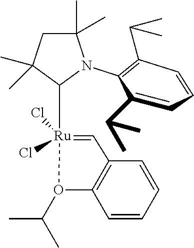 | 100 ppm 50 ppm | 5,600 11,400 | 90 93 |
| 8 | Ru-10 | 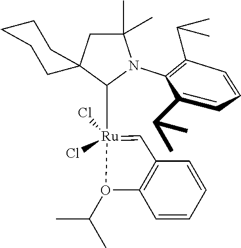 | 100 ppm 3 ppm | 4,200 43,000 | 94 97 |
| 9 | Ru-2 | 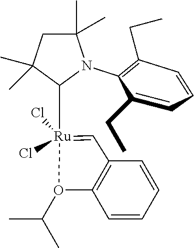 | 100 35 10 | 5,300 16,000 35,000 | 73 75 83 |

### Example VI

### Test of Complex Solubility in Nonpolar Solvents

In a round-bottom flask equipped with a stirring bar, the catalyst (Ru-a, Ru-b, Ru-c, Ru-d, Ru-h, Ru-15, Ru-16 and Hov-II) was placed, in the amount of 5 mg of each. n-hexane was added to each of the flasks in portions, at room temperature, and the mixture was stirred using a magnetic stirrer. The Ru-a dissolved in 6 mL, and the Ru-h complex in 4 mL of the solvent. In the case of the Ru-15 complex, almost complete solubility was observed after adding 20 mL of n-hexane. The Hov-II complex did not dissolve even in 20 mL of the solvent, even after 4 hours of intensive stirring.

TABLE 3
Solubility tests of ruthenium complexes of the CAAC type, Hoveyda-Grubbs type Ru16
(benchmark), Ru-a, Ru-b, Ru-c, Ru-d in n-hexane at room temperature

| | Ru-16 | Ru-c | Ru-d | Ru-b | Ru-a |
|---|---|---|---|---|---|
| Ru complex | 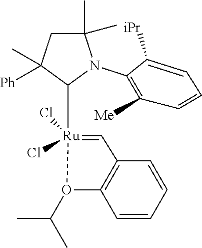 | 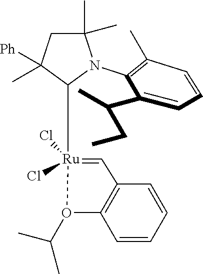 | 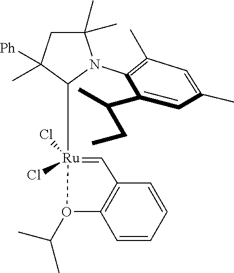 | 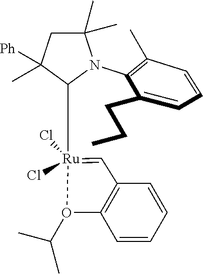 | 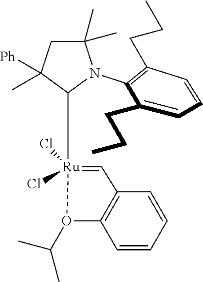 |
| Solubility | 0.039 | 0.398 | 0.414 | 0.638 | 0.790 |
| [mg mL⁻¹] | | | | | |

### Example VII

### Tests of Complex Activity in the Self-Cross Metathesis Reaction of 1-Dodecene.

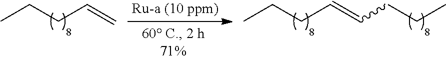

Before the reaction, 1-dodecene was distilled over activated, neutral aluminium oxide (Al₂O₃) under reduced pressure. Schlenk flask equipped with a stirring bar was dried under reduced pressure. 1,3,5-trimethoxybenzene (ca. 5 g) was added to the Schlenk flask and Ru-a (1.78 mg, 2.67 mol, 0.001 mol %) was placed in a dried vial. Freshly distilled and stored with activated aluminium oxide 1-dodecene (47.0 g, 267 mmol, 1 equiv., 95.6% purity) was transferred using syringe glass filter (1-2 μm) to previously prepared Schlenk flask and then reaction mixture was degassed in vacuo. Vial with solid catalyst was dipped in reaction mixture and stirred at 60° C. under argon atmosphere for 2 h. Conversion and selectivity were estimated using GC.

### Example VIII

### Individual Reactions Comprising the Synthesis Method of Novel Precursors of the CAAC Ligand

### Method A

In a round-bottom flask equipped with a magnetic stirring element, 2,4-dimethyl-2-phenylpent-4-enal (4b) (1.00 equiv.), aniline (1.00 equiv.) and PTSA (1 mol %) were dissolved in PhMe (c=0.3 M). The reaction mixture was heated under reflux until full consumption of the substrates (water was collected in a Dean-Stark apparatus). The reaction mixture was cooled down to room temperature (RT) and the solvent was evaporated under reduced pressure, the crude reaction mixture was filtered on a short pad of neutral aluminium oxide (Al2O₃, neutral, Broockman Grade 1), dried in vacuo, obtaining an imine which was used in the next step without further purification.

### Method B

In a round-bottom flask equipped with a magnetic stirring element, the appropriate aldehyde (1.00 equiv.) was dissolved in anh. DCM (c=0.5 M) and 4 Å molecular sieves were added, followed by the addition of aniline (1.00 equiv.). The obtained solution was stirred at room temperature for 16 hours. The reaction mixture was filtered through neutral Celite, the solvent was evaporated under reduced pressure. The residue was dried in vacuo, obtaining imine which was used in the next step without further purification.

### General Methods for the Imine Alkylation Reaction

Under a protective argon atmosphere, in a Schlenk flask equipped with a magnetic stirring element, the imine (1.00 equiv.) was dissolved in anh. THE (c=0.5 M) and cooled to −78° C. BuLi (1.20 equiv.) was added dropwise and the obtained solution was stirred for 10 minutes at −78° C., heated to room temperature and stirred for an additional hour. The reaction mixture was cooled down to −20° C., and 3-chloro-2-methylpropene (1.50 equiv.) was added. Next, the flask was heated up to room temperature and stirring continued for 16 hours. The solvent and volatile substances were evaporated under reduced pressure and dried in vacuo. The crude reaction mixture was dissolved in n-hexane and passed through a short layer of neutral aluminium oxide (Al₂O₃, neutral, Broockman Grade 1). The solvent was evaporated under reduced pressure and the residue was dried in vacuo, resulting in an imine which was used in the next step without further purification.

### General Method of Witting Reaction

Under a protective argon atmosphere, in a round-bottom flask equipped with a magnetic stirring element, (methoxymethyl)triphenylphosphonium chloride (1.60 equiv.) was suspended in anh. THF (c=0.5 M) and the obtained mixture was cooled down to 0° C., followed by the addition of solid t-BuOK (1.60 equiv.). The obtained solution was stirred at 0° C. for 1 hour, next at room temperature for 30 minutes, and during this time the solution changed colour to dark red. Next, the reaction mixture was cooled down to 0° C. and the appropriate ketone (1.00 equiv.) was dissolved in anh. THE was added dropwise and the mixture was stirred for 16 hours. The solvent was evaporated under reduced pressure and n-heptane was added, the mixture was stirred for 30 min. The obtained phosphine oxide was filtered through a short neutral Celite layer. The crude product was purified using the combined flash chromatography method (SiO₂, 0→5% EtOAc/n-hexane), obtaining the product.

### General Method of Aldehyde Synthesis

Enolic ether (1.00 equiv.) was dissolved in a 4:1 mixture of acetone and H₂O and the obtained solution was cooled down to 0° C. HBr (48%) (1.00 equiv.) was added dropwise next and the reaction mixture was stirred at room temperature until the substrate was fully consumed (48 hours). The solvent was removed under reduced pressure and the remaining aqueous residue was neutralised with NaHCO₃(aq.), as defined using an indicator paper (pH=8). This aqueous solution was extracted with DCM (3×10 mL). The organic layers were combined and dried over MgSO₄. The solvent was evaporated under reduced pressure and the residue was dried in vacuo, resulting in an aldehyde which was used in the next step without further purification.

General synthesis protocol for CAAC-H×BF₄Under protective argon atmosphere in a round bottom flask equipped with magnetic stirring element imine (1.00 equiv.) was dissolved in PhMe (c=0.5 M), followed by addition of HCl (c=4.0 M in dioxane) (2.50 equiv.) at 0° C. Reaction mixture was stirred at 85° C. for 16 hours, cooled to RT, and solvent was evaporated under reduced pressure. Crude product was dis-solved in DCM (10 mL), and NaBF4 (2.00 equiv.) dissolved in H2O (10 mL) was added, and the mixture was stirred at RT for 2 hours. Reaction mixture was transferred to separation funnel and extracted with DCM (3×10 mL), organic layers were combined, and dried over MgSO4. Solvent was evaporated under reduced pressure and crude product was dissolved in small amount of MeOH (1-2 mL) followed by addition of Et2O. Precipitate was formed and filtered, dried in vacuo to afford final product.

### Synthesis of CAAC Ligand Precursors

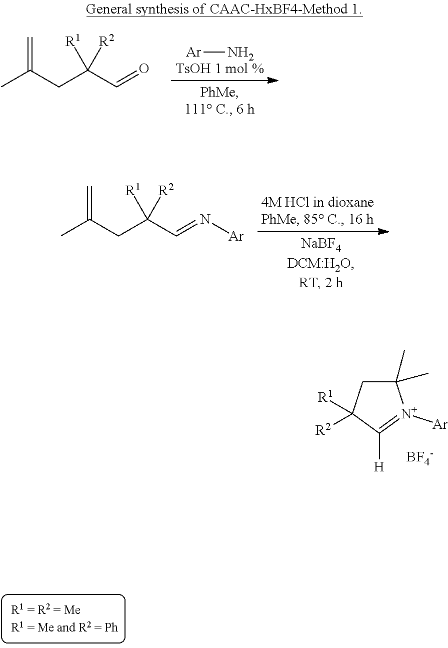

### Synthesis of 1-(2-(tert-butyl)phenyl)-2,2,4-trimethyl-4-phenyl-3,4-dihydro-2H-pyrrol-1-ium tetrafluoroborate (9bB)

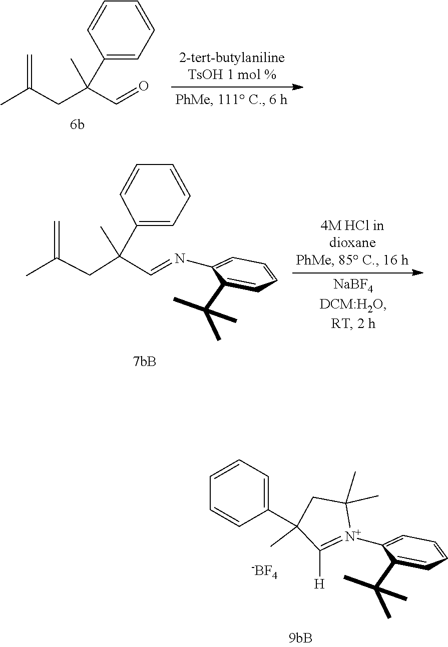

### N-(2-(tert-butyl)phenyl)-2,4-dimethyl-2-phenylpent-4-en-1-imine (7bB)

The imine (7bB) was synthesised according to the “General imine synthesis procedure”. 2,4-dimethyl-2-phenylpent-4-enal (1.85 g, 9.9 mmol), 2-(tert-butyl)aniline (1.50 g, 1.57 mL, 9.9 mmol) and PTSA (17 mg, 0.10 mmol) were used to obtain a product as a yellow oil (1.50 g, 4.7 mmol, 48%).

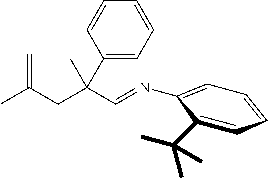

¹H NMR (400 MHz, Chloroform-d): δ 7.86-7.80 (m, 1H), 7.45-7.31 (m, 5H), 7.30-7.22 (m, 1H), 7.21-7.08 (m, 2H), 6.71-6.62 (m, 1H), 4.86-4.79 (m, 1H), 4.70-4.63 (m, 1H), 2.92-2.84 (m, 2H), 1.67-1.60 (m, 3H), 1.49-1.41 (m, 9H), 1.43-1.36 (m, 3H); ¹³C NMR (101 MHz, Chloroform-d): δ 167.8, 151.2, 144.2, 142.8, 142.7, 128.6, 127.3, 127.1, 126.7, 126.1, 125.4, 119.9, 115.2, 47.6, 47.5, 35.7, 30.5, 24.6, 22.3.

### Tetrafluoroboran1-(2-(tert-butyl)phenyl)-2,2,4-trimethyl-4-phenyl-3,4-dihydro-2H-pyrrol-1-ium (9bB)

CAAC-H×BF₄ (9bB) was synthesised according to the “General synthesis protocol for CAAC-H×BF₄”. N-(2-(tert-butyl)phenyl)-2,4-dimethyl-2-phenylpent-4-en-1-imine (1.50 g, 4.69 mmol), HCl (3.02 g, 2.93 mL, 11.7 mmol) and NaBF₄ (1.03 g, 9.39 mmol) were used to obtain the product as colourless solid (1.00 g, 2.46 mmol, 52%).

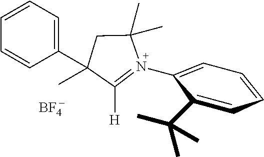

¹H NMR (400 MHz, Chloroform-d): δ 9.80 (s, 0.61×1H), 9.37 (s, 0.39×1H), 7.74 (dd, J=8.3, 1.4 Hz, 0.61×1H), 7.68-7.60 (m, 0.39×1H), 7.57-7.27 (m, 7H+0.39×1H), 6.82 (dd, J=8.1, 1.4 Hz, 0.61×1H), 3.21 (d, J=13.9 Hz, 0.61×1H), 3.06 (d, J=13.6 Hz, 0.39×1H), 2.78 (d, J=13.5 Hz, 0.39×1H), 2.56 (d, J=13.9 Hz, 0.61×1H), 1.99 (s, 0.39×3H), 1.86 (s, 0.39×3H), 1.84 (s, 0.61×3H), 1.52 (2s, 3H), 1.46 (s, 0.61×9H), 1.35 (s, 0.61×3H), 1.14 (s, 0.39×9H); ¹³C NMR (101 MHz, Chloro-form-d): δ 189.1, 187.6, 145.1, 144.4, 140.8, 140.1, 132.4, 131.5, 131.4, 131.3, 131.3, 130.2, 129.9, 128.6, 128.3, 128.2, 127.4, 127.2, 126.8, 125.7, 125.7, 82.6, 81.8, 55.3, 55.2, 48.7, 47.1, 38.1, 37.6, 33.8, 33.7, 30.3, 29.9, 28.4, 28.2, 27.2, 26.0; IR (film): 3059, 2971, 2937, 2876, 1643, 1462, 1265, 1037, 731, 700; HRMS-ESI ([M]⁺): calculated for C₂₃H₃₀N⁺: 320.2373, found: 320.2371; Elemental analysis: calculated for C₂₃H₃₀BF₄N: C, 67.82; H, 7.42; N, 3.44; found: C, 67.63; H, 7.35; N, 3.37.

### Synthesis of 2,2,4-trimethyl-1-(2-(tert-pentyl)phenyl)-4-phenyl-3,4-dihydro-2H-pyrrol-1-ium tetrafluoroborate (9bC)

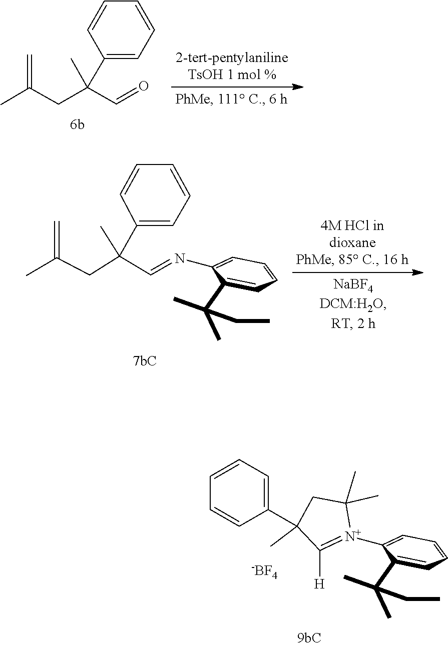

### 2,4-dimethyl-N-(2-(tert-pentyl)phenyl)-2-phenylpent-4-en-1-imine (7bC)

The imine (7bC) was synthesised according to the “General imine synthesis protocols”. The product was obtained as an orange-yellow oil (0.42 g, 1.3 mmol, 28%).

¹H NMR (400 MHz, Chloroform-d): δ 7.77 (s, 1H), 7.40-7.32 (m, 4H), 7.31-7.22 (m, 2H), 7.18-7.08 (m, 2H), 6.64-6.60 (m, 1H), 4.84-4.79 (m, 1H), 4.68-4.63 (m, 1H), 2.90-2.80 (m, 2H), 1.86 (qd, J=7.5, 1.5 Hz, 2H), 1.61 (s, 3H), 1.41 (s, 3H), 1.40-1.38 (m, 3H), 1.37 (s, 3H), 0.57 (t, j=7.5 Hz, 3H); ¹³C NMR (101 MHz, Chloroform-d): δ 167.5, 151.3, 144.3, 142.7, 140.9, 128.6, 127.5, 127.2, 127.0, 126.7, 125.2, 119.8, 115.2, 47.5, 47.4, 39.3, 34.0, 28.6, 28.4, 24.6, 22.2, 9.5.

### 2,2,4-trimethyl-1-(2-(tert-pentyl)phenyl)-4-phenyl-3,4-dihydro-2H-pyrrol-1-ium tetrafluoroborate (9bC)

CAAC-H×BF₄ (9bC) was synthesised according to the “General synthesis protocol for CAAC-H×BF₄”. N-(2-(tert-pentyl)phenyl)-2,4-dimethyl-2-phenylpent-4-en-1-imine (0.80 g, 2.40 mmol), HCl (1.54 g, 1.50 mL, 6.0 mmol) and NaBF₄ (0.53 g, 4.8 mmol) were used to obtain the product as colourless solid (0.4 g, 2.46 mmol, 40%).

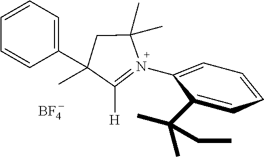

¹H NMR (400 MHz, Chloroform-d): δ 9.69 (s, 0.53×1H), 9.30 (s, 0.47×1H), 7.67 (dd, J=8.3, 1.4 Hz, 0.53×1H), 7.59-7.27 (m, 8H), 6.86 (dd, J=8.1, 1.4 Hz, 0.53×1H), 3.22 (d, J=13.9 Hz, 0.53×1H), 3.07 (d, J=13.5 Hz, 0.47×1H), 2.79 (d, J=13.5 Hz, 0.47×1H), 2.57 (d, J=13.9 Hz, 0.53×1H), 1.98 (s, 0.47×3H), 1.94-1.81 (m, 0.53×1H+0.53×3H+0.47×3H), 1.78-1.62 (m, 1H), 1.54-1.50 (m, 3H), 1.49-1.41 (m, 0.47×1H), 1.40-1.37 (m, 3H), 1.36 (0.53×3H), 1.12 (s, 0.47×3H), 0.89 (s, 0.53×3H), 0.79 (t, J=7.4 Hz, 0.52×3H), 0.57 (t, J=7.4 Hz, 0.48×3H); ¹³C NMR (101 MHz, Chloroform-d): δ 189.0, 187.5, 144.2, 143.3, 140.9, 140.3, 132.7, 132.6, 132.2, 131.6, 131.4, 131.3, 130.2, 130.0, 128.6, 128.3, 128.0, 127.4, 127.2, 126.9, 125.8, 125.6, 83.0, 81.9, 55.4, 55.1, 48.8, 47.1, 41.5, 41.0, 39.4, 39.4, 31.8, 31.1, 30.5, 30.2, 29.8, 29.7, 28.5, 28.1, 27.1, 26.2, 9.3, 9.2; IR (film): 3056, 2979, 2938, 2883, 1646, 1602, 1498, 1489, 1480, 1469, 1463, 1446, 1376, 1397, 1368, 1352, 1341, 1287, 1228, 1189, 1172, 1113, 1054, 1028, 1020, 889, 777, 761, 748, 694, 677, 656, 611, 564, 539, 520, 501; HRMS-ESI ([M]⁺): calculated for C₂₄H₃₂N⁺: 334.2529, found: 334.2526; Elemental analysis: calculated for C₂₄H₃₂BF₄N: C, 68.42; H, 7.66; N, 3.32; found: C, 68.19; H, 7.70; N, 3.29.

### Synthesis of 1-(2,5-di-tert-butylphenyl)-2,2,4-trimethyl-4-(naphthalen-1-yl)-3,4-dihydro-2H-pyrrol-1-ium tetrafluoroborate (9cD)—according to method B

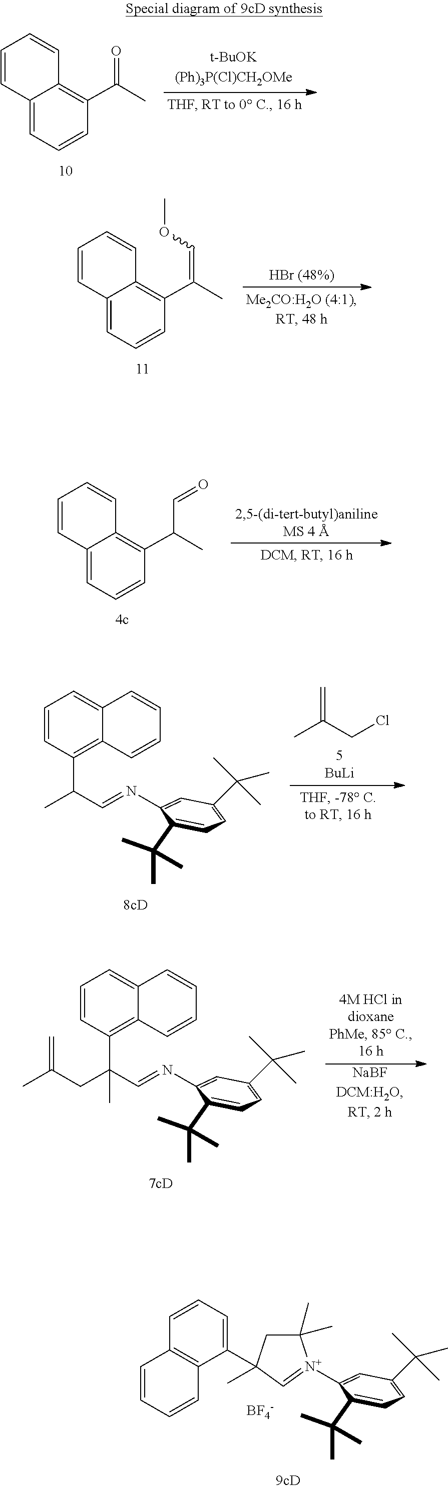

### Synthesis of 1-(1-methoxyprop-1-en-2-yl)naphthalene

The enolic ether was synthesised according to the “General Wittig reaction protocols” 1′-acetonaphtone (10.00 g, 9.00 mL, 57.0 mmol), potassium tert-butoxide (7.83 g, 68.4 mmol) and (methoxymetyl)trifphenyofosfoniym chloride (28.2 g, 79.8 mmol) with the product obtained as colourless oil (11.1 g, 55.8 mmol, 98%).

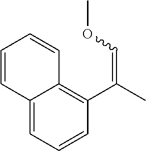

¹H NMR (400 MHz, Chloroform-d): δ 8.23-8.17 (m, 0.75×1H), 8.11-8.06 (m, 0.25×1H), 7.99-7.93 (m, 1H), 7.89-7.84 (m, 1H), 7.63-7.51 (m, 3H), 7.48-7.42 (m, 1H), 6.33 (q, J=1.4 Hz, 0.25×1H), 6.24 (q, J=1.4 Hz, 0.76×1H), 3.81 (s, 0.75×3H), 3.60 (s, 0.25×3H), 2.25 (d, J=1.5 Hz, 0.75×3H), 2.12 (d, J=1.5 Hz, 0.25×3H); ¹³C NMR (101 MHz, Chloroform-d): δ 146.2, 143.6, 139.6, 138.1, 133.9, 133.9, 132.5, 131.0, 128.4, 128.4, 127.2, 127.1, 126.4, 126.0, 126.0, 125.8, 125.8, 125.7, 125.7, 125.7, 125.6, 125.5, 113.9, 112.3, 59.7, 59.6, 20.5, 16.2.

### 2-(naphthalen-1-yl)propanal

The aldehyde was synthesised according to the “General aldehyde synthesis protocols”. 1(1-methoxyprop-1-en-2-yl)naphthalene (5.00 g, 25.2 mmol) and HBr (4.25 g, 2.85 mL, 25.2 mmol) were used, and the product was obtained as colourless oil (4.6 g, 24.9 mmol, 98%).

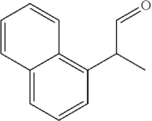

¹H NMR (400 MHz, Chloroform-d): δ 9.77 (d, J=1.3 Hz, 1H), 8.06-7.99 (m, 1H), 7.95-7.88 (m, 1H), 7.87-7.80 (m, 1H), 7.62-7.46 (m, 3H), 7.30 (dd, J=7.2, 1.2 Hz, 1H), 4.39 (qd, J=7.0, 1.3 Hz, 1H), 1.60 (d, J=7.0 Hz, 3H); ¹³C NMR (101 MHz, Chloroform-d): δ 201.4, 134.3, 134.3, 131.9, 129.3, 128.4, 126.8, 126.1, 125.8, 125.8, 123.1, 49.0, 14.8.

### N-(2,5-di-tert-butylphenyl)-2-(naphthalen-1-yl)propane-1-imine (8cD)

The imine was synthesised according to the “General imine synthesis protocols” (Method B). 2-(naphthalen-1-yl)propanal (4c) (0.70 g, 3.8 mmol, 1.00 equiv.), 2,5-di-tert-butylaniline (0.79 g, 3.8 mmol, 1.00 equiv.) were used to obtain the product as a yellow oil (1.41 g, 3.8 mmol, >99%). The imine was directly used in the subsequent stage without further purification.

### N-(2,5-di-tert-butylphenyl)-2,4-dimethyl-2-(naphthalen-1-yl)pent-4-en-1-imine (7cD)

The imine was synthesised according to the “General imine alkylation protocol”. The imine (8cD) (1.61 g, 4.34 mmol, 1.00 equiv.), 3-chloro-2-methylpropene (0.60 g, 0.66 mL, 6.51 mmol, 1.50 equiv.) and BuLi (1.54 g, 2.22 mL, 5.21 mmol, 1.20 equiv.) were used to obtain the product as yellow oil (1.50 g, 3.52 mmol, 81%). The imine was directly used in the subsequent stage without further purification.

### Synthesis of 1-(2,5-di-tert-butylphenyl)-2,2,4-trimethyl-4-(naphthalen-1-yl)-3,4-dihydro-2H-pyrrol-1-ium tetrafluoroborate (9cD)

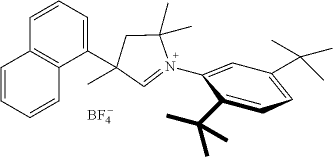

CAAC-H×BF₄ was synthesised according to the “General synthesis protocol for CAAC-H×BF₄”. N-(2,5-di-tert-butylphenyl)-2,4-dimethyl-2-(naphthalen-1-yl)pent-4-en-1-imine (1.36 g, 3.2 mmol), HCl (2.06 g, 2.00 mL, 7.99 mmol) and NaBF4 (0.70 g, 6.4 mmol) were used to obtain the product as a whitish solid (0.20 g, 0.39 mmol, 12%).

¹H NMR (400 MHz, Chloroform-d): δ 9.98 (s, 0.75×1H), 9.84 (s, 0.25×1H), 8.20 (d, J=8.7 Hz, 0.25×1H), 8.07-8.00 (m, 0.75×1H), 7.97-7.89 (m, 1H), 7.87-7.79 (m, 1H), 7.68 (d, J=8.7 Hz, 0.75×1H), 7.64-7.49 (m, 3H+0.25×1H+0.25×1H), 7.48-7.40 (m, 1H), 7.37-7.27 (m, 1H), 6.84 (d, J=2.1 Hz, 0.75×1H), 3.48 (d, J=13.4 Hz, 0.75×1H), 3.33 (d, J=13.5 Hz, 0.25×1H), 3.06 (d, J=13.5 Hz, 0.25×1H), 2.91 (d, J=13.5 Hz, 0.75×1H), 2.30 (s, 0.25×3H), 2.16 (s, 0.75×3H), 1.89 (s, 0.25×3H), 1.52 (s, 0.75×3H), 1.47 (s, 0.25×3H+0.75×9H), 1.36 (s, 0.25×9H), 1.32 (s, 0.75×9H), 1.25 (s, 0.75×3H), 1.18 (s, 0.75×3H); ¹³C NMR (101 MHz, Chloroform-d): δ 190.2, 187.2, 151.0, 150.5, 142.0, 141.3, 137.9, 136.7, 135.6, 135.4, 132.1, 131.1, 131.0, 131.0, 130.5, 130.2, 130.0, 129.7, 129.5, 128.9, 128.9, 128.7, 127.1, 126.8, 126.3, 126.2, 125.6, 125.5, 125.3, 124.4, 123.7, 123.4, 123.4, 82.8, 81.3, 55.8, 55.1, 50.2, 50.0, 37.6, 37.1, 34.7, 34.6, 33.7, 33.7, 31.0, 30.9, 30.6, 29.2, 27.4, 27.3, 26.2, 25.8; IR (film): 3079, 2963, 2873, 1652, 1472, 1392, 1383, 1378, 1371, 1362, 1340, 1291, 1267, 1193, 1064, 1019, 862, 844, 826, 803, 796, 784, 694, 674, 646, 638, 620, 610, 575, 534, 522, 478, 466, 429; HRMS-ESI ([M]⁺): calculated for: [C₃₁H₄₀N+]426.3155, found 426.3152; Elemental analysis: calculated for C₃₁H₄₀BF₄N: C, 72.51; H, 7.85; N, 2.73; found: C, 72.41; H, 7.73; N, 2.98.

### Example IX

General synthesis protocol for [Ru]-CAAC complexes. Under a protective argon atmosphere, in a Schlenk flask equipped with a magnetic stirring element, CAAC-H×BF₄ (1.60-2.20 equiv.) and (Hov-I) (1.00 equiv.) were suspended in THF (c_{[CAAC-H×BF4}]=0.1 M) and stirred for 1 minute. LiHMDS (1.60-2.20 equiv.) was then added and stirred until complete consumption of Hov-1 The crude mixture was filtered through a short layer of neutral aluminium oxide (Al2O₃, neutral, Broockman Grade 1) with Et₂O as the eluent. The green fraction was collected and evaporated under reduced pressure. Next, a small quantity (1-2 mL) of n-pentane was added and the mixture was placed in an ultrasound bath. The product was filtered and washed with cold n-pentane (1 mL). Green, crystalline solid was obtained after drying in vacuo.

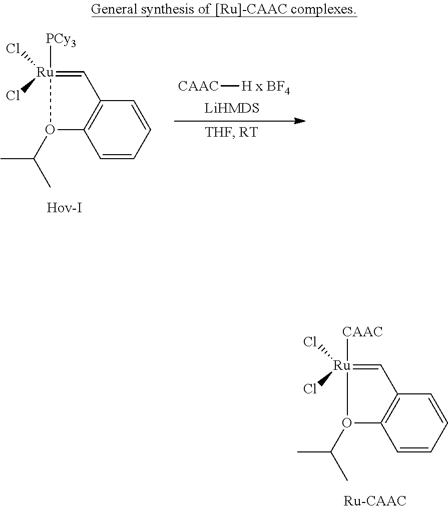

In some cases, the carbon atom of Ru=CHAr appears as a doublet in ¹³C NMR spectra. This is caused by certain technical limitations of the NMR spectrometer, which cause the {C,H}decoupling to be sometimes incorrect in the 300 ppm range, which may cause coupling between the alkylidene proton and the alkylidene carbon atom, resulting in the appearance of a doublet.

### Synthesis of the Ru16 Complex

The complex was synthesised according to the “General synthesis protocol for [Ru]-CAAC complexes”. Ru1 (200 mg, 0.33 mmol), LiHMDS (139 mg, 0.83 mmol) and CAAC-H×BF₄ (298 mg, 0.73 mmol) were used to obtain the product as a green powder (178 mg, 0.28 mmol, 84%).

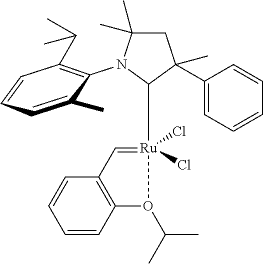

¹H NMR (400 MHz, Chloroform-d): δ 16.36 (bs, 0.36×1H), 16.34 (bs, 0.64×1H), 8.32-8.16 (m, 2H), 7.60-7.44 (m, 5H), 7.38 (q, J=7.7 Hz, 1H), 7.33-7.22 (m, 1H), 5.05-4.85 (m, 1H), 3.22-2.94 (m, 2H), 2.45-2.34 (m, 4H), 2.28 (d, J=21.6 Hz, 3H), 1.61 (s, 0.64×3H), 1.57-1.50 (m, 3H+0.54×3H), 1.49-1.44 (m, 3H), 1.42 (s, 0.36×3H), 1.35 (dd, J=6.0 Hz, 3H), 1.26 (d, J=6.6 Hz, 0.54×3H), 0.84 (d, J=6.5 Hz, 0.54×3H), 0.55 (bs, 0.46×3H); ¹¹C NMR (101 MHz, Chloroform-d): δ 301.3, 300.4, 264.7, 264.1, 152.6, 152.6, 148.9, 148.9, 144.2, 143.9, 143.4, 142.2, 138.5, 138.4, 138.4, 138.2, 131.2, 131.1, 129.9, 129.8, 129.5, 129.3, 129.3, 129.2, 129.2, 128.8, 127.7, 127.5, 126.2, 125.6, 124.2, 124.0, 121.9, 121.9, 113.4, 113.4, 78.2, 78.0, 74.7, 63.6, 63.1, 49.4, 48.2, 32.3, 31.3, 28.8, 28.8, 28.3, 28.2, 27.5, 27.4, 26.9, 25.8, 24.2, 24.2, 22.7, 22.6, 22.5, 22.2, 22.1, 21.2.

### Synthesis of the Ru-k Complex

The complex was synthesised according to the “General synthesis protocol for [Ru]-CAAC complexes”. Ru1 (50 mg, 0.08 mmol), CAAC-H×BF₄ (9bB) (75 mg, 0.18 mmol) and LiHMDS (30.6 mg, 0.18 mmol) was used to obtain the product as a green powder (40 mg, 0.06 mmol, 75%).

The complex was synthesised according to the “General synthesis protocol for [Ru]-CAAC complexes”. Ru1 (0.25 g, 0.42 mmol), CAAC-H×BF₄ (9bB) (0.37 g, 0.92 mmol) and LiHMDS (0.16 g, 0.92 mmol) were used to obtain the product as a green powder (242 mg, 0.39 mmol, 91%).

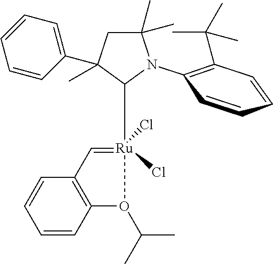

¹H NMR (400 MHz, Chloroform-d): δ 18.00 (s, 1H), 8.65 (dd, J=7.8, 1.6 Hz, 1H), 7.77 (dd, J=8.1, 1.7 Hz, 1H), 7.73-7.66 (m, 2H), 7.58-7.46 (m, 2H), 7.43 (td, J=7.5, 1.6 Hz, 1H), 7.37-7.30 (m, 2H), 7.29-7.23 (m, 1H), 7.18 (dd, J=7.6, 1.6 Hz, 1H), 6.94 (d, J=8.4 Hz, 1H), 6.91-6.86 (m, 1H), 5.05 (sep, J=6.2 Hz, 1H), 2.58-2.40 (m, 2H), 2.06 (s, 3H), 1.57-1.52 (m, 12H), 1.49 (d, J=6.2 Hz, 3H), 1.43-1.39 (m, 6H); ¹³C NMR (101 MHz, Chloroform-d): δ 298.79, 298.37, 273.96, 153.81, 153.80, 150.57, 148.74, 143.84, 143.82, 138.37, 133.58, 131.76, 131.15, 129.28, 128.48, 126.65, 126.62, 126.50, 123.64, 122.04, 113.30, 74.82, 74.59, 64.16, 55.61, 37.52, 33.95, 33.14, 28.66, 27.52, 22.07, 21.75; IR: 3076, 3063, 3031, 2987, 2973, 2962, 2952, 2936, 2871, 1589, 1577, 1494, 1489, 1473, 1454, 1396, 1383, 1374, 1315, 1301, 1286, 1246, 1223, 1158, 1139, 1119, 1097, 1075, 1059, 1038, 1027, 1013, 1003, 939, 894, 880, 846, 816, 764, 709, 694, 569, 537, 443, 750; HRMS-ESI ([M]⁺): calculated for C₃₃H₄₁NOCl₂Ru⁺: 639.1603, found: 639.1600; Elemental analysis: calculated for C₃₃H₄₁Cl₂NORu: C, 61.96; H, 6.46; N, 2.19; found: C, 61.84; H, 6.45; N, 2.26.

### Synthesis of the Ru-m Complex

The complex was synthesised according to the “General synthesis method for [Ru]-CAAC complexes”. Ru1 (150 mg, 0.25 mmol), CAAC-H×BF₄ (9bC) (231 mg, 0.55 mmol) and LiHMDS (95 mg, 0.55 mmol) were used to obtain the product as a green powder (132 mg, 0.20 mmol, 81%).

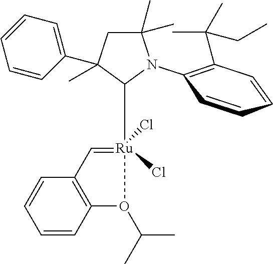

¹H NMR (400 MHz, Chloroform-d): δ 18.00 (d, J=1.0 Hz, 0.94×1H), 16.82 (s, 0.06×H), 8.65 (dd, J=7.8, 1.7 Hz, 0.95×1H), 8.23 (d, J=7.7 Hz, 0.05×1H), 7.81-7.66 (m, 3H), 7.59-7.40 (m, 3H) 7.37-7.30 (m, 2H), 7.28-7.23 (m, 1H), 7.19-7.10 (m, 1H), 6.96-6.82 (m, 2H), 5.04 (sep, J=6.2 Hz, 0.95×1H), 4.98-4.89 (m, 0.05×1H), 2.53-2.38 (m, 2H), 2.06 (s, 3H), 1.92-1.85 (m, 2H), 1.56-1.53 (m, 3H), 1.51-1.44 (m, 9H), 1.41 (d, J=6.1 Hz, 3H), 1.38 (s, 3H), 0.89 (t, J=7.5 Hz, 3H); ¹³C NMR (101 MHz, Chloroform-d): δ 298.8, 298.4, 274.1, 153.9, 150.6, 149.9, 143.9, 143.9, 138.5, 133.7, 132.0, 131.2, 131.2, 129.2, 128.5, 126.7, 126.6, 126.5, 123.7, 122.0, 113.3, 74.8, 74.6, 64.2, 55.8, 40.5, 37.3, 33.2, 29.8, 28.7, 27.6, 27.6, 22.1, 21.8, 9.1; IR: 3064, 3032, 2971, 2949, 2873, 1590, 1578, 1474, 1456, 1394, 1383, 1374, 1302, 1245, 1222, 1159, 1119, 1100, 1013, 941, 881, 846, 815, 751, 707, 693, 568, 537, 400; HRMS-ESI ([M]⁺): calculated for C₃₄H₄₃NOCl₂Ru⁺: 653.1760, found: 639.1761; Elemental analysis: calculated for [C₃₄H₄₃NCl₂RuO]×[0.5 CH₂Cl₂]: C, 59.52; H, 6.37; N, 2.01; found: C, 59.68; H, 6.47; N, 1.96.

### Synthesis of the Ru-n Complex

The complex was synthesised according to the “General synthesis protocols for [Ru]-CAAC complexes”. Ru1 (129 mg, 0.21 mmol), CAAC-H×BF₄ (9cD) (176 mg, 0.34 mmol) and LiHMDS (59 mg, 0.34 mmol) were used and the product was obtained as green powder (130 mg, 0.17 mmol, 81%).

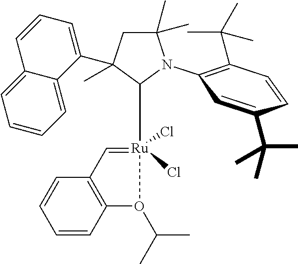

¹H NMR (400 MHz, Chloroform-d) δ 18.34 (s, 0.7×1H), 18.18 (s, 0.3×1H), 8.72-8.65 (m, 0.3×1H), 8.52-8.44 (m, 2×0.7×1H), 8.44-8.36 (m, 1H), 8.07 (d, J=2.3 Hz, 0.3×1H), 8.01-7.93 (m, 1H), 7.79-7.71 (m, 1H), 7.68 (dd, J=8.6, 2.9 Hz, 1H), 7.67-7.46 (m, 4H), 7.25-7.12 (m, 2H), 6.99-6.82 (m, 2H), 5.07 (dep, J=12.3, 6.1 Hz, 1H), 3.16 (dd, J=12.4, 2.1 Hz, 1H), 2.77 (d, J=12.1 Hz, 0.3×1H), 2.68 (d, J=12.7 Hz, 0.7×1H), 2.43 (s, 0.7×3H), 2.39 (s, 0.3×3H), 1.65-1.62 (m, 2×[0.7×3H]), 1.53 (d, J=6.1 Hz, 0.7×3H), 1.48-1.41 (m, 18H+0.3×3H), 1.38 (d, J=6.1 Hz, 0.3×3H), 1.33-1.31 (m, 2×[0.3×3H]), 1.24 (s, 0.7×3H); ¹³C NMR (101 MHz, Chloroform-d): δ 298.9, 296.5, 272.0, 269.3, 154.0, 148.9, 148.0, 145.5, 145.1, 144.6, 144.3, 144.2, 143.8, 136.9, 135.6, 135.6, 134.9, 133.0, 131.2, 131.0, 130.9, 130.7, 130.7, 130.5, 130.2, 130.1, 128.2, 128.0, 127.8, 127.1, 126.9, 126.6, 126.4, 125.3, 125.2, 124.9, 124.9, 124.8, 123.6, 123.4, 122.1, 122.0, 113.3, 113.2, 76.0, 75.2, 75.1, 74.9, 64.1, 64.0, 54.8, 54.5, 37.4, 37.3, 34.9, 34.5, 34.5, 33.9, 33.7, 32.6, 31.6, 31.3, 31.2, 31.2, 28.4, 27.0, 26.5, 22.3, 22.1, 21.5, 21.4; IR: 3070, 2961, 2869, 1591, 1578, 1506, 1474, 1456, 1397, 1388, 1361, 1303, 1230, 1116, 1099, 1026, 1020, 932, 809, 781, 742, 681, 570, 709, 1157, 443; HRMS-ESI ([M]⁺): calculated for C₄₁H₅₁NOCl₂Ru⁺: 745.2385, found: 745.2383; Elemental analysis: calculated for C₄₁H₅₁NOCl₂Ru: C, 66.03; H, 6.89; N, 1.88; found: C, 65.88; H, 6.93; N, 1.89.

### Synthesis of the Ru-o Complex

The complex was synthesised according to the “General synthesis protocol for [Ru]-CAAC complexes”. Ru1 (300 mg, 499 μmol), CAAC-H×BF₄ (9aD) (441 mg, 1.10 mmol), LiHMDS (184 mg, 1.10 mmol) and copper(I) chloride (98.9 mg, 999 μmol) were used to obtain the product as green powder (218 mg, 244 μmol, 69%).

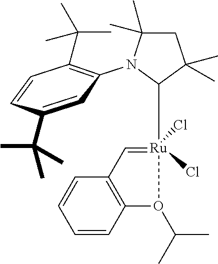

¹H NMR (400 MHz, Chloroform-d) δ 16.59 (s, 1H), 7.73 (d, J=8.6 Hz, 1H), 7.56-7.45 (m, 2H), 7.19 (s, 1H), 6.93 (d, J=8.4 Hz, 1H), 6.83 (d, J=5.9 Hz, 2H), 5.15 (hept, J=5.4 Hz, 1H), 2.28 (s, 3H), 2.16 (s, 2H), 1.86-1.76 (m, 6H), 1.68 (d, J=6.1 Hz, 3H), 1.51-1.44 (m, 12H), 1.29 (s, 3H), 1.06 (s, 9H); ¹³C NMR (101 MHz, Chloroform-d) δ 298.6, 298.4, 265.6, 152.5, 149.9, 144.3, 144.0, 139.0, 132.0, 130.6, 125.2, 123.1, 121.9, 113.2, 76.4, 74.9, 55.8, 52.1, 37.4, 34.2, 33.8, 31.1, 29.9, 28.1, 28.0, 22.4, 21.8; IR: 3069, 2972, 2954, 2939, 2868, 1627, 1610, 1589, 1577, 1502, 1476, 1455, 1440, 1432, 1398, 1385, 1376, 1363, 1316, 1294, 1268, 1237, 1224, 1199, 1159, 1129, 1112, 1095, 1087, 1068, 1037, 1019, 1012, 942, 880, 839, 806, 793, 751, 688, 657, 641, 580, 563, 485, 450, 438; HRMS-ESI ([M]⁺): calculated for C₃₂H₄₇NOCl₂Ru⁺: 633.2073, found: 633.2083.

### Example X

### Methyl Oleate Ethenolysis

### Preparation and Processing of Methyl Oleate

Oleic acid (500 g) was dissolved in acetone (1 g acid in 11 mL of the solvent). The mixture was cooled down to −40° C. and stirred for 16 hours. Solids were filtered on a Buchner funnel, transferred into a round-bottom flask, and the remaining acetone was evaporated on a rotary evaporator, followed by high vacuum. This process was repeated 2-4 times. The remaining oleic acid was dissolved in MeOH and stirred under argon for 16 hours with a catalytic amount of PTSA. After cooling down to room temperature, MgSO₄ was added and the reaction mixture was stirred for 2 hours, the solids were filtered and MeOH was evaporated under reduced pressure. Freshly prepared methyl oleate was placed in a round-bottom flask equipped with a magnetic stirring element. Activated aluminium oxide was added (2.5 wt. %) and a distillation set was assembled. The contents of the flask were heated under high vacuum at 60° C. for 1 hour before the distillation was started. 10% of the heads and 10% of the tails were discarded. The freshly distilled methyl oleate was treated with 2.5 wt % activated aluminium oxide and 0.1 mol % BHT, gases were evacuated and the contents of the flask were stirred at 100° C. for ca. 1 hour, under a gentle flow of argon. After cooling to room temperature, methyl oleate was filtered through a pad of activated aluminium oxide under argon, to a flask, and stored in a vial over activated aluminium oxide in the dark.

### FAME (Fatty Acid Methyl Ester) Preparation

FAME was treated with 2.5 wt % activated aluminium oxide and 0.1 mol % BHT, gases were evacuated and the contents of the flask were stirred at 100° C. for 1 hour, under a gentle flow of argon. After cooling to room temperature, FAME was filtered through a pad of activated aluminium oxide under argon, to a flask, and transferred and stored in a vial over activated aluminium oxide in the dark. GC FAME composition: 73.1% methyl oleate, 19.7% methyl linoleate, 1.4% methyl stearate, methyl elaidate and small quantities of other impurities.

### General Ethenolysis Procedure for Methyl Oleate and FAME Under Argon Atmosphere

A Schlenk vessel equipped with a magnetic stirring element (previously weighed with a stopper on a balance) [photograph A] was connected to the vacuum-argon line. Next, filtration funnel with a frit [photograph B] was installed in the top part of the Schlenk vessel and Al₂O₃ was poured [photographs C and C′]. The filtration funnel was evacuated for 30 minutes and filled with argon. Methyl oleate (95% purity) or FAME (containing 73% of methyl oleate and 19.7% of methyl linoleate) (15 mmol) was collected from the vial using a syringe and transferred onto the filtration funnel [photographs D and D′], filtered through a Al₂O₃ layer using pressurised argon. Next, the filtrated methyl oleate or FAME [photograph E] was degassed in vacuo (p~1×10⁻² mbar) for 10 minutes [photograph F] and the Schlenk vessel with the substrate was weighed again (the quantity of methyl oleate or FAME was calculated from the mass difference). In a separate Schlenk vessel, under argon atmosphere, a weighed sample of the catalyst (Ru-16, Ru-j, Ru-k, Ru-m, Ru-n, Ru-o) was dissolved in anhydrous PhMe (c=1 mg/mL). The appropriate quantity of the catalyst solution (corresponding to 0.5-15 ppm Ru) was added under argon atmosphere to a Schlenk vessel containing filtered methyl oleate or FAME using a Hamilton's syringe (the quantity of the dissolved catalyst was calculated precisely in reference to the weight of the substrate in the Schlenk vessel) and appropriately stirred, with the vessel being shaken every 5 seconds. The obtained mixture was immediately sucked into a steel autoclave [photograph G]—containing a glass reaction vessel equipped with a magnetic stirring element—through a Teflon tube [photographs H and I] using vacuum. The autoclave was then filled with ethylene (10 bar) and the reaction mixture was stirred at 40° C. for 6 hours. After this period of time ethylene was removed and the autoclave was disassembled. A SnatchCat solution in DCM (0.5 mL) was added to the reaction mixture and stirred for 5 minutes. A sample of the reaction mixture was then collected and subjected to GC analysis. Each sample was measured 4 times and the results are averages over 4 measurements, where the deviations between the average result and each of the measurements are 0.5-1%.

### General Ethenolysis Procedure for Methyl Oleate and FAME in the Air

The reaction configuration uses the same equipment as above, but air is used instead of argon throughout the entire procedure. A Schlenk vessel equipped with a magnetic stirring element (previous weighed with a stopper on a balance) [photograph A as above] was connected to a vacuum pump. Next, a filtration funnel with a frit was installed at the top of the Schlenk vessel [photograph B] and Al₂O₃[photographs C and C′] was poured in the air. The filtration funnel was evacuated for 30 minutes and filled with air. A sample of methyl oleate (95% purity) or FAME (containing 73% of methyl oleate and 19.7% of methyl linoleate) was collected from the vial using a syringe (Note 1) and transferred onto the filtration funnel [photographs D and D′], filtered through a Al₂O₃ layer using pressurised air. Next, the filtered methyl oleate or FAME [photograph E] was degassed in vacuo (p~1×10⁻² mbar) for 10 min [photograph F] and the Schlenk flask was weighed again (the quantity of methyl oleate or FAME was calculated from the mass difference). In a separate Schlenk vessel, in the air, a weighed sample of the catalyst (Ru-16, Ru-j, Ru-k, Ru-m, Ru-n, Ru-o) was dissolved in anhydrous PhMe (c=1 mg/mL). The appropriate quantity of the catalyst solution (corresponding to 0.5-15 ppm Ru) was added to the Schlenk vessel containing methyl oleate or FAME using a Hamilton's syringe in the air (the quantity of the dissolved catalyst was precisely calculated in relation to the weight of the substrate in the Schlenk flask) and the contents were mixed appropriately, shaking the Schlenk vessel for 5 seconds. The mixture was immediately transferred in vacuo to a steel autoclave-containing a glass reaction vessel equipped with a magnetic stirring element-using a Teflon tube. The autoclave was then filled with ethylene (10 bar) and the reaction mixture was stirred at 40° C. for 6 hours. After this period of time ethylene was removed and the autoclave was disassembled. A SnatchCat solution in DCM (2 mL) was added to the reaction chamber and the reaction mixture was stirred for 5 minutes. A sample of the reaction mixture was collected and subjected to GC analysis. Each sample was measured 4 times and the results are averages over 4 measurements, where the deviations between the average result and each of the measurements are 0.5-1%.

Note 1: For the purpose of longer storage, methyl oleate or FAME and anhydrous PhMe were stored in tightly closed vials, under argon.

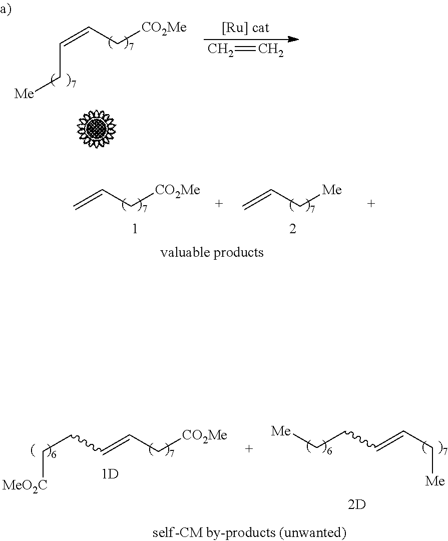

TABLE 4
Results of the ethenolysis reaction with 3 and ½ ppm of the
new catalysts.

| | Loading | Conversion | Selectivity | Capacity | |
|---|---|---|---|---|---|
| [Ru] | [ppm] | [%] | [%] | [%] | TON |
| Ru16 | 3 | 54 | 96 | 51 | 173,000 |
| | ½ | 19 | 97 | 19 | 360,000 |
| Ru-k | 3 | 83 | 88 | 73 | 244,000 |
| | ½ | 40 | 93 | 37 | 744,000 |
| Ru-m | 3 | 81 | 89 | 72 | 242,000 |
| | ½ | 29 | 93 | 27 | 536,000 |
| Ru-j | 3 | 80 | 90 | 72 | 240,000 |
| | ½ | 23 | 90 | 20 | 409,000 |
| Ru-n | 3 | 80 | 89 | 71 | 237,000 |
| | ½ | 38 | 95 | 36 | 723,000 |
| Ru-o | 3 | 56 | 35 | 20 | 65,000 |
| | ½ | 13 | 8 | 1 | 20,000 |

Conditions for 3 ppm of the catalyst: 6 h, 40° C., ethylene purity 3.5 (99.95%), 10 bar. Conditions for ½ ppm of the catalyst: 6 h, 40° C., ethylene purity 4.5 (99.995%), 10 bar. Conversion=100×[1−(A₃×A⁰_{IS})/(A⁰₃×A_{IS})]; Selectivity=100×(n₁+n₂)/[(n₁+n₂)+2×(n_{1D}+n_{2D})]; Yield=(conversion×selectivity)/100; TON=Yield×[(n⁰₃/n⁰_{[Ru]})]/100; A₃, A_{IS}=GC peak area of methyl oleate and internal standard at the end of the reaction; A⁰₃, A⁰_{IS}=GC peak area of methyl oleate and internal standard before the reaction; n⁰₃, n⁰_{[Ru]}=initial number of moles of the used methyl oleate and of the catalyst; IS=internal standard (methyl stearate).

TABLE 5
The impact of ethylene purity on the ethenolysis reaction.

| | Load- | | | | |
|---|---|---|---|---|---|
| | ing | | | | |
| | [ppm] | | | | |
| | (eth- | Con- | | | |
| | ylene | version | Selectivity | Capacity | |
| [Ru] | purity) | [%] | [%] | [%] | TON |
| Ru | ½ | 11 | 97 | 11 | 219,000 |
| 16 | (3.5) | | | | |
| | ½ | 19 | 97 | 19 | 360,000 |
| | (4.5) | | | | |
| Ru- | 2 | 75 | 91 | 68 | 340,000 |
| k | (3.5) | | | | |
| | 2 | 76 | 91 | 68 | 341,000 |
| | (4.5) | | | | |
| | 1 | 54 | 93 | 51 | 506,000 |
| | (3.5) | | | | |
| | 1 | 58 | 92 | 54 | 536,000 |
| | (4.5) | | | | |
| | ½ | 40 | 94 | 37 | 743,000 |
| | (3.5) | | | | |
| | ½ | 40 | 93 | 37 | 744,000 |
| | (4.5) | | | | |

Conditions: 6 h, 40° C., ethylene purity 3.5 (99.95%) or 4.5 (99.995%), 10 bar. The reaction was carried out under argon. Conversion=100×[1−(A₃×A⁰_{IS})/(A⁰₃×A_{IS})]; Selectivity=100×(n₁+n₂)/[(n₁+n₂)+2×(n_{1D}+n_{2D})]; Yield=(conversion×selectivity)/100; TON=Yield×[(n⁰₃/n⁰_{[Ru]})]/100; A₃, A_{IS}=GC peak area of methyl oleate and internal standard at the end of the reaction; A⁰₃, A⁰_{IS}=GC peak area of methyl oleate and internal standard before the reaction; n⁰₃, n⁰_{[Ru]}=initial number of moles of the used methyl oleate and of the catalyst; IS=internal standard (methyl stearate).

TABLE 6
The impact on time on the ethenolysis reaction.

| | | Con- | | | |
|---|---|---|---|---|---|
| | Time | version | Selectivity | Capacity | |
| [Ru] | [h] | [%] | [%] | [%] | TON |
| Ru- | 6 | 40 | 94 | 37 | 743,000 |
| k | 3 | 19 | 94 | 18 | 376,000ª |

TABLE 7
Results of ethenolysis reaction of methyl oleate in the air.

| | | Con- | | | |
|---|---|---|---|---|---|
| | Loading | version | Selectivity | Capacity | |
| [Ru] | [ppm] | [%] | [%] | [%] | TON |
| Ru16 | 3 | 49 | 96 | 48 | 158,000 |
| Ru-k | 3 | 70 | 90 | 63 | 211,000 |

Conditions: 6 h, 40° C., ethylene purity 3.5 (99.95%), 10 bar. The reaction was carried out in the air. Conversion=100×[1−(A₃×A⁰_{IS})/(A⁰₃×A_{IS})]; Selectivity=100×(n₁+n₂)/[(n₁+n₂)+2×(n_{1D}+n_{2D})]; Yield=(conversion×selectivity)/100; TON=Yield×[(n⁰₃/n⁰_{[Ru]})]/100; A₃, A_{IS}=GC peak area a of methyl oleate and internal standard at the end of the reaction; A⁰₃, A⁰_{IS}=GC peak area a of methyl oleate and internal standard before the reaction; n⁰₃, n⁰_{[Ru]}=initial number of moles of the used methyl oleate and of the catalyst; IS=internal standard (methyl stearate). ^{a}Average value for 3 reactions. Conditions: 500 Ru-k, 40° C., ethylene purity 3.5 (99.95%), 10 bar. The reaction was carried out under argon. Conversion=100×[1−(A₃×A⁰_{IS})/(A⁰₃×A_{IS})]; Selectivity=100×(n₁+n₂)/[(n₁+n₂)+2×(n_{1D}+n_{2D})]; Yield=(conversion×selectivity)/100; TON=Yield×[(n⁰₃/n⁰_{[Ru]})]/100; A₃, A_{IS}=GC peak area a of methyl oleate and internal standard at the end of the reaction; A⁰₃, A_{IS}=GC peak area a of methyl oleate and internal standard before the reaction; n⁰₃, n⁰_{[Ru]}=initial number of moles of the used methyl oleate and of the catalyst; IS=internal standard (methyl stearate).

TABLE 8
Results of FAME ethenolysis reaction in the
air and under argon.

| | Load- | Con- | | | |
|---|---|---|---|---|---|
| | ing | version | Selectivity | Capacity | |
| [Ru] | [ppm] | [%] | [%] | [%] | TON |
| Ru16 [argon] | 3 | 17 | 99 | 17 | 57,000 |
| Ru16 [air] | 15 | 65 | 96 | 63 | 42,000 |
| Ru-k [argon] | 3 | 27 | 99 | 27 | 89,000 |
| Ru-k [Argon] | 15 | 89 | 90 | 80 | 54,000 |
| Ru-k [air] | 15 | 86 | 91 | 78 | 52,000 |

Conditions: 6 h, 40° C., ethylene purity 3.5 (99.95%) Conversion=100×[1−(A_{FAME}×A⁰_{IS})/(A⁰_{FAME}×A_{IS})]; Selectivity=100×(n₁+n₂)/[(n₁+n₂)+2×(n_{1D}+n_{2D})]; Yield=(conversion×selectivity)/100; TON=Yield×[(n⁰_{FAME}/n⁰_{[Ru]})]/100; A_{FAME}, A_{IS}=GC peak area a of methyl oleate and internal standard at the end of the reaction; A⁰_{FAME}, A⁰_{IS}=GC peak area a of methyl oleate and internal standard before the reaction; n⁰_{FAME}, n⁰_{[Ru]}=the initial number of moles of the used FAME and the catalyst; IS=internal standard (methyl stearate).

### Example XI

### General Catalyst Stability Testing Procedure in the Presence of Ethylene—See FIG. 10

15 mg (0.02 mmol) of the ruthenium complex [Ru16 or Ru-k] was dissolved in 0.7 mL CD₂Cl₂ in an NMR Young tube. Next, 1 mg of 1,3,5-trimethoxybenzene (0.006 mmol) was added [internal standard] and the contents of the tube were shaken until the ruthenium catalyst and the internal standard dissolved. Once the ¹H NMR “0” spectrum was recorded at 40° C., the NMR tube with the cap removed was placed in an autoclave. The chamber of the autoclave was filled 3 times [2 minutes each] with ethylene, under a pressure of 2 bar. Next, the autoclave chamber was purged with ethylene, under the pressure of 10 bar. The chamber was subjected to dynamic pressure up to 10 bar for 20 minutes. At the end of this time, the autoclave was depressurised and opened, the NMR Young tube was removed and the contents of the tube was shaken four times and placed in the NMR apparatus (30 minutes after the first ethylene treatment) at 40° C. The spectra were recorded over 8 hours at 40° C. Decomposition was measured in reference to the disappearance of the alkylidene signal compared to the signal from the standard (OMe groups).

### Summary [Technical Effect]

It was observed that catalysts with a bulky substituent with a quaternary carbon atom at the nitrogen atom in the aromatic ring and with at least one aromatic substituent at the C2 atom in the CAAC ligand lead to a persistent configuration of the ruthenium complex, in which the N-aromatic substituent is on the side opposite to the benzylidene substituent. This was confirmed using crystallographic analysis, and the inventors believe that this configuration of the CAAC ligand is retained In the solution, judging by the characteristic shift of the Ru═CH benzylidene signals downfield near 18 ppm, [instead of a shift near 16 ppm, similar to the publication Organometallics 2023, 42, 6, 495-504]. In the solution, single signals from alkylidene can be observed in the ¹H NMR spectrum, which can indicate that an analogous ligand configuration on the opposite side of the benzylidene ligand is maintained in the solution. This spatial structure facilitates the coordination of the olefin substrate to the metallic ruthenium centre and may prevent the decomposition of the methylidene intermediate through C—H insertion, which may explain the observed, high activity and stability of these catalysts. It is noteworthy that the confirmation of the structure in crystallographic analysis, as well as the persistent CAAC ligand configuration in the solution is inverse to all the configurations recorded so far in the literature [see Angew. Chem. Int. Ed. 2007, 46, 7262-7265].

### Example XI

### Obtaining Precursors of Novel CAAC Ligands

### Imine Synthesis

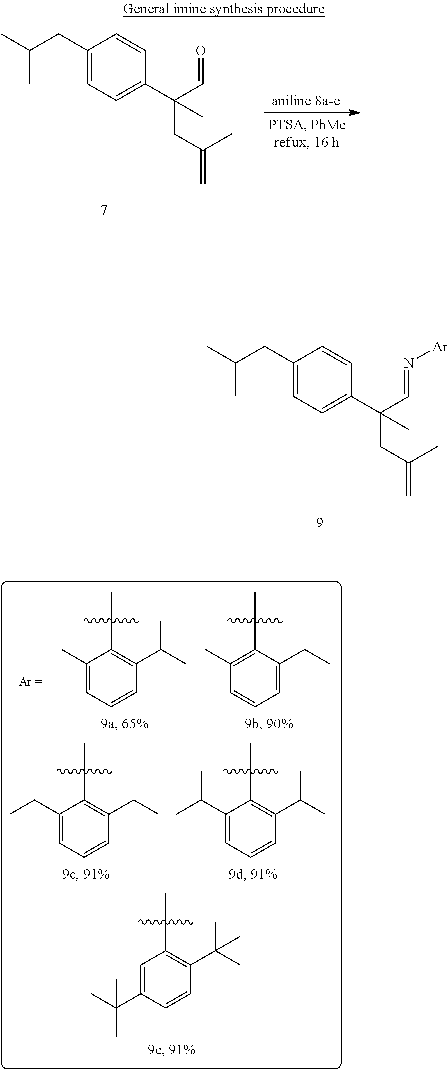

In a round-bottom flask equipped with a Dean-Stark apparatus and a magnetic stirring element, aldehyde 7 (1.00 equiv.), aniline (8a-e, 1.00 equiv.) and p-toluenesulphonic acid (PTSA, 1 mol %) were dissolved in toluene (c=0.3 M). The reaction mixture was heated to reflux under a reflux condenser, until the substrates were completely consumed (monitored using ¹H NMR measurements). The solvent was evaporated under reduced pressure, the crude reaction mixture was filtered through a short pad made of neutral aluminium oxide (Al₂O₃, neutral, Broockman Grade 1) and dried in vacuo, resulting in imines 9a-3, which were used in the subsequent step without further purification.

### N-(2-ethyl-6-methylphenyl)-2-(4-isobutylphenyl)-2,4-dimethylpent-4-en-1-imine (9b)

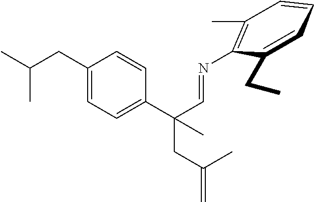

The imine was synthesised according to the “General imine synthesis procedure”. 7 (1.50 g, 6.10 mmol), 8b (830 mg, 6.10 mmol) and PTSA A (11.0 mg, 61.0 mmol) was used to obtain product 9b as yellow, oily liquid (2.00 g, 5.53 mmol, 90%).

### N-(2,6-diethylphenyl)-2-(4-isobutylphenyl)-2,4-dimethylpent-4-en-1-imine (9c)

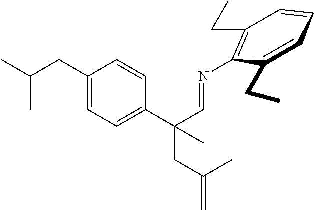

The imine was synthesised according to the “General imine synthesis procedure”. 7 (1.50 g, 6.10 mmol), 8c (916 mg, 6.10 mmol) and PTSA A (11.0 mg, 61.0 mmol) was used to obtain product 9 cas yellow, oily liquid (2.10 g, 5.60 mmol, 91%).

### N-(2,6-diisopropylphenyl)-2-(4-isobutylphenyl)-2,4-dimethylpent-4-en-1-imine (9d)

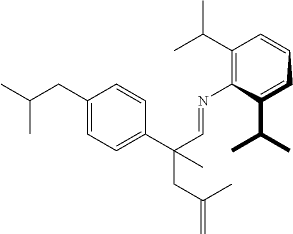

The imine was synthesised according to the “General imine synthesis procedure”. 7 (1.40 g, 5.70 mmol), 8d (1.02 g, 5.70 mmol) and PTSA A (10.0 mg, 57.0 μmol) was used to obtain product 9d as yellow, oily liquid (2.10 g, 5.20 mmol, 91%).

### N-(2,5-di-tert-butylphenyl)-2-(4-isobutylphenyl)-2,4-dimethylpent-4-en-1-imine (9e)

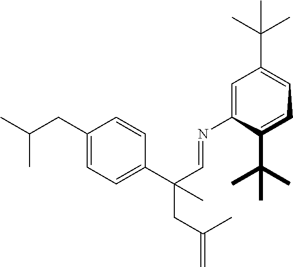

The imine was synthesised according to the “General imine synthesis procedure”. 7 (1.40 g, 5.70 mmol), 8f (1.19 g, 5.70 mmol) and PTSA A (10.0 mg, 57.0 mol) was used to obtain product 9d as yellow, oily liquid (2.25 g, 5.20 mmol, 91%).

### Synthesis of Tetrafluoroborate Salt Precursors of CAACs

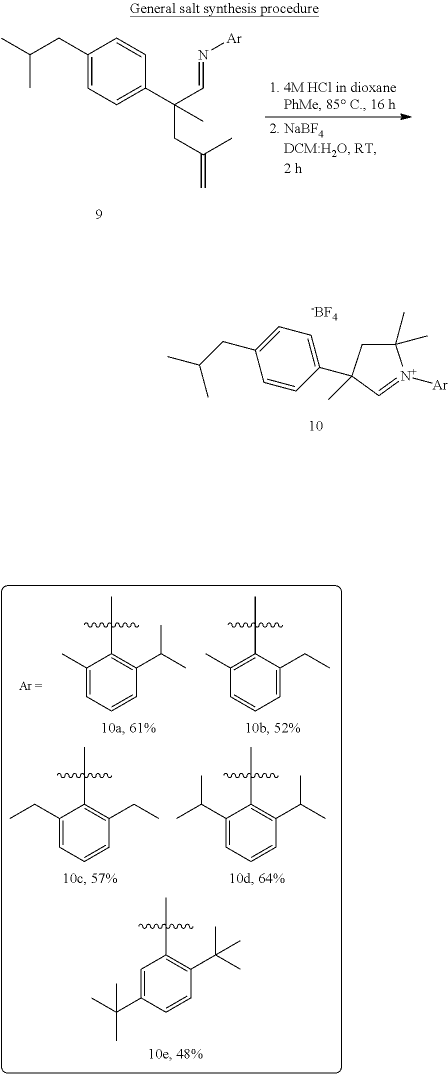

In a protective argon atmosphere, in a round-bottom flask equipped with a magnetic stirring element, the imine (9a-e, 1.00 equiv.) was dissolved in anhydrous toluene (c=0.5 M), followed by the addition of hydrochloric acid (c=4.0 M in dioxane, 2.50 equiv.) at 0° C. The reaction mixture was stirred at 85° C. for 16 hours, cooled to room temperature and the solvent was evaporated under reduced pressure. The crude product was dissolved in a small quantity of DCM, NaBF₄ (2.00 equiv.) dissolved in H₂O (ca. 10 mL) was added and the mixture was stirred at room temperature for 2 hours. The reaction mixture was transferred over to a separator and extracted thrice with DCM, the organic layers were combined, dried over anhydrous MgSO₄ and filtered on a Schott funnel, through neutral Celite. The solvent was evaporated under reduced pressure and the crude product was dissolved in a small quantity of MeOH, followed by the addition of Et₂O. A precipitate was precipitated, filtered and dried in vacuo, resulting in the final products 10a-e.

### 1-(2-ethyl-6-methylphenyl)-4-(4-isobutylphenyl)-2,2,4-trimethyl-3,4-dihydro-2H-pyrrol-1-ium tetrafluoroborate (10b)

Salt 10b was synthesised according to the “General salt synthesis procedure”. 9b (1.85 g, 5.10 mmol), hydrochloric acid (3.20 mL, 4.0 M in dioxane, 12.8 mmol) and sodium tetrafluoroborate (1.12 g, 10.2 mmol) were used to obtain the product as a colourless solid (1.22 g, 2.70 mmol, 52%).

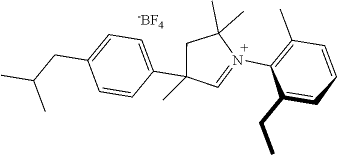

¹H NMR (400 MHz, CDCl₃): δ 9.51-9.41 (m, 1H), 7.44-7.32 (m, 3H), 7.35-7.27 (m, 0.5×H), 7.27-7.18 (m, 3H), 7.21-7.13 (m, 0.5×1H), 3.15 (ddd, J=14.0, 9.3, 1.9 Hz, 1H), 2.66 (dd, J=14.0, 2.3 Hz, 1H), 2.63-2.52 (m, 1H), 2.50-2.42 (m, 2H), 2.41-2.28 (m, 2H), 2.24-2.03 (m, 2H), 1.99-1.91 (m, 3H), 1.91-1.78 (m, 1H), 1.58 (d, J=15.1 Hz, 3H), 1.38 (s, 3H), 1.33-1.23 (m, 0.5×3H), 1.11-1.01 (m, 0.5×3H), 0.92-0.85 (m, 6H); ¹³C NMR (101 MHz, CDCl₃): δ 189.9, 142.4, 140.2, 139.6, 137.5, 137.4, 134.3, 133.2, 131.8, 131.7, 131.2, 130.8, 130.7, 130.2, 130.0, 127.9, 127.9, 125.6, 125.6, 83.9, 55.4, 55.3, 48.5, 48.4, 45.0, 45.0, 30.2, 30.2, 29.3, 28.9, 28.0, 27.5, 27.3, 26.9, 25.0, 24.8, 22.5, 22.4, 22.4, 22.4, 19.5, 19.2, 15.6, 14.8; HRMS-ESI (m/z): calculated for C₂₆H₃₆N⁺ ([M]⁺): 362.2842, found: 362.2842; Elemental analysis: calculated for C₂₆H₃₆BF₄N: C, 69.49; H, 8.08; N, 3.12; found: C, 69.59; H, 7.97; N, 3.24; IR: 3061, 2957, 2933, 2870, 1643, 1513, 1465, 1384, 1349, 1269, 1051, 1035, 797, 732, 701, 645, 573, 521.

### 1-(2,6-diethylphenyl)-4-(4-isobutylphenyl)-2,2,4-trimethyl-3,4-dihydro-2H-pyrrol-1-ium tetrafluoroborate (10c)

Salt 10c was synthesised according to the “General salt synthesis procedure”. 9c (1.85 g, 4.90 mmol), hydrochloric acid (3.08 mL, 4.0 M in dioxane, 12.3 mmol) and sodium tetrafluoroborate (1.08 g, 9.90 mmol) were used to obtain the product as a colourless solid (1.30 g, 2.80 mmol, 57%).

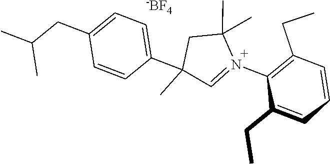

¹H NMR (400 MHz, CDCl₃): δ 9.49 (s, 1H), 7.49-7.35 (m, 3H), 7.34-7.27 (m, 1H), 7.26-7.17 (m, 3H), 3.18 (d, J=14.1 Hz, 1H), 2.64 (d, J=14.0 Hz, 1H), 2.57 (q, J=7.6 Hz, 2H), 2.49-2.44 (m, 2H), 2.32 (dq, J=15.1, 7.6 Hz, 1H), 2.13 (dq, J=14.6, 7.3 Hz, 1H), 1.93 (s, 3H), 1.91-1.78 (m, 1H), 1.54 (s, 3H), 1.33 (s, 3H), 1.28 (t, J=7.5 Hz, 3H) 1.07 (t, J=7.5 Hz, 3H), 0.88 (dd, J=6.6, 2.0 Hz, 6H); ¹³C NMR (101 MHz, CDCl₃): δ 189.8, 142.4, 140.0, 139.5, 137.5, 131.4, 130.8, 130.7, 127.9, 127.8, 125.6, 83.7, 55.3, 48.2, 45.0, 30.2, 29.1, 27.5, 26.8, 25.1, 24.8, 22.4, 22.4, 15.6, 14.7; HRMS-ESI (m/z): calculated for C₂₇H₃₈N⁺ ([M]⁺): 376.2999, found: 376.2998; Elemental analysis: calculated for C₂₇H₃₈BF₄N: C, 69.98; H, 8.27; N, 3.02; found: C, 69.85; H, 8.25; N, 3.19; IR: 3053, 2957, 2935, 2871, 1643, 1513, 1464, 1383, 1349, 1271, 1051, 1034, 798, 733, 701, 572, 520.

### 1-(2,6-diisopropylphenyl)-4-(4-isobutylphenyl)-2,2,4-trimethyl-3,4-dihydro-2H-pyrrol-1-ium tetrafluoroborate (10d)

Salt 10d was synthesised according to the “General salt synthesis procedure”. 9d (2.05 g, 5.10 mmol), hydrochloric acid (3.17 mL, 4.0 M in dioxane, 12.7 mmol) and sodium tetrafluoroborate (1.12 g, 10.2 mmol) were used to obtain the product as a colourless solid (1.60 g, 3.30 mmol, 64%).

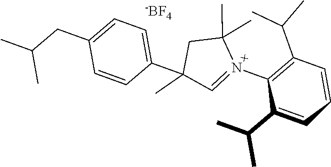

¹H NMR (400 MHz, CDCl₃): δ 9.58-9.51 (m, 1H), 7.54-7.38 (m, 3H), 7.36-7.30 (m, 1H), 7.28-7.19 (m, 3H), 3.26 (dd, J=14.1, 1.7 Hz, 1H), 2.76-2.60 (m, 2H), 2.53-2.40 (m, 2H), 2.24 (sept, J=6.8 Hz, 1H), 1.94-1.77 (m, 4H), 1.55 (bs, J=1.6 Hz, 3H), 1.38-1.34 (m, 3H), 1.31 (s, 3H), 1.23-1.17 (m, 3H), 1.13-1.09 (m, 3H), 1.06-1.01 (m, 3H), 0.91-0.86 (m, 6H); ¹³C NMR (101 MHz, CDCl₃): δ 189.9, 144.9, 144.4, 142.4, 137.3, 132.1, 130.7, 128.9, 125.7, 125.5, 83.5, 55.4, 47.7, 45.0, 30.3, 30.2, 29.2, 29.0, 27.5, 26.3, 26.2, 26.0, 22.4, 22.4, 22.3, 22.3; HRMS-ESI (m/z): calculated for C₂₉H₄₂N⁺ ([M]⁺): 404.3312, found: 202.3314; Elemental analysis: calculated for C₂₉H₄₂BF₄N: C, 70.87; H, 8.61; N, 2.85; found: C, 70.68; H, 8.40; N, 2.85; IR: 3055, 2958, 2933, 2868, 1638, 1509, 1471, 1351, 1269, 1106, 1054, 1039, 880, 808, 735, 704, 581, 566, 520.

### 1-(2,5-di-tert-butylphenyl)-4-(4-isobutylphenyl)-2,2,4-trimethyl-3,4-dihydro-2H-pyrrol-1-ium tetrafluoroborate (10e)

Salt 10e was synthesised according to the “General salt synthesis procedure”. 9e (2.25 g, 5.20 mmol), hydrochloric acid (3.26 mL, 4.0 M in dioxane, 13.0 mmol) and sodium tetrafluoroborate (1.14 g, 10.4 mmol) were used to obtain the product as a colourless solid (1.30 g, 2.50 mmol, 48%).

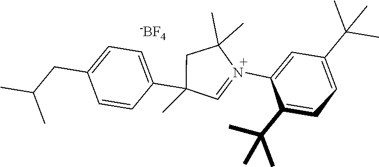

¹H NMR (400 MHz, CDCl₃): δ 9.75-9.66 (m, 0.53×1H), 9.29-9.23 (m, 0.47×1H), 7.67-7.61 (m, 0.53×1H), 7.55-7.46 (m, 1H+0.47×1H), 7.40-7.33 (m, 2H), 7.32-7.25 (m, 0.47×1H), 7.24-7.16 (m, 2H), 6.65 (bs, 0.53×1H), 3.26-3.15 (m, 0.53×1H), 3.07 (d, J=13.6 Hz, 0.47×1H), 2.76-2.67 (m, 0.47×1H), 2.53 (d, J=13.9 Hz, 0.53×1H), 2.48-2.43 (m, 2H), 2.04-1.98 (m, 0.47×3H), 1.90-1.80 (m, 4H), 1.50 (d, J=4.2 Hz, 3H), 1.46-1.41 (m, 0.53×9H), 1.36 (s, 0.53×3H), 1.35-1.32 (m, 0.47×9H), 1.27-1.24 (m, 0.53×9H), 1.11-1.08 (m, 0.47×9H), 0.91-0.85 (m, 6H); ¹³C NMR (101 MHz, CDCl₃): δ 188.6, 187.2, 150.9, 150.4, 142.2, 142.0, 141.9, 141.2, 137.9, 137.0, 132.1, 131.0, 131.0, 130.8, 130.5, 128.8, 128.5, 125.6, 125.5, 125.2, 125.1, 123.1, 82.4, 81.5, 55.0, 54.8, 48.6, 46.9, 45.0, 44.9, 37.6, 37.0, 34.6, 34.5, 33.7, 33.6, 30.9, 30.9, 30.4, 30.2, 30.1, 29.9, 28.4, 28.3, 27.1, 26.1, 22.5, 22.5, 22.4; HRMS-ESI (m/z): calculated for C₃₁H₄₆N+([M]⁺): 432.3625, found: 432.3626; Elemental analysis: calculated for C₃₁H₄₆BF₄N: C, 71.67; H, 8.93; N, 2.70; found: C, 71.44; H, 8.87; N, 2.76; IR: 3058, 2959, 2870, 1646, 1613, 1503, 1467, 1395, 1366, 1269, 1054, 848, 799, 734, 561, 520.

### Example XII

Synthesis of Novel Ruthenium Complexes with CAAC

### General Procedure of Ruthenium Complex Synthesis

In a dry Schlenk flask equipped with a magnetic stirring element, salt (10a-e, 2.20 equiv.) and a 1^{st} generation Hoveyda-Grubbs complex (Hov I) (1.00 equiv.) were suspended in THF (c_{CAAC}=0.10 M), while the mixture was stirred for 1 minute. LiHMDS (2.20 equiv.) was then added and the obtained mixture was stirred until complete consumption of the substrates. The crude mixture was next filtered through a short layer of neutral aluminium oxide (Al₂O₃, neutral, Broockman Grade 1) with Et₂O or DCM as the eluent. The green fraction was collected and the solvent was evaporated under reduced pressure. Next, a small quantity of n-pentane was added to the residue and the mixture was placed in an ultrasonic bath. The precipitate was filtered and washed with cold n-pentane (Ru-e, Ru-g) or n-heptane, followed by diethyl ether (Ru-h). Column chromatography (SiO₂, 10% AcOEt in n-hexane) was performed in order to purify the crude product to Ru-f and Ru-i, and the subsequent products were precipitated from n-pentane.

### Synthesis of the Ru-f Complex

The Ru-f complex was synthesised according to the “General synthesis method for ruthenium complexes”. Hov I (120 mg, 200 μmol), 10b (198 mg, 440 μmol) and LiHMDS (73.5 mg, 440 μmol) were used to obtained the product as a green powder (102 mg, 150 μmol, 74%).

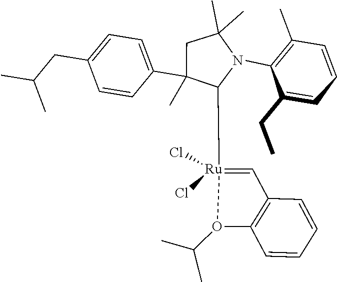

¹H NMR (400 MHz, CD₂Cl₂): δ 17.65 (s, 0.31×1H), 16.31 (s, 0.69×1H); ¹³C NMR (101 MHz, CD₂Cl₂): δ 300.8 (only the diagnostic signals of the proton and carbon of benzylidene are reported because of the presence of conformational and rotational isomers, which result in high complexity of the spectra, excluding further assignment); HRMS-ESI (m/z): calculated for C₃₆H₄₇NOCl₂Ru⁺ ([M]⁺): 681.2073, found: 681.2071; Elemental analysis: calculated for C₃₆H₄₇NOCl₂Ru: C, 63.42; H, 6.95; N, 2.05; found: C, 63.27; H, 6.95; N, 2.13; IR: 2981, 2945, 2869, 1588, 1576, 1516, 1476, 1456, 1374, 1315, 1298, 1242, 1226, 1160, 1140, 1117, 1096, 1040, 997, 932, 881, 844, 807, 778, 755, 690, 569, 544.

### Synthesis of the Ru-i Complex

The Ru-i complex was synthesised according to the “General synthesis method for ruthenium complexes”. Hov I (150 mg, 150 μmol), 10c (255 mg, 549 μmol) and LiHMDS (91.9 mg, 549 μmol) were used to obtain the product as green powder (125 mg, 180 μmol, 71%).

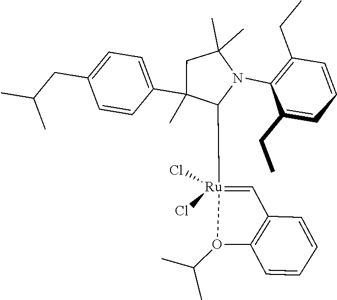

¹H NMR (400 MHz, CD₂Cl₂): δ 17.65 (bs, 0.15×1H), 16.39 (s, 0.85×1H), 8.37-7.87 (m, 2H), 7.70-7.25 (m, 6H), 6.96-6.68 (m, 3H), 4.96 (sept, J=6.1 Hz, 1H), 3.12 (d, J=12.9 Hz, 1H), 2.79-2.62 (m, 2H), 2.62-2.42 (m, 4H), 2.40-2.26 (m, 3H), 1.98-1.85 (m, 1H), 1.58-1.45 (m, 6H), 1.41-1.25 (m, 9H), 1.07-1.00 (m, 2H), 0.98-0.90 (m, 6H), 0.81-0.73 (m, 2H); ¹³C NMR (101 MHz, CD₂Cl₂): δ 299.9, 263.5, 152.5, 144.3, 143.9, 143.6, 141.4, 141.3, 139.1, 131.2, 130.4, 129.4, 128.4, 127.4, 127.2, 123.9, 122.2, 113.5, 78.3, 75.0, 63.3, 54.4, 54.1, 53.8, 53.6, 53.3, 48.4, 45.3, 31.2, 30.7, 30.1, 28.2, 27.7, 25.8, 24.5, 22.8, 22.6, 22.4, 14.9, 14.4; HRMS-ESI (m/z): calculated for C₃₇H₄₉NOCl₂Ru ([M]⁺): 695.2229, found: 695.2228; Elemental analysis: calculated for C₃₇H₄₉NOCl₂Ru: C, 63.42; H, 6.95; N, 2.05; found: C, 63.27; H, 6.95; N, 2.13; IR: 2948, 2932, 2871, 1588, 1576, 1516, 1475, 1455, 1442, 1412, 1387, 1374, 1298, 1225, 1159, 1141, 1116, 1096, 1040, 997, 932, 881, 843, 807, 792, 773, 754, 693, 602, 568, 544.

### The Ru-g Complex

The Ru-g complex was synthesised according to the “General synthesis method for ruthenium complexes”. Hov I (170 mg, 250 μmol), 10d (270 mg, 549 μmol) and LiHMDS (91.9 mg, 549 μmol) were used to obtain the product as green powder (131 mg, 181 μmol, 72%).

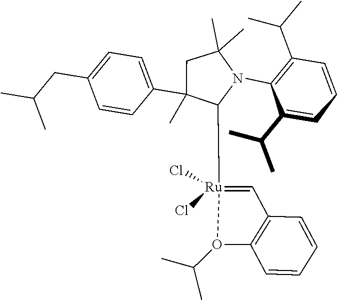

¹H NMR (400 MHz, CD₂Cl₂): δ 16.50 (s, 1H), 8.14 (d, j=8.3 Hz, 2H), 7.65 (t, J=7.8 Hz, 1H), 7.55-7.47 (m, 2H), 7.46 (dd, j=7.7, 1.6 Hz, 1H), 7.30 (d, J=8.4 Hz, 2H), 6.90 (d, J=8.4 Hz, 1H), 6.83 (td, J=7.4, 0.8 Hz, 1H), 6.72 (dd, J=7.6, 1.7 Hz, 1H), 4.95 (sept, J=6.2 Hz, 1H), 3.13 (d, J=12.8 Hz, 1H), 3.05 (sept, j=6.6 Hz, 1H), 2.94 (sept, j=6.4 Hz, 1H), 2.58 (dd, J=13.2, 6.9 Hz, 1H), 2.49 (dd, j=13.1, 7.6 Hz, 1H), 2.32 (d, j=12.8 Hz, 1H), 2.30 (s, 3H), 2.01-1.83 (m, 1H), 1.56 (d, J=6.2 Hz, 3H), 1.50 (s, 3H), 1.43-1.35 (m, 6H), 1.34 (d, J=6.6 Hz, 3H), 1.25 (d, J=6.6 Hz, 3H), 0.96 (d, J=6.7 Hz, 3H), 0.93 (d, J=6.6 Hz, 3H), 0.78 (d, J=6.5 Hz, 3H), 0.47 (d, J=6.4 Hz, 3H); ¹³C NMR (101 MHz, CD₂Cl₂): δ 296.7, 264.7, 153.0, 148.7, 148.5, 143.2, 141.2, 141.1, 137.3, 131.1, 130.3, 129.9, 128.9, 126.3, 126.1, 124.1, 122.1, 113.6, 77.9, 75.0, 63.2, 48.3, 45.3, 32.9, 30.7, 29.1, 28.8, 28.6, 28.1, 27.4, 26.5, 24.6, 24.4, 22.8, 22.6, 22.5, 22.4; HRMS-ESI (m/z): calculated for C₃₉H₅₃NOCl₂Ru⁺ ([M]⁺): 723.2542, found: 723.2537; Elemental analysis: calculated for C₃₉H₅₃Cl₂NORu: C, 64.72; H, 7.38; N, 1.94; found: C, 64.34; H, 7.43; N, 1.79; IR: 2978, 2953, 2923, 2868, 1586, 1575, 1516, 1473, 1454, 1402, 1374, 1313, 1293, 1224, 1158, 1142, 1114, 1097, 1038, 997, 936, 844, 809, 754, 569, 548.

### The Ru-h Complex

The Ru-h complex was synthesised according to the “General synthesis method for ruthenium complexes”. Hov I (300 mg, 499 μmol), 103 (571 mg, 1.10 mmol) and LiHMDS (184 mg, 1.10 mmol) were used to obtain the product as green powder (328 mg, 436 mol, 87%).

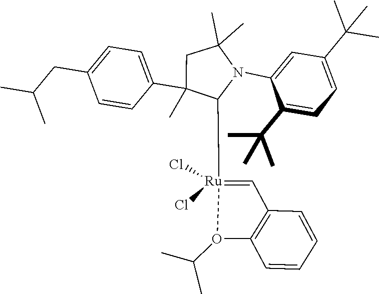

¹H NMR (400 MHz, CD₂Cl₂): δ 17.84 (s, 0.81×1H), 16.79 (s, 0.19×1H), 8.27 (d, j=2.3 Hz, 0.81×1H), 8.13 (d, J=8.1 Hz, 0.19×1H), 7.76-7.42 (m, 5H), 7.30 (d, J=8.0 Hz, 0.19×2H), 7.20 (d, J=2.3 Hz, 0.19×1H), 7.11 (d, J=8.0 Hz, 0.81×2H), 7.08 (dd, j=1.6, 7.6 Hz, 0.81×1H), 6.91 (d, j=8.4 Hz, 0.81×1H), 6.86 (td, j=0.8, 7.4 Hz, 0.81×1H), 6.78 (t, j=7.4 Hz, 0.19×1H), 6.69 (dd, j=1.7, 7.6 Hz, 0.19×H), 5.03 (sept, j=5.7 0.81×Hz, 1H), 4.93 (sept, j=5.2 Hz, 0.19×H), 3.08 (d, J=13.6 Hz, 0.19×1H), 2.61-0.84 (m, 0.81×1H+43H); ¹³C NMR (101 MHz, CD₂Cl₂): S 296.3, 296.0, 271.8, 262.5, 153.5, 152.6, 149.9, 148.8, 148.1, 145.2, 144.4, 144.1, 143.6, 143.6, 140.9, 140.1, 139.8, 139.0, 137.0, 131.8, 131.1, 131.0, 130.8, 130.8, 130.5, 130.1, 129.3, 129.2, 126.6, 126.3, 125.3, 123.4, 123.3, 121.9, 121.8, 113.4, 113.2, 76.4, 74.8, 74.7, 74.4, 64.0, 63.1, 56.0, 50.1, 45.2, 45.1, 37.5, 37.1, 34.9, 34.8, 34.2, 34.0, 33.9, 33.2, 31.6, 31.1, 30.3, 30.3, 28.6, 28.5, 28.2, 27.8, 22.9, 22.6, 22.6, 22.6, 22.5, 22.2, 21.9, 21.9; HRMS-ESI (m/z): calculated for C₄1H₅₇NOCl₂Ru⁺ ([M]⁺): 751.2855, found: 751.2857; Elemental analysis: calculated for C₄₁H₅₇NOCl₂Ru: C, 65.50; H, 7.64; N, 1.86; found: C, 65.51; H, 7.83; N, 1.99; IR: 3069, 2995, 2955, 2866, 1589, 1575, 1499, 1475, 1454, 1397, 1387, 1370, 1362, 1314, 1300, 1271, 1249, 1221, 1150, 1141, 1116, 1099, 1087, 1070, 1024, 1017, 941, 882, 844, 831, 819, 793, 775, 745, 707, 687, 613, 583, 567, 549, 442.

### Example XIII

### General Ethenolysis Procedure

In a protective atmosphere of argon, (Ru16, Ru-e-i, ca. 5 mg) was placed in a Schlenk flask and dissolved in 5 mL of anh. toluene to prepare the basic solution. Methyl oleate was filtered through an aluminium oxide to a Schlenk flask equipped with a magnetic stirring element and degassed under reduced pressure (p~1×10⁻²). A quantity of the basic solution containing 3 or 1 ppm of the Ru complex was added to methyl oleate under an argon atmosphere. The mixture was immediately transferred to an autoclave containing a glass vessel equipped with a magnetic stirring element. The autoclave was then filled with a 3.5 or 4.5 ethylene (10 bar) and the reaction mixture was stirred at 40° C. for 6 hours. After this time, the pressure was equalised, the autoclave was disassembled, SnatchCat solution in DCM was added and a sample was collected and subjected to GC analysis.

### GC Method Result Analysis Procedure

Before the reactions, samples of methyl oleate and ethenolysis products were used to determine the reaction factors (RF) to GC. Known masses of the substrate and of the products were added to the GC vials, dissolved in toluene and analyses 4 times using GC.

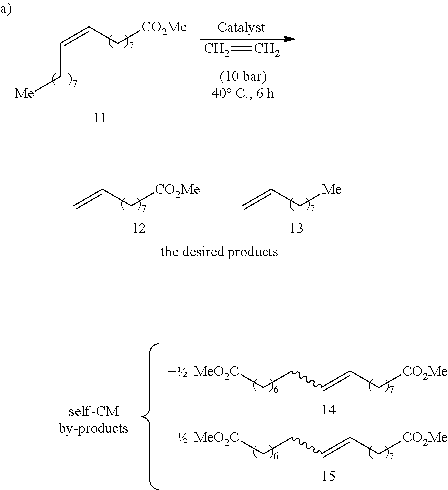

Conversion=100×[1−(A₁₁×A⁰_{IS})/(A⁰₁₁×A_{IS})]; Selectivity=100×(n₁₂+n₁₃)/[(n₁₂+n₁₃)+2×(n₁₄+n₁₅)]; Yield=(conversion×selectivity)/100; TON=Yield×[(n⁰₁₁/n⁰_{[Ru]})]/100; A₁₁, A_{IS}=GC peak area a of methyl oleate and internal standard at the end of the reaction; A⁰₁₁, A⁰_{IS}=GC peak area a of methyl oleate and internal standard before the reaction; n⁰₁₁, n⁰_{[Ru]}=initial number of moles of the used methyl oleate and the catalyst; IS=internal standard (methyl stearate).

TABLE 9
Summary of ethenolysis results

| | | | | Selec- | | |
|---|---|---|---|---|---|---|
| | Loading | Ethylene | Conversion | tivity | Yield | |
| Catalyst | [ppm] | purity | [%] | [%] | [%] | TON |
| Ru-e | 3 | 3.5 | 57 | 95 | 54 | 181,000 |
| | 1 | 4.5 | 38 | 96 | 36 | 362,000 |
| Ru-f | 3 | 3.5 | 36 | 90 | 32 | 107,000 |
| | 1 | 4.5 | 18 | 89 | 16 | 160,000 |
| Ru-i | 3 | 3.5 | 54 | 91 | 49 | 164,000 |
| | 1 | 4.5 | 24 | 92 | 22 | 221,000 |
| Ru-g | 3 | 3.5 | 55 | 97 | 54 | 179,000 |
| | 1 | 4.5 | 27 | 99 | 27 | 270,000 |
| Ru-h | 3 | 3.5 | 68 | 87 | 59 | 197,000 |
| | 1 | 4.5 | 30 | 90 | 27 | 274,000 |

### Example XIV

### RCM Ring Closure Reaction of diethyl 2,2-diallylmalonate (DEDAM)

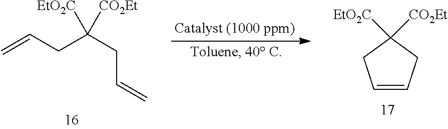 [SMILES: C=CCC(CC=C)(C(=O)OCC)C(=O)OCC ; CCOC(=O)C1(C(=O)OCC)CC=CC1]

### General Procedure for RCM—diethyl 2,2-diallylmalonate

Diethyl 2,2-diallylmalonate (16) was degassed under reduced pressure (p~1×10⁻²) and transferred under a protective argon atmosphere using a glass syringe filter to a Schlenk flask (1 equiv.). The substrate was mixed with the standard (1,3,5-trimethoxybenzene, 1 equiv.). The mixture was degassed and dissolved in anh. toluene (0.1 M). The basic solution of the substrate and of the internal standard was transferred to six Schlenk flasks. In a protective atmosphere of argon, (Ru16, Ru-e-i, ca. 5 mg) was placed in a separate Schlenk flask and dissolved in 2 mL of anh. toluene. The appropriate quantity of the basic catalyst solution (1000 or 100 ppm) was added under argon to the mixture of the substrate and of the internal standard. The solution was stirred at 40° C. for 24 hours. During the reaction, samples for GC analyses (ca. 0.3 mL each) were collected to 1.5 mL vials containing ca. 1 mL of toluene and two drops of the SnatchCat solution in DCM.

TABLE 10
Results of RCM reaction for 1000 ppm of
the catalyst - see FIG. 11a and FIG. 11b

| Time | Catalyst | | | | | |
|---|---|---|---|---|---|---|
| [min] | Ru16 | Ru-e | Ru-f | Ru-i | Ru-g | Ru-h |
| 0 | 0 | 0 | 0 | 0 | 0 | 0 |
| 1 | 13.680 | 5.996 | 37.538 | 31.728 | 0.091 | 0.595 |
| 2 | 42.745 | 17.884 | 73.330 | 73.002 | 0.285 | 4.763 |
| 3 | 77.689 | 37.119 | 91.711 | 94.229 | 1.168 | 11.051 |
| 4 | 93.857 | 61.577 | 97.857 | 98.986 | 1.29 | 19.376 |
| 5 | 98.245 | 82.012 | 99.376 | 99.690 | 1.184 | 29.268 |
| 7 | 99.822 | 96.676 | 99.817 | 99.816 | 1.641 | 50.924 |
| 10 | 100 | 99.596 | 99.851 | 99.866 | 2.517 | 77.344 |
| 15 | 100 | 100 | 99.890 | 99.894 | 3.037 | 95.430 |
| 20 | 100 | 100 | 99.877 | 99.914 | 3.987 | 98.883 |
| 25 | 100 | 100 | 99.896 | 99.922 | 6.364 | 99.734 |
| 30 | 100 | 100 | 99.916 | 99.928 | 8.317 | 99.829 |
| 40 | 100 | 100 | 99.93 | 99.939 | 12.466 | 99.903 |
| 50 | 100 | 100 | 99.94 | 99.949 | 15.927 | 99.909 |
| 60 | 100 | 100 | 99.946 | 99.953 | 23.859 | 99.923 |
| 90 | 100 | 100 | 99.962 | 99.958 | 43.249 | 100 |
| 120 | 100 | 100 | 99.970 | 99.971 | 63.424 | 100 |
| 1440 | 100 | 100 | 100 | 100 | 100 | 100 |

TABLE 11
Results of RCM reaction for 100 ppm of the catalyst-see FIG. 12

| Time | Catalyst | | | | | |
|---|---|---|---|---|---|---|
| [min] | Ru16 | Ru-e | Ru-f | Ru-i | Ru-g | Ru-h |
| 0 | 0 | 0 | 0 | 0 | 0 | 0 |
| 1 | 0.917 | 0.396 | 10.522 | 3.411 | 0 | 0.000 |
| 2 | 3.601 | 1.232 | 19.691 | 8.149 | 0 | 0.000 |
| 3 | 8.077 | 3.539 | 31.921 | 15.491 | 0 | 0.207 |
| 5 | 20.765 | 8.706 | 54.733 | 30.995 | 0 | 1.108 |
| 7 | 35.672 | 14.429 | 66.518 | 43.013 | 0 | 1.721 |
| 10 | 57.909 | 25.716 | 79.658 | 58.776 | 0 | 3.318 |
| 15 | 84.754 | 49.970 | 92.417 | 78.947 | 0 | 5.510 |
| 30 | 100 | 92.857 | 97.557 | 100 | 0 | 16.887 |
| 60 | 100 | 100 | 100 | 100 | 0.633 | 55.984 |
| 120 | 100 | 100 | 100 | 100 | 2.082 | 82.150 |

### Example XV

### RCM Reaction for N-allyl-4-methyl-N-(2-methylallyl)benzenesulphonamide

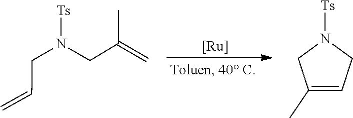

### General RCM Procedure

N-allyl-4-methyl-N-(2-methylallyl)benzenesulphonamide was transferred under protective argon atmosphere to a Schlenk flask (1 equiv.), and subsequently mixed with the internal standard (1,3,5-trimethoxybenzene, 1 equiv.). The mixture was degassed and dissolved in anh. toluene (0.1 M). The basic solution of the substrate and of the internal standard was transferred to twelve 4 mL vials. In a protective atmosphere of argon, (Ru16, Ru-e to Ru-h, ca. 5 mg) was placed in separate Schlenk flasks and dissolved in 2 mL of anh. toluene. The appropriate quantity of the basic catalyst solution (1000 or 100 ppm) was added under argon to the mixture of the substrate and of the internal standard. The solution was stirred at 40° C. for 24 hours. During the reaction, samples for GC analyses (ca. 0.3 mL each) were collected to 1.5 mL vials containing ca. 1 mL of toluene and two drops of the SnatchCat solution in DCM.

TABLE 12
Results of RCM reaction for 1000 ppm of the catalyst-see FIG. 13

| Time | Catalyst | | | | | |
|---|---|---|---|---|---|---|
| [min] | Ru16 | Ru-e | Ru-f | Ru-i | Ru-g | Ru-h |
| 0 | 0 | 0 | 0 | 0 | 0 | 0 |
| 5 | 71.91 | 16.60 | 92.45 | 97.44 | —^{a} | 42.32 |
| 10 | 95.99 | 66.57 | 99.11 | 100 | 6.43 | 81.51 |
| 15 | 98.82 | 89.88 | 99.83 | 100 | 6.38 | 94.25 |
| 30 | 100 | 98.81 | 100 | 100 | 5.96 | 99.56 |
| 60 | 100 | 100 | 100 | 100 | 5.81 | 100 |
| 90 | 100 | 100 | 100 | 100 | 6.16 | 100 |
| 120 | 100 | 100 | 100 | 100 | 5.87 | 100 |
| 1440 | 100 | 100 | 100 | 100 | 100 | 100 |

^{a}The measurement was omitted because of an accidental deviation.

TABLE 13
Results of RCM reaction for 100 ppm of the
catalyst-see FIG. 14a a and FIG. 14b

| | |
|---|---|
| Time | Catalyst |

| [min] | Ru16 | Ru-e | Ru-f | Ru-i | Ru-g | Ru-h |
|---|---|---|---|---|---|---|
| 0 | 0 | 0 | 0 | 0 | 0 | 0 |
| 5 | 10.16 | 2.90 | 49.76 | 31.67 | —^{a} | 7.14 |
| 10 | 12.03 | 2.99 | 79.24 | 69.44 | 0.76 | 8.15 |
| 15 | 15.85 | 5.52 | 89.71 | 85.97 | —^{a} | 9.73 |
| 30 | 41.61 | 19.30 | 96.42 | 96.53 | 3.85 | 14.65 |
| 60 | 84.37 | 78.84 | 98.58 | 99.02 | 4.94 | 30.99 |
| 90 | 93.35 | 92.63 | 98.89 | 99.41 | —^{a} | 39.60 |
| 120 | 95.98 | 96.69 | 99.02 | 99.57 | —^{a} | 47.17 |
| 1440 | 99.32 | 99.69 | 99.01 | 99.64 | 25.19 | 84.92 |

^{a}The measurement was omitted because of an accidental deviation.

### Example XVI

### Self-CM Cross Reaction of Methyl Oleate

Methyl oleate was degassed in vacuo (p~1×10⁻²) i and transferred under a protective argon atmosphere using a glass syringe filter to a Schlenk flask (1 equiv.). The substrate (containing the internal standard—methyl stearate) was transferred to six vials (catalyst loading 1.0 ppm) or a Schlenk flask (catalyst loading 2.5 ppm). In a protective atmosphere of argon, (Ru16, Ru-e-i, ca. 5 mg) was placed in a separate Schlenk flask and dissolved in 5 mL of anh. toluene. The appropriate quantity of the basic catalyst solution was added to the mixture under argon. The solution was stirred at 55° C. for 4 hours (1 ppm) or 24 hours (2.5 ppm). During the reaction, samples for GC analyses (ca. 0.1 mL each) were collected to 1.5 mL vials containing ca. 1 mL of toluene and two drops of the SnatchCat solution in DCM.

TABLE 14
Results for 2.5 ppm of the catalyst-see FIG. 15

| Time | Catalyst | | | | | |
|---|---|---|---|---|---|---|
| [min] | Ru16 | Ru-e | Ru-f | Ru-i | Ru-g | Ru-h |
| 0 | 0 | 0 | 0 | 0 | 0 | 0 |
| 5 | 0 | 0 | 0.075 | 38.757 | 0 | 0 |
| 15 | 0.292 | 0 | 21.196 | 45.731 | 0 | 11.495 |
| 30 | 3.469 | 0 | 41.462 | 47.800 | 0 | 36.123 |
| 60 | 15.015 | 3.882 | 47.282 | 49.270 | 0 | 50.219 |
| 120 | 31.945 | 15.955 | 47.063 | 49.580 | 0 | 49.922 |
| 1440 | 45.765 | 40.063 | 48.954 | 50.453 | 0 | 51.005 |

### Example XVII

### Cross-CM Reaction of Allylbenzene with (Z)-but-2-en-1,4-dioctane

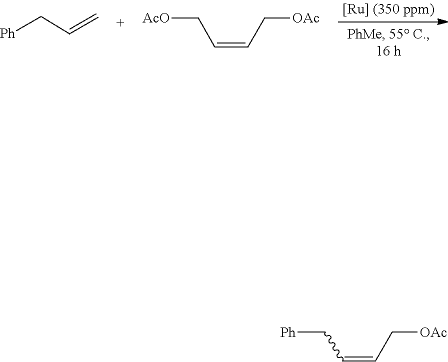

TABLE 15
Reaction results for 1 ppm of the catalyst-see FIG. 16

| | Catalyst | | | | | |
|---|---|---|---|---|---|---|
| Time [min] | Ru16 | Ru-e | Ru-f | Ru-i | Ru-g | Ru-h |
| 0 | 0 | 0 | 0 | 0 | 0 | 0 |
| 5 | 0.136 | 0 | 3.625 | 1.325 | 0 | 0.685 |
| 15 | 0.447 | 0 | 14.608 | 10.516 | 0 | 8.845 |
| 30 | 2.565 | 0.627 | 24.959 | 25.570 | 0 | 24.458 |
| 60 | 4.223 | 3.895 | 32.158 | 38.061 | 0 | 41.407 |
| 120 | 8.440 | 7.464 | 35.165 | 42.782 | 0.686 | 48.114 |
| 180 | 10.525 | 10.906 | 35.575 | 43.977 | 0.843 | 49.454 |
| 240 | 11.933 | 13.364 | 35.570 | 43.662 | 0.151 | 49.445 |

Under a protective argon atmosphere, in a Schlenk flask equipped with a magnetic stirring element, allylbenzene (69 mg, 0.57 mmol) and (Z)-but-2-en-1,4-dioctane (207 mg, 1.14 mmol) were dissolved in anh. PhMe (c=0.1 M). 250 ppm of the Ru catalyst (as basic solution in anhydrous PhMe) was added to this reaction mixture and the reaction mixture was stirred for 4 hours. In the case of Ru-g, additional 100 ppm of Ru catalyst was added after this time and the reaction mixture was additionally stirred for 12 hours. At the end of the reaction, the solvent was evaporated under reduced pressure and the reaction product was purified using quick column chromatography (2% EtOAc in n-hexane).

### Results:

a. Ru16 (92 μg, 0.1 μmol) was used to obtain the expected product (78 mg, 0.41 mmol, 72%);
b. Ru-e (100 μg, 0.1 μmol) was used to obtain the expected product (80 mg, 0.42 mmol, 74%);
c. Ru-f (98 μg, 0.1 μmol) was used to obtain the expected product (80 mg, 0.42 mmol, 74%);
d. Ru-l (100 μg, 0.1 μmol) was used to obtain the expected product (80 mg, 0.42 mmol, 74%);
e. Ru-g (145 μg, 0.2 μmol) was used to obtain the expected product (60 mg, 0.32 mmol, 55%);
f. Ru-h (108 μg, 0.1 μmol) was used to obtain the expected product (82 mg, 0.43 mmol, 75%).

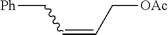 [SMILES: CC(=O)OCC=CCc1ccccc1]

¹H NMR (400 MHz, CDCl₃): δ 7.34-7.28 (m, 2H), 7.25-7.15 (m, 3H), 5.93 (dtt, J=15.3, 6.7, 1.3 Hz, 0.87×1H), 5.84 (dtt, J=10.9, 7.5, 1.3 Hz, 0.13×1H), 5.76-5.58 (m, 1H), 4.78-4.71 (m, 0.13×2H), 4.55 (dq, J=6.4, 1.1 Hz, 0.87×2H), 3.48 (ddd, J=7.5, 1.5, 0.8 Hz, 0.13×2H), 3.44-3.38 (m, 0.87×2H), 2.09 (s, 0.13×3H), 2.07 (s, 0.87×3H); ¹³C NMR (101 MHz, CDCl₃): δ 171.1, 171.0, 139.9, 139.7, 134.7, 133.6, 128.7, 128.7, 128.6, 128.5, 126.4, 126.3, 125.4, 124.4, 65.0, 60.3, 39.1, 38.8, 33.9, 21.2.

### Example XVIII

### Cross-CM Reaction of undck-10-en-1-yl acetate acrylonitrile

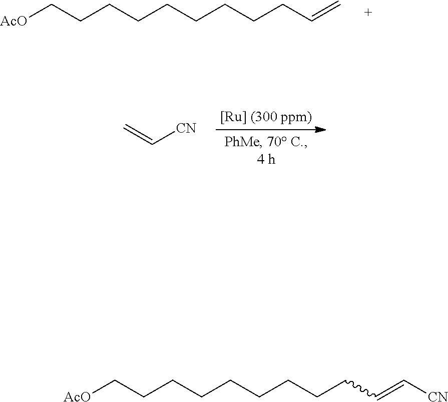

Under a protective argon atmosphere, in a Schlenk flask equipped with a magnetic stirring element, undec-10-en-1-yl acetate (175 mg, 0.82 mmol) and acrylonitrile (88 mg, 1.65 mmol) were dissolved in anh. PhMe (c=0.1 M). 300 ppm of the Ru catalyst (as basic solution in anhydrous PhMe) was added to this reaction mixture and the reaction mixture was stirred for 4 hours. At the end of the reaction, the solvent was evaporated under reduced pressure and the product (25) was purified using quick column chromatography (2%-5% EtOAc in n-hexane).

### Results

a. Ru16 (158 μg, 0.24 μmol) was used to obtain the expected product (100 mg, 0.42 mmol, 51%) as colourless oil.
b. Ru-f (169 μg, 0.24 μmol) was used to obtain the expected product (140 mg, 0.59 mmol, 72%) as colourless oil.
c. Ru-i (172 μg, 0.24 μmol) was used to obtain the expected product (162 mg, 0.68 mmol, 83%) as colourless oil.
d. Ru28 (169 μg, 0.24 μmol) was used to obtain the expected product (120 mg, 0.51 mmol, 61%) as colourless oil.

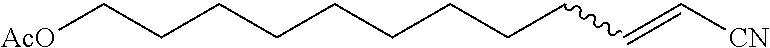 [SMILES: CC(=O)OCCCCCCCCCC=CC#N]

¹H NMR (400 MHz, CDCl3): δ 6.71 (dt, J=16.3, 7.0 Hz, 0.21×1H), 6.47 (dt, J=10.9, 7.7 Hz, 0.79×1H), 5.35-5.28 (m, 1H), 4.05 (t, J=6.7 Hz, 2H), 2.42 (qd, J=7.6, 1.4 Hz, 0.79×2H), 2.25-2.18 (m, 0.21×2H), 2.04 (s, 3H), 1.66-1.57 (m, 2H), 1.52-1.40 (m, 2H), 1.39-1.26 (m, 8H); ¹³C NMR (101 MHz, CDCl3): δ 171.4, 171.4, 156.3, 155.3, 117.7, 116.2, 99.8, 99.7, 77.5, 77.2, 76.8, 64.7, 64.7, 33.4, 32.0, 29.3, 29.3, 29.2, 29.2, 29.0, 29.0, 28.7, 28.3, 27.7, 26.0, 21.2.

1. A precursor of cyclic alkylamine carbenes (CAAC) with the formula CAAC-a, CAAC-b, CAAC-c, CAAC-d, CAAC-e, CAAC-f, CAAC-g, CAAC-h, CAAC-i, CAAC-j, or CAAC-l:

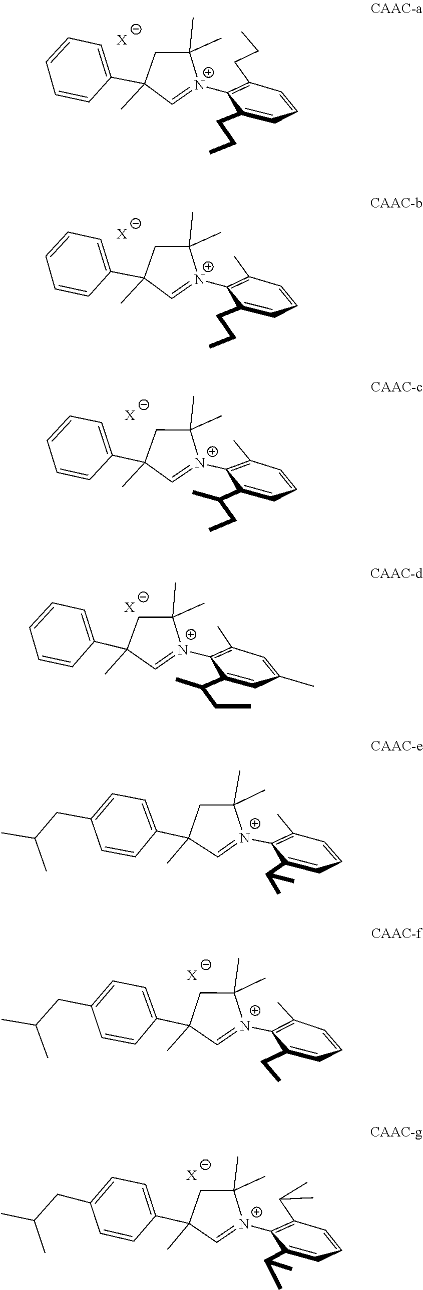

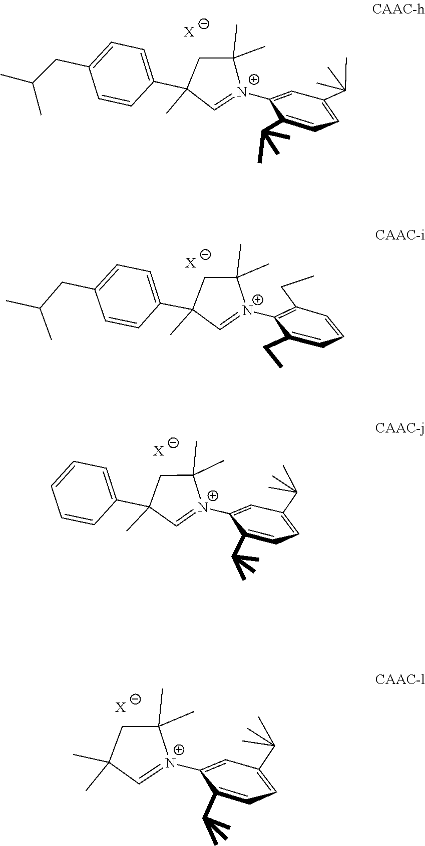

2. Synthesis method of a cyclic precursor of alkylamino carbenes (CAAC) with the formula CAAC-2 or CAAC-3 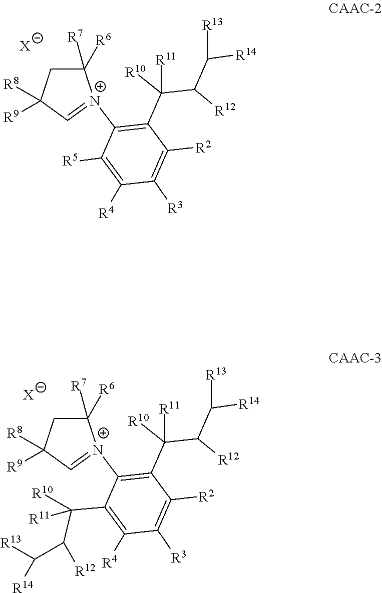
in which
X denotes an anion selected from a group including a halogen anion, BF₄⁻, PF₆⁻, ClO₄⁻, CF₃SO₂O⁻;
R², R³, R⁴ and R⁵ denote independently a hydrogen atom, a C₁-C₁₂ alkyl group, a C₃-C₂ cycloalkyl group, a C₅-C₂₀ aryl or a C₅-C₂₀ heteroaryl group, a C₅-C₂₅ aralkyl group, which may be independently substituted with one and/or more substituents selected from a group including a hydrogen atom, a halogen atom, a C₁-C₂ alkyl group, a C₁-C₂ perfluoroalkyl group, a C₅-C₂₀ aryl group, a C₅-C₂₀ perfluoroaryl group, a C₅-C₂₀ heteroaryl group, a C₁-C₂ alkoxy group, a C₅-C₂₄ aryloxy group, C₅-C₂₀ heteroaryloxy group, a sulphide group (—SR″), an amine group (—NR″₂), in which the R″ group independently denotes a hydrogen atom, a C₁-C₅ alkyl, a C₆-C₂₄ aryl, a C₇-C₂₄ arakyl, alternatively R¹, R², R³, R⁴ and R⁵ are connected forming a C₅-C₂₅ ring
wherein at least one substituent R² and R⁵ has an alkyl substituent with at least one secondary, tertiary or quaternary carbon atom, preferably at least one substituent R¹, R² and R⁵ has an alkyl substituent with at least two secondary, tertiary or quaternary carbon atoms, more preferably at least one substituent R¹, R² and R⁵ has an alkyl substituent with at least three secondary, tertiary or quaternary carbon atoms, most preferably at least one substituent R¹, R² and R⁵ has an alkyl substituent with at least four secondary, tertiary or quaternary carbon atoms,
each substituent R⁶, R⁷, R⁸ and R⁹ denotes a hydrogen atom, a halogen atom, a C₁-C₁₂ alkyl group or a C₅-C₂₀ aryl group which may be independently substituted with one and/or more substituents selected from a group including hydrogen, a C₁-C₁₂ alkyl group, a C₁-C₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently denotes a hydrogen atom, a C₁-C₅ alkyl, a C₆-C₂₄ aryl, a C₇-C₂₄ aralkyl, alternatively R⁶ and R⁷ and/or R⁸ and R⁹ are connected forming a C₅-C₂₅ ring group,
R¹⁰, R¹¹, R¹², R¹³ and R¹⁴ substituents denote independently a hydrogen atom, a C₁-C₁₂ alkyl group, a C₃-C₂ cycloalkyl group, a C₅-C₂₀ aryl group or a C₅-C₂₀ heteroaryl group, a C₅-C₂₅ aralkyl group, which may be independently substituted by one and/or more substituents selected from a group including a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₂ alkoxy group, a C₅-C₂₄ aryloxy group, a C₅-C₂₀ heteroaryloxy group, a sulphide group (—SR″), an amine group (—NR″₂), in which the R″ group independently denotes a hydrogen atom, C₁-C₅ alkyl, C₆-C₂₄ aryl, C₇-C₂₄ aralkyl, alternatively R¹, R², R³, R¹³ and R⁵ are connected and form a C₅-C₂₅ ring.
during the first stage, a compound with the formula 2a or 3a 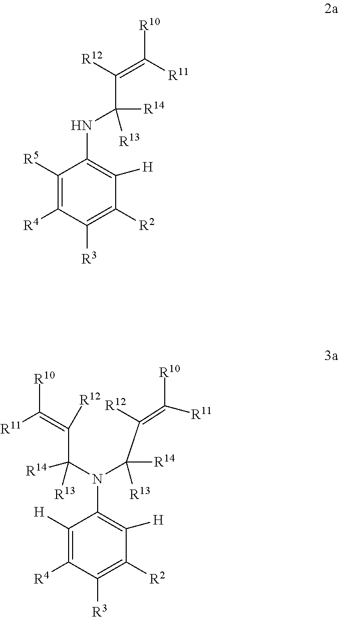
in which the substituents R², R³, R⁴, R⁵, R¹⁰, R¹¹, R¹², R¹³ and R¹⁴ have the meanings defined above are subjected to a thermal rearrangement reaction of the aza-Claisen type in the presence of a Lewis acid, obtaining a compound with the formula 2b or 3b, 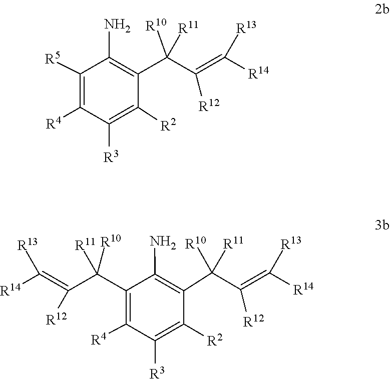
in which the substituents R², R³, R⁴, R¹⁰, R, R¹², R¹³ and R¹⁴ have the meanings defined above,
which is subsequently subjected in the second reaction stage to a reduction of the double bond, using a transition metal catalyst deposited on activated charcoal in the presence of gaseous hydrogen, with the formation of a compound with the formula 2c or 3c 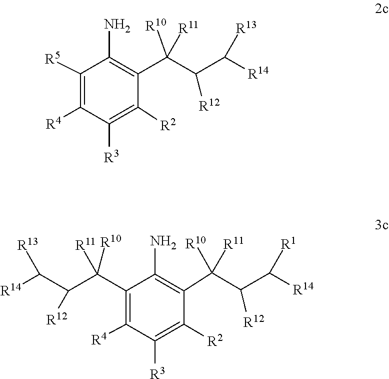
in which the substituents R², R³, R⁴, R¹⁰, R, R¹², R¹³ and R¹⁴ have the meanings defined above,
which is subjected to a reaction with a compound with formula 4 in the third stage 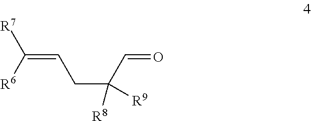
in which R⁶, R⁷, R⁸ and R⁹ have the meanings defined above,
in the presence of a Bronsted acid, forming a compound with the formula 2d or 3d, 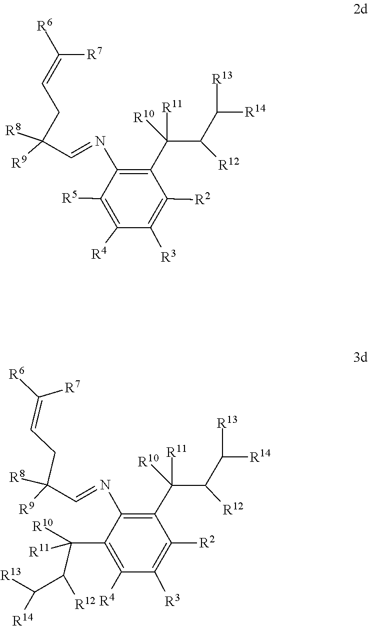
where the R², R³, R⁴, R⁵, R⁶, R⁷, R⁸, R⁹, R¹⁰, R¹¹, R¹², R¹³ and R¹⁴ substituents have the meanings defined above,
which is subsequently subjected to a reaction with hydrochloric acid at an elevated temperature, followed by an anion exchange reaction with an anion selected from a group including a halogen anion, BF₄⁻, PF⁻, ClO₄⁻, CF₃SO₂O⁻, with the formation of the relevant precursors of cyclic alkylamine carbenes (CAAC) with the formula CAAC-2 or CAAC-3.

3. Method according to claim 2, in which
during the first step, a Lewis acid selected from BF₃, B(OR)₃, AlCl₃, MgCl₂, TiCl₄, Ti(OR)₄ is used;
during the second step, the reduction reaction is carried out in the presence of a catalyst selected from Pd, Pt, Rh, Ru, Ag, Au;
in the third step, the reaction is carried out in the presence of a Bronsted acid selected from para-toluenosulphonic acid, sulphuric acid, hydrochloric acid, trifluoroacetic acid, trifluoromethanesulphonic acid, fluorosulphuric acid.

4. A ruthenium complex with the formula 1-Ru 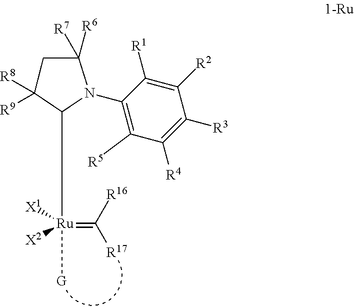
in which:
X¹ and X² independently from each other denote an anionic ligand selected from a group including a halogen anion, a —CN, —SCN, —OR^{a}, —SR^{a}, —O(C═O)R^{a}, —O(SO₂)R^{a} and -oSi(R^{a})₃ group, in which R^{a} denotes a C₁-C₁₂ alkyl, a C₃-C₁₂ cycloalkyl, a C₂-C₂ alkenyl or a C₅-C₂₀ aryl, which is optionally substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom;
R¹, R², R³, R⁴ and R⁵ denote independently a hydrogen atom, a C₁-C₁₂ alkyl group, a C₃-C₂ cycloalkyl group, a C₅-C₂₀ aryl group or a C₅-C₂₀ heteroaryl group, a C₅-C₂₅ aralkyl group, which may be independently substituted by one and/or more substituents selected from a group including a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₂ alkoxy group, a C₅-C₂₄ aryloxy group, a C₅-C₂₀ heteroaryloxy group, a sulphide group (—SR″), an amine group (—NR″₂), in which the R″ group independently denotes a hydrogen atom, C₁-C₅ alkyl, C₆-C₂₄ aryl, C₇-C₂₄ aralkyl, alternatively R¹, R², R³, R⁴ and R⁵ are connected and form a C₅-C₂₅ ring
wherein at least one substituent R¹, R² and R⁵ has an alkyl substituent with at least one secondary, tertiary or quaternary carbon atom, preferably at least one substituent R¹, R² and R⁵ has an alkyl substituent with at least two secondary, tertiary or quaternary carbon atoms, more preferably at least one substituent R¹, R² and R⁵ has an alkyl substituent with at least three secondary, tertiary or quaternary carbon atoms, most preferably at least one substituent R¹, R² and R⁵ has an alkyl substituent with at least four secondary, tertiary or quaternary carbon atoms,
each substituent R⁶, R⁷, R⁸ and R⁹ denotes a hydrogen atom, a halogen atom, a C₁-C₁₂ alkyl group or a C₅-C₂₀ aryl group which may be independently substituted with one and/or more substituents selected from a group including hydrogen, a C₁-C₁₂ alkyl group, a C₁-C₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently denotes a hydrogen atom, a C₁-C₅ alkyl, a C₆-C₂₄ aryl, a C₇-C₂₄ aralkyl, alternatively R⁶ and R⁷ and/or R⁸ and R⁹ are connected forming a C₅-C₂₅ ring group.
R¹⁶ and R¹⁷ independently denote a hydrogen atom, a halogen atom, a C₁-C₂₅ alkyl, possibly substituted, a C₃-C₂₅ cycloalkyl, possibly substituted, a C₁-C₁₂ perfluoroalkyl, possibly substituted, a C₂-C₂₅ alkene, possibly substituted, a C₂-C₂₅ alkenyl, possibly substituted, a C₃-C₂₅ cycloalkenyl, possibly substituted, a C₂-C₂₅ alkynyl, possibly substituted, a C₃-C₂₅ cycloalkynyl, possibly substituted, a C₁-C₂₅ alkoxyl, possibly substituted, a C₅-C₂₅ aryl, possibly substituted, a C₅-C₂₅ aryloxyl, possibly substituted, a C₆-C₂₅ arylalkyl, possibly substituted, a C₅-C₂₅ heteroaryl, possibly substituted, a C₅-C₂₅ heteroaryloxyl, possibly substituted, a C₅-C₂₅ perfluoroaryl, possibly substituted, a 3-12-membered heterocycle containing a sulphur, oxygen, nitrogen, selenium or phosphorus atom, possibly substituted;
wherein the R¹⁶ and R¹⁷ substituents may be connected, forming a ring selected from a group including a C₃-C₂₅ cycloalkyl, a C₃-C₂₅ cycloalkenyl, a C₃-C₂₅ cycloalkynyl, a C₅-C₂₅ aryl, a C₅-C₂₅ heteroaryl, a C₅-C₂₅ perfluoroaryl, a 3-12-membered heterocycle containing a sulphur, oxygen, nitrogen, selenium or phosphorus atom, which may be independently substituted with one and/or more substituents selected from a group including a hydrogen atom, a halogen atom, a C₁-C₂₅ alkyl, a C₃-C₂₅ cycloalkyl, a C₁-C₂ perfluoroalkyl, a C₂-C₂₅ alkene, a C₂-C₂₅ alkenyl, a C₂-C₂₅ cycloalkenyl, a C₂-C₂₅ alkynyl, a C₃-C₂₅ cycloalkynyl, a C₁-C₂₅ alkoxyl, a C₅-C₂₅ aryl, a C₅-C₂₅ aryloxyl, a C₆-C₂₅ arylalkyl, a C₅-C₂₅ heteroaryl, a C₅-C₂₅ heteoaryloxyl, a C₅-C₂₅ perfluoroaryl, a 3-12-membered heterocycle;
where the substituents R¹⁶ and R¹⁷ independently and preferably denote a hydrogen atom and/or a C₅-C₂₅ aryl independently substituted with a hydrogen atom, a halogen atom, a C₁-C₂₅ alkyl group, a C₂-C₂₅ alkenyl group, an alkoxy group (—OR″), a sulphide group (—SR″), a sulphoxide group (—S(O)R″), a sulphonium group (—S⁺R″₂), a sulphone group (—SO₂R″), a sulphonamide group (—SO₂NR″₂), an amine group (—NR″₂), an ammonium group (—N⁺R″₃), a nitro group (—NO₂), a cyanide group (—CN), a phosphonate group (—P(O)(OR″)₂), a phoshphinate group (—P(O)R″(OR″)), a phosphonine group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group independently denotes a hydrogen atom, a C₁-C₅ alkyl, a C₁-C₅ perfluoroalkyl, a C₆-C₂₄ aryl, a C₇-C₂₄ aralkyl, a C₅-C₂₄ perfluoroaryl, two R″ groups may be connected, forming a C₃-C₂ cycloalkyl or a C₃-C₂₅ heterocycloalkyl ring containing a nitrogen, an oxygen or a sulphur atom, possibly additionally substituted with a C₁-C₁₂ alkyl group, alternatively R″ denote a ketone group (—COR^{c}), in which R^{c} denotes a C₁-C₂ perfluoroalkyl or an alkoxy group (—OR^{d}), in which R^{d} denote a C₁-C₁₂ alkyl or a C₃-C₁₂ heterocycloalkyl containing a nitrogen atom, an oxygen atom or a sulphur atom, possibly additionally substituted with a C₁-C₁₂ alkyl group;
G is selected from entities such as
ligand with the formula CAAC-5 or CAAC-6 or CAAC-7 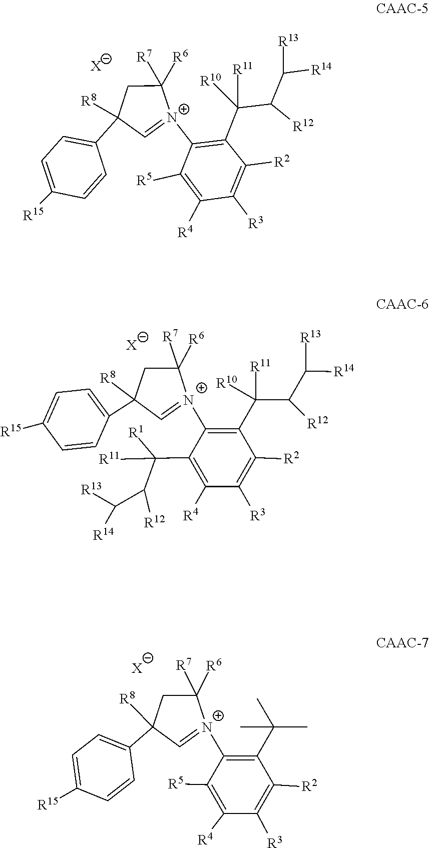
in which X and the substituents R¹ to R¹⁵ have the meanings defined above
R15 independently denotes, a C1-C12 alkyl group, a C3-C12 cycloalkyl group, a C5-C20 aryl or a C5-C20 heteroaryl group, a C5-C25 aralkyl group, which may be independently substituted by one and/or more substituent selected from a group including a hydrogen atom, a halogen atom, a C1-C12 alkyl group, a C1-C12 perfluoroalkyl group, a C5-C20 aryl group, a C5-C20 perfluoroaryl group, a C5-C20 heteroaryl group, a C1-C12 alkoxy group, a C5-C24 aryloxy group, a C5-C20 heteroaryloxy group, an alkoxy group (—OR″), a sulphide group (—SR″), an amine group (—NR″2), in which the R″ group independently denotes a hydrogen atom, a C1-C5 alkyl, a C6-C24 aryl, a C7-C24 aralkyl,
or
heteroatom 1
selected from a group including an oxygen, sulphur, selenium atom, substituted with a group selected from such as a hydrogen atom, a halogen atom, an oxygen atom, a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₂ alkenyl, a C₆-C₂₀ heteroaryl or a C₅-C₂₄ heteroaryloxyl, a 3-12-membered heterocycle, optionally substituted with an acyl (—COR′), a cyano (—CN), a carboxy (—COOH), an ester (—COOR′), an ester (—CH₂COOR′), an ester (—CHR′COOR′), an ester (—C(R′)₂COOR′), an amide (—CONR′₂), a Weinreb type amide (—CON(R′)(OR′)), a sulphone (—SO₂R′), a formyl (—COH), a sulphonamide (—SO₂NR′₂), a ketone (—COR′), a thioamide (—CSNR′₂), a thioketone (—CSR′), a thionoester (—CSOR′), a thioester (—COSR′), a dithioester (—CS₂R′) group, in which the R′ group denotes independently a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl, a C₅-C₂₄ heteroaryloxyl and then the dashed line denotes a direct bond between the heteroatom and the R¹⁷ substituent or a bond between the R¹⁷ substituent and the heteroatom via methylene bridge —CH₂—, —CHR′— or —CR′₂—, wherein the R¹⁴ substituent is a C₅-C₁₅ aryl, possibly substituted with 1-4 substituents independently selected from a group including a hydrogen atom, a halogen atom, a C₁-C₂₅ alkyl, a C₃-C₂₅ cycloalkyl, a C₂-C₂₅ alkenyl, a C₃-C₂₅ cycloalkenyl, a C₂-C₂₅ alkynyl, a C₃-C₂₅ cycloalkynyl, a C₁-C₂₅ perfluoroalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₆-C₂₀ heteroaryl or C₅-C₂₄ heteroaryloxyl, 3-12-membered heterocycle, an alkoxy group (—O″″), a sulphide group (—S″″), a sulphoxide group (—S(O)″″), a sulphonium group (—S⁺″″₂), a sulphone group (—SO₂″″), a sulphonamide group (—SO₂N″″₂), an amine group (—N″″₂), an ammonium group (—N⁺″″3), a nitro group (—NO₂), a cyanide group (—CN), a phosphonate group (—P(O)(O″″)₂), a phosphinate group (—P(O)″″(O″″)), a phosphonine group (—P(O″″)₂), a phosphine group (—P″″₂), a phosphine oxide group (—P(O)″″₂), a phosphonium group (—P⁺″″₃), a carboxy group (—COOH), an ester group (—COO″″), an amide group (—CON″″₂), an amide group (—N″″C(O)″″), a formyl group (—CHO), a ketone group (—CO″″), a thioamide group (—CSN″″₂), a thioketone group (—CS″″), a thionoester group (—CSO″″), a thioester group (—COS″″), a dithioester group (—CS₂″″), in which the ″″ group denotes a C₁-C₅ alkyl, a C₁-C₅ perfluoroalkyl, a C₆-C₂₄ aryl, a C₇-C₂₄ aralkyl, a C₅-C₂₄ perfluoroaryl; or
heteroatom 2
selected from a group including a nitrogen atom or a phosphorus atom, substituted with a group selected from groups such as hydrogen atom, methylidene, possibly substituted with a R′ substituent, a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₂ alkenyl, a C₆-C₂₀ alkenyl or a C₅-C₂₄ heteroaryloxyl, 3-12-membered heterocycle, an acyl group (—COR′), an ester group (—COOR′), 1 tert-butylckarboxycarbon group (t-Boc) or a 9-fluorenylmethoxycarbonyl group (Fmoc), a carbamine group (—CONR′₂), a sulphone group (—SO₂R′), a formyl group (—COH), in which the R′ group denotes a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₂ alkenyl, a C₆-C₂₀ heteroaryl or a C₅-C₂₄ heteroaryloxyl, optionally substituted with an acyl group (—COR′), a cyano group (—CN), a carboxy group (—COOH), an ester group (—COOR′), an ester group (—CH₂COOR′), an ester group (—CHR′COOR′), an ester group (—C(R′)₂COOR′), an amide group (—CONR′₂), a sulphone group (—SO₂R′), a formyl group (—COH), a sulphonamide group (—SO₂NR′₂), a keton group (—COR′), a thioamide group (—CSNR′₂), a thioketone group (—CSR′), a thionoester group (—CSOR′), a thioester group (—COSR′), a dithioester group (—CS₂R′), in which the R′ group denotes a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl or a C₅-C₂₄ heteroaryloxyl, and then the dashed line denotes a direct bond between a heteroatom and the R¹⁴ substituent or denotes a bond between the R¹⁷ substituent and the heteroatom via a methylene bridge (CH₂)—, —(CHR′)— or (CR′₂)—; wherein the R¹⁷ is an C₅-C₁₅ aryl, possibly substituted with 1-4 substituents independently selected from a group including a hydrogen atom, a halogen atom, a C₁-C₂₅ alkyl, a C₃-C₂₅ cycloalkyl, a C₂-C₂₅ alkenyl, a C₃-C₂₅ cycloalkenyl, a C₂-C₂₅ alkynyl, a C₃-C₂₅ cycloalkynyl, a C₁-C₂₅ perfluoroalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₆-C₂₀ heteroaryl or a C₅-C₂₄ heteroaryloxyl, 3-12-membered heterocycle, an alkoxy group (—OR″), a sulphide group (—SR″), a sulphoxide group (—S(O)R″), a sulphonium group (—S⁺R″₂), a sulphone group (—SO₂R″), a sulphonamide group (—SO₂NR″₂), an amine group (—NR″₂), an ammonium group (—N⁺R″₃), a nitro group (—NO₂), a cyano group (—CN), a phosphinous group (—P(O)(OR″)₂), a phosphinic group (—P(O)R″(OR″)), a phosphonine group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes a C₁-C₅ alkyl, a C₁-C₅ perfluoroalkyl, a C₆-C₂₄ aryl, a C₇-C₂₄ aralkyl, a C₅-C₂₄ perfluoroaryl, or
heteroatom 3
selected from a group including a halogen atom and then the dashed line denotes a direct bond between the heteroatom and the R¹⁷ substituent, wherein the R¹⁷ substituent is a C₅-C₁₅ aryl, or a C₅-C₂₅ polyaryl, possibly substituted with 1-4 substituents selected independently from a group including a hydrogen atom, a halogen atom, a C₁-C₂₅ alkyl, a C₃-C₂₅ cycloalkyl, a C₂-C₂₅ alkenyl, a C₃-C₂₅ cycloalkenyl, a C₂-C₂₅ alkynyl, a C₃-C₂₅ cycloalkynyl, a C₁-C₂₅ perfluoroalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₆-C₂₀ heteroaryl or a C₅-C₂₄ heteroaryloxyl, 3-12-membered heterocycle, an alkoxy group (—OR″), a sulphide group (—SR″), a sulphoxide group (—S(O)R″), a sulphonium group (—S⁺R″₂), a sulphone group (—SO₂R″), a sulphonamide group (—SO₂NR″₂), an amine group (—NR″₂), an ammonium group (—N⁺R″₃), a nitro group (—NO₂), a cyano group (—CN), a phosphonous group (—P(O)(OR″)₂), a phosphinous group (—P(O)R″(OR″)), a phosphonin group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R″), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes a C₁-C₅ alkyl, a C₁-C₅ perfluoroalkyl, a C₆-C₂₄ aryl, a C₇-C₂₄ aralkyl, a C₅-C₂₄ perfluoroaryl.

5. A ruthenium complex according to claim 4 represented with the formula 1a-Ru 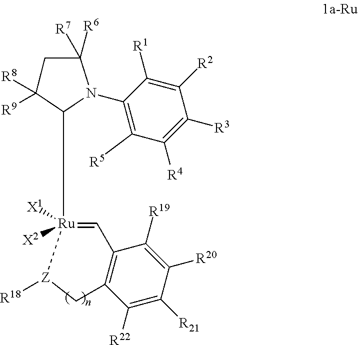
in which X¹ and X² and the R¹, R², R³, R⁴, R⁵, R⁶, R⁷, R⁸, R⁹ substituents have the meanings defined above
‘n’ means 1 or 0
Z is selected from a group including halogen atoms, 0 atom, S atom, Se atom, or a NR′″ group, in which R′″ denotes methylidene, a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₂ alkenyl, a C₆-C₂₀ heteroaryl or a C₅-C₂₄ heteroaryloxyl, 3-12-membered heterocycle, an acyl group (—COR′), an ester group (—COOR′), a tert-butylcarboxycarbon group (t-Boc) or a 9-fluorenylmethoxycarbonyl group (Fmoc), a carbamine group (—CONR′₂), a sulphone group (—SO₂R′), a formyl group (—COH), in which the R′ group denotes a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl or a C₅-C₂₄ heteroaryloxyl, or a halogen atom, wherein if Z denotes a halogen atom, R¹⁸ is absent;
R¹⁸ denotes independently a hydrogen atom, a C₁-C₂₅ alkyl, a C₁-C₂₅ cycloalkyl, a C₅-C₂₀ alkoxyl, a C₅-C₂₀ aryl, a C₅-C₂₄ aryloxyl, a —COOR′″ group, a —CH₂COOR′″ group, a —CONR′″₂ group, a —CH₂CONR′″₂ group, a —COR′″ group, a —CH₂COR′″ group, a —CON(OR′″)(R′″) group, a —CH₂CON(OR′″)(R′″) group or a halogen atom, wherein ″″ denotes a C₁-C₁₂ alkyl, a C₃-C₂ cycloalkyl, a C₂-C₁₂ alkenyl, a C₆-C₂₀ aryl which are possibly substituted with at least one C₁-C₂ alkyl, a C₁-C₁₂ perfluoroalkyl, a C₁-C₁₂ alkoxyl, a C₆-C₂₄ aryloxyl or a halogen atom;
R¹⁹, R²⁰, R²¹ and R²² denote independently a hydrogen atom, a halogen atom, a C₁-C₂₅ alkyl group, a C₂-C₂₅ alkenyl group, a C₅-C₂₅ aryl group, a (—Or″) alkoxy group, a sulphide group (—SR″), a sulphoxide group (—S(O)R″), a sulphonium group (—S⁺R″₂), a sulphone group (—SO₂R″), a sulphonamide group (—SO₂NR″₂), an amine group (—NR″₂), an ammonium group (—N⁺R″₃), a nitro group (—NO₂), a cyano group (—CN), a phosphoric group (—P(O)(OR″)₂), a phosphorous group (—P(O)R″(OR″)), a phosphonine group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R′), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes a C₁-C₅ alkyl, a C₁-C₅ perfluoroalkyl, a C₆-C₂₄ aryl, a C₇-C₂₄ aralkyl, a C₅-C₂₄ perfluoroaryl, wherein the substituents R¹⁶, R¹⁷, R¹⁸ and R¹⁹ may be connected, forming a substituted or an unsubstituted cyclic system C₄-C₁₀ or a polycyclic system C₄-C₁₂.

6. A ruthenium complex according to claim 4 or 5 with the formula 1b-Ru 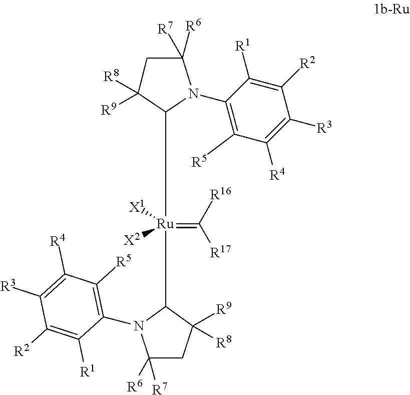
in which X¹ and X² and the R¹, R², R³, R⁴, R⁵, R⁶, R⁷, R⁸, R⁹ substituents have the meanings defined above
R¹⁶ and R¹⁷ independently denote a hydrogen atom, a halogen atom, a C₁-C₂₅ alkyl, possibly substituted, a C₃-C₂₅ cycloalkyl, possibly substituted, a C₁-C₁₂ perfluoroalkyl, possibly substituted, a C₂-C₂₅ alkene, possibly substituted, a C₂-C₂₅ alkenyl, possibly substituted, a C₃-C₂₅ cycloalkenyl, possibly substituted, a C₂-C₂₅ alkynyl, possibly substituted, a C₃-C₂₅ cycloalkynyl, possibly substituted, a C₁-C₂₅ alkoxyl, possibly substituted, a C₅-C₂₅ aryl, possibly substituted, a C₅-C₂₅ aryloxyl, possibly substituted, a C₆-C₂₅ arylalkyl, possibly substituted, a C₅-C₂₅ heteroaryl, possibly substituted, a C₅-C₂₅ heteroaryloxyl, possibly substituted, a C₅-C₂₅ perfluoroaryl, possibly substituted, a 3-12-membered heterocycle containing a sulphur, oxygen, nitrogen, selenium or phosphorus atom, possibly substituted;
wherein the R¹⁶ and R¹⁷ substituents may be connected, forming a ring selected from a group including a C₃-C₂₅ cycloalkyl, a C₃-C₂₅ cycloalkenyl, a C₃-C₂₅ cycloalkynyl, a C₅-C₂₅ aryl, a C₅-C₂₅ heteroaryl, a C₅-C₂₅ perfluoroaryl, a 3-12-membered heterocycle containing a sulphur, oxygen, nitrogen, selenium or phosphorus atom, which may be independently substituted with one and/or more substituents selected from a group including a hydrogen atom, a halogen atom, a C1-C₂₅ alkyl, a C₃-C₂₅ cycloalkyl, a C₁-C₂ perfluoroalkyl, a C₂-C₂₅ alkene, a C₂-C₂₅ alkenyl, a C₃-C₂₅ cycloalkenyl, a C₂-C₂₅ alkynyl, a C₃-C₂₅ cycloalkynyl, a C₁-C₂₅ alkoxyl, a C₅-C₂₅ aryl, a C₅-C₂₅ aryloxyl, a C₆-C₂₅ arylalkyl, a C₅-C₂₅ heteroaryl, a C₅-C₂₅ heteoaryloxyl, a C₅-C₂₅ perfluoroaryl, a 3-12-membered heterocycle.

7. A ruthenium complex according to any of the claims 4-6 with the formula 1c-Ru or 1d-Ru or 1e-Ru 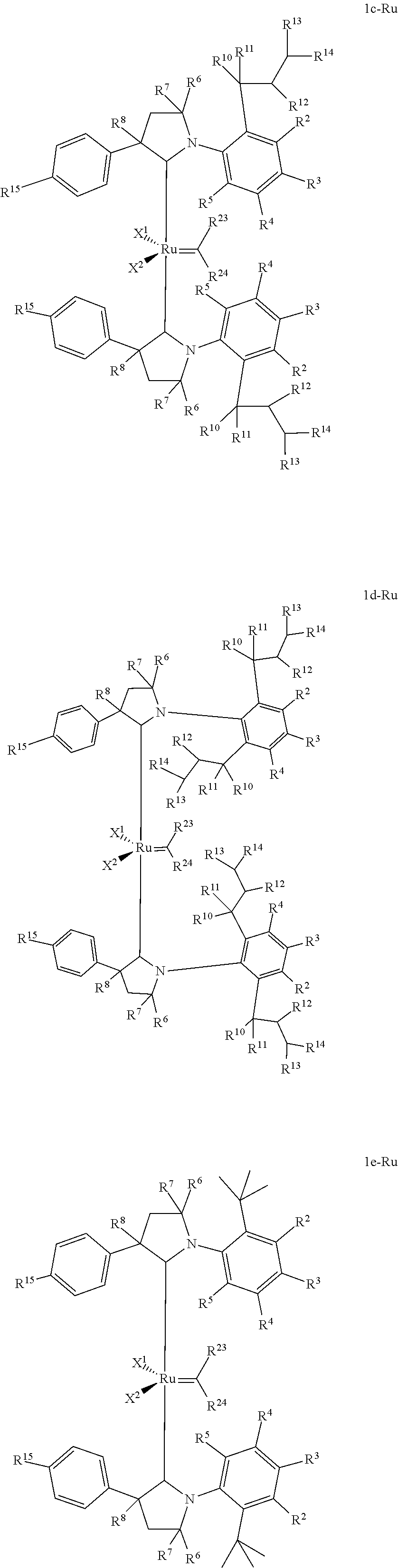
in which X¹ and X² as well as the R¹, R², R³, R⁴, R⁵, R⁶, R⁷, R⁸, R¹⁰, R¹¹, R¹², R¹³, R¹⁴, R¹⁵, R²³, R²⁴ substituents have the meanings defined above.

8. A ruthenium complex according to any of the claims 4-7 selected from the complexes represented with the formulae Ru-e, Ru-f, Ru-g, Ru-h, Ru-i, Ru-l:

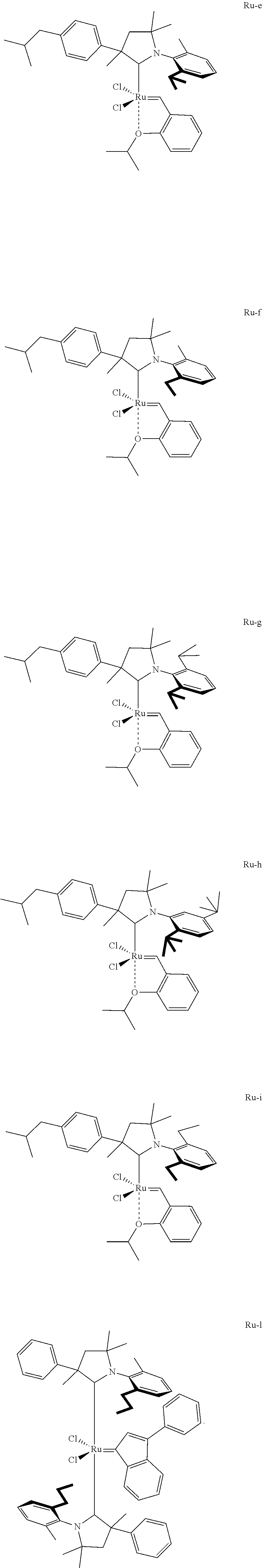

9. Application of the compound with the formula 1-Ru, defined in any of the claims 4-8 as a pre-catalyst and/or a catalyst in olefin metathesis reactions, in particular in ring closure metathesis reactions (RCM), cross-metathesis (CM), homometathesis (cross metathesis between two molecules of the same olefin), ethenolysis, isomerisation, in a methathetic reaction of diastereoselective ring rearrangement (DRRM), “alken-alkin” (en-yn) type metathesis or polymerisation reaction of the ROMP or ADMET type.

10. An application according to claim 9, in which the reaction is carried out in an organic solvent, such as toluene, mesitylene, hexane, cyclohexane, ethyl acetate, methyl acetate, methyl carbonate, ethyl carbonate, tert-butyl-methyl ether, cyclopentyl-methyl ether, diethyl ether, THF, 2-Me-THF, 4-Me-THP, dioxane, DME, PAO. PEG, paraffin, esters of saturated fatty acids.

11. An application according to claim 9, in which the reaction is carried out in a solvent-free system.

12. An application according to any of the above claims, in which the reaction is carried out at 20 to 200° C.

13. An application according to any of the above claims, in which the reaction is carried out for 5 minutes to 48 hours.

14. An application according to any of the above claims, in which the 1-Ru compound is used in a quantity not greater than 10% mol.

15. An application according to any of the above claims, in which the 1-Ru compound is used in a quantity not greater than 0.1% mol.

16. An application according to any of the above claims, in which the 1-Ru compound is added to the reaction mixture in portions, as a solid and/or continuously, using a pump, as a solution in an organic solvent.

17. An application according to any of the above claims, in which the gaseous byproduct of the reaction, selected from ethylene, propylene, butylene, is actively removed from the reaction mixture using inert gas barbotage or under reduced pressure.

18. A ruthenium complex with the formula 1aa-Ru 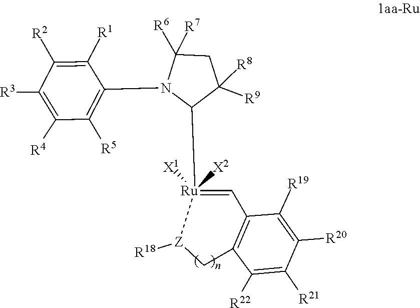
in which X¹ and X² independently from each other denote an anionic ligand selected from a group including a halogen anion, a —CN, —SCN, —OR^{a}, —SR^{a}, —O(C═O)R^{a}, —O(SO₂)R^{a} and —OSi(R^{a})₃ group, in which R^{a} denotes a C₁-C₁₂ alkyl, a C₃-C₁₂ cycloalkyl, a C₂-C₂ alkenyl or a C₅-C₂₀ aryl, which is optionally substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom;
R¹, R², R³, R⁴ and R⁵ denote independently a hydrogen atom, a C₁-C₁₂ alkyl group, a C₃-C₂ cycloalkyl group, a C₅-C₂₀ aryl group or a C₅-C₂₀ heteroaryl group, a C₅-C₂₅ aralkyl group, which may be independently substituted by one and/or more substituents selected from a group including a hydrogen atom, a halogen atom, a C₁-C₁₂ alkyl group, a C₁-C₁₂ perfluoroalkyl group, a C₅-C₂₀ aryl group, a C₅-C₂₀ perfluoroaryl group, a C₅-C₂₀ heteroaryl group, a C₁-C₂ alkoxy group, a C₅-C₂₄ aryloxy group, a C₅-C₂₀ heteroaryloxy group, a sulphide group (—SR″), an amine group (—NR″₂), in which the R″ group independently denotes a hydrogen atom, C₁-C₅ alkyl, C₆-C₂₄ aryl, C₇-C₂₄ aralkyl, alternatively R¹, R², R³, R⁴ and R⁵ are connected and form a C₅-C₂₅ ring
wherein at least one substituent R¹, R², R³, R⁴ and R⁵ has an alkyl substituent with at least one quaternary carbon atom, preferably at least one substituent R¹, R⁴ and R⁵ has an alkyl substituent with at least two quaternary carbon atoms, more preferably at least one substituent R¹, R⁴ and R⁵ has an alkyl substituent with at least three quaternary carbon atoms,
each substituent R⁶ and R⁷ denotes a hydrogen atom, a halogen atom, a C₁-C₁₂ alkyl group or a C₅-C₂₀ aryl group, which may be substituted independently by one and/or more substituents selected from a group including hydrogen, a C₁-C₁₂ alkyl group, a C₁-C₂ perfluoroalkyl group, a C₅-C₂₀ aryl group, a C₅-C₂₀ perfluoroaryl group, a C₅-C₂₀ heteroaryl group, a C₁-C₂ alkoxy group, a C₅-C₂₄ aryloxy group, a C₅-C₂₀ heteroaryloxy group or a halogen atom, a sulphide group (—SR″), an amine group (—NR″₂), in which the R″ group denotes independently a hydrogen atom, a C₁-C₅ alkyl, a C₆-C₂₄ aryl, a C₇-C₂₄ aralkyl, alternatively R⁶ and R⁷ and/or R⁸ and R⁹ are connected forming a C₅-C₂₅ ring group;
the R⁸ substituent denotes a C₅-C₂₀ aryl group, which is independently substituted with one and/or more substituent selected from a group including hydrogen, a C₁-C₁₂ alkyl group, a C₁-C₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently denotes a hydrogen atom, a C₁-C₅ alkyl, a C₆-C₂₄ aryl, a C₇-C₂₄ aralkyl,
the R⁹ substituent denotes a hydrogen atom, a halogen atom, a C₁-C₁₂ alkyl group, which may be independently substituted with one and/or more substituent selected from a group including hydrogen, a C₁-C₁₂ alkyl group, a C₁-C₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently denotes a hydrogen atom, a C₁-C₅ alkyl, a C₆-C₂₄ aryl, a C₇-C₂₄ aralkyl,
R⁸ and R⁹ are alternatively connected and form a C₅-C₂₅ ring group.
‘n’ means 1 or 0;
Z is selected from a group including halogen atoms, 0 atom, S atom, Se atom, or a NR′″ group, in which R′″ denotes methylidene, a C₁-C₂₅ alkyl, a C₁-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₂ alkenyl, a C₆-C₂₀ heteroaryl or a C₅-C₂₄ heteroaryloxyl, 3-12-membered heterocycle, an acyl group (—COR′), an ester group (—COOR′), a tert-butylcarboxycarbon group (t-Boc) or a 9-fluorenylmethoxycarbonyl group (Fmoc), a carbamine group (—CONR′₂), a sulphone group (—SO₂R′), a formyl group (—COH), in which the R′ group denotes a C₁-C₂₅ alkyl, a C1-C₂₅ perfluoroalkyl, a C₃-C₂₅ cycloalkyl, a C₅-C₂₀ alkoxyl, a C₅-C₂₀ aryl, a C₅-C₂₀ perfluoroaryl, a C₇-C₂₀ aralkyl, a C₅-C₂₄ aryloxyl, a C₂-C₁₂ alkenyl, a C₆-C₂₀ heteroaryl or a C₅-C₂₄ heteroaryloxyl,
or a halogen atom, wherein if Z denotes a halogen atom, R¹⁸ is absent;
R¹⁸ means independently a halogen atom, a C₁-C₂₅ alkyl, a C₁-C₂₅ cycloalkyl, a C₅-C₂₀ alkoxyl, a C₅-C₂₀ aryl, a C₅-C₂₄ aryloxyl, a —COOR′″ group, a —CH₂COOR′″ group, a —CONR′″₂ group, a —CH₂CONR′″₂ group, a —COR′″ group, a —CH₂COR′″ group, a —CON(OR′″)(R′″) group, a —CH₂CON(OR′″)(R′″) group or a halogen atom, wherein R′″ denotes a C₁-C₁₂ alkyl, a C₃-C₂ cycloalkyl, a C₂-C₁₂ alkenyl, a C₆-C₂₀ aryl, which are possibly substituted with at least C₁-C₁₂ alkyl, a C₁-C₂perfluoroalkyl, a C₁-C₁₂ alkoxyl, a C₆-C₂₄ aryloxyl or a halogen atom;
R¹⁹, R²⁰, R²¹ and R²² denote independently a hydrogen atom, a halogen atom, a C₁-C₂₅ alkyl group, a C₂-C₂₅ alkenyl group, a C₅-C₂₅ aryl group, an alkoxy group (—OR″), a sulphide group (—SR″), a sulphoxide (—S(O)R″), a sulphonium group (—S⁺R″₂), a sulphone group (—SO₂R″), a sulphonamidowe group (—SO₂NR″₂), an amine group (—NR″₂), an ammonium group (—N⁺R″₃), a nitro group (—NO₂), a cyano group (—CN), a phosphonous group (—P(O)(OR″)₂), a phosphinous group (—P(O)R″(OR″)), a phosphonine group (—P(OR″)₂), a phosphine group (—PR″₂), a phosphine oxide group (—P(O)R″₂), a phosphonium group (—P⁺R″₃), a carboxy group (—COOH), an ester group (—COOR″), an amide group (—CONR″₂), an amide group (—NR″C(O)R′), a formyl group (—CHO), a ketone group (—COR″), a thioamide group (—CSNR″₂), a thioketone group (—CSR″), a thionoester group (—CSOR″), a thioester group (—COSR″), a dithioester group (—CS₂R″), in which the R″ group denotes a C₁-C₅ alkyl, a C₁-C₅ perfluoroalkyl, a C₆-C₂₄ aryl, a C₇-C₂₄ aralkyl, a C₅-C₂₄ perfluoroaryl, wherein the R¹⁶, R¹⁷, R¹⁸ and R¹⁹ substituents may be connected, thus forming a substituted or an unsubstituted C₄-C₁₀ cyclic or a C₄-C₁₂ polycyclic system.

19. A ruthenium complex according to claim 18 represented with the formula 1aaa-Ru 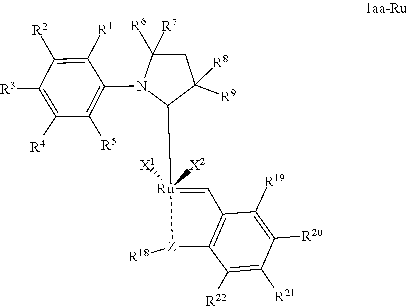
in which X¹ and X² and the R¹, R², R³, R⁴, R⁵, R⁶, R⁷, R⁸, R⁹ substituents have the meanings defined above in claim 21;
Z is selected from a group including halogen atoms, 0 atom, S atom, Se atom.

20. A ruthenium complex according to claim 18 or 19 with the formula 1aaaa-Ru 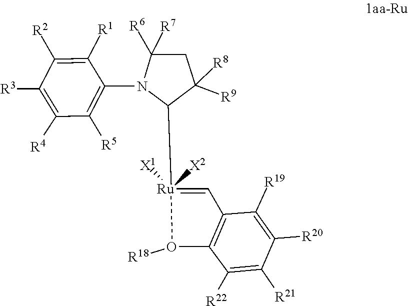
in which X¹ and X² and the R¹, R², R³, R⁴, R⁵, R⁶, R⁷, R⁸ substituents have the meanings defined above in claim 21;
Ar denotes a C₆-C₂₀ aryl group, which may be independently substituted by one and/or more substituents selected from a group including hydrogen, a C₁-C₁₂ alkyl group, a C₁-C₂ perfluoroalkyl group, a C₅-C₂₀ aryl group, a C₅-C₂₀ perfluoroaryl group, a C₅-C₂₀ heteroaryl group, a C₁-C₁₂ alkoxy group, a C₅-C₂₄ aryloxy group, a C₅-C₂₀ heteroaryloxy group or a halogen atom, a sulphide group (—SR″), an amine group (—NR″₂), in which the R″ group independently denotes a hydrogen atom, a C₁-C₅ alkyl, a C₆-C₂₄ aryl, a C₇-C₂₄ aralkyl.

21. A ruthenium complex according to claims 18 to 20, in which at least one substituent among R¹, R², R³, R⁴, R⁵ denotes a tert-butyl or a tert-amyl substituent.

22. Synthesis method of a ruthenium complex with the formula 1a-Ru 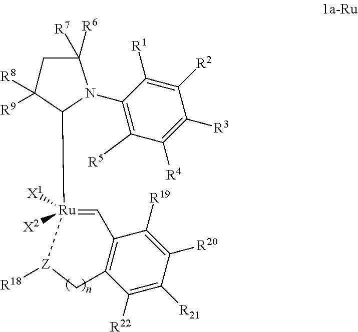
defined in claim 7
characterised in that the alkylidene ruthenium complex with the formula 10 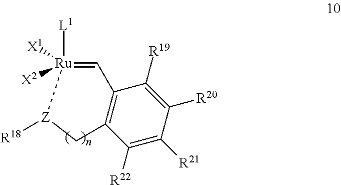
in which:
L¹ denote a neutral ligand selected from a group including pyridine or a substituted pyridine, P(″)₃, P(O″)₃, O(″)₂, N(″)₃, in which each ″ independently denotes a C₁-C₁₂ alkyl, a C₃-C₁₂ cycloalkyl, a C₅-C₂₀ aryl, a C₇-C₂₄ aralkyl, a C₅-C₂₄ perfluoroaryl, 5-12-membered heteroaryl;
N, Z, X¹, X² and the substituents R¹⁸, R¹⁹, R²⁰, R²¹ and R²² have the meanings defined above are subjected to a reaction with a carbene with the formula 8 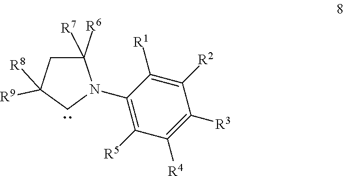
in which the substituents R¹ to R⁹ have the meanings defined above.
